(19) United States
(12) Patent Application Publication
(10) Pub. No.: US 2005/0197342 A1
(43) Pub. Date: Sep. 8, 2005

(54) Substituted nitrogen-containing six-membered amino-heterocycles as vanilloid-1 receptor antagonists for treating pain

(76) Inventors: Gregory John Hollingworth, Brentwood (GB); A. Brian Jones, Saffron Walden (GB); Edward Giles McIver, Sawbridgeworth (GB); Christopher Richard Moyes, Hertford (GB); Lauren Rogers, Braintree (GB)

Correspondence Address: MERCK AND CO., INC, P O BOX 2000, RAHWAY, NJ, 07065-0907, US

(21) Appl. No.: 10/984,336

(22) Filed: Nov. 9, 2004

Related U.S. Application Data
(60) Provisional application No. 60/617,134, filed on Oct. 8, 2004.

(30) Foreign Application Priority Data
Nov. 10, 2003 (GB) 0326217.7
Apr. 5, 2004 (GB) 0407748.3

(51) Int. Cl. A61K 31/506; A61K 31/501; A61K 31/497; C07D 43/02

(52) U.S. Cl. 514/252.04; 514/255.05; 514/275; 544/238; 544/330; 544/405

(57) ABSTRACT

The present invention provides a compound of formula I:
Y-J-NH-Z (I) wherein:
Y is a quinoline or isoquinoline optionally substituted with one or two substituents independently chosen from hydroxy, halogen, haloC₁₋₄alkyl, C₁₋₄alkyl, C₁₋₄alkoxy, haloC₁₋₄alkoxy, nitro and amino;
J is pyridine, pyridazine, pyrazine, pyrimidine or triazine optionally substituted with one or two substituents independently chosen from hydroxy, halogen, haloC₁₋₄alkyl, C₁₋₄alkyl, C₃₋₅cycloalkyl, C₁₋₄alkoxy, hydroxyC₁₋₄alkyl, cyano, hydroxy, C₁₋₄cycloalkoxy, C₁₋₄alkylthio, haloC₁₋₄alkoxy, nitro, Q, (CH₂)ₚQ, —NR²R³, —(CH₂)ₚNR²R³ and —O(CH₂)ₚNR²R³; wherein J is substituted at positions meta to each other by NH and Y; and
Z is phenyl or pyridyl optionally substituted with one or two substituents independently selected from halogen, haloC₁₋₄alkyl, C₁₋₄alkyl, C₁₋₄alkoxy, haloC₁₋₄alkoxy, nitro and amino;
Q is phenyl, a five-membered heterocyclic ring containing one, two, three or four heteroatoms chosen from O, N and S, at most one heteroatom being O or S, or a six-membered heterocyclic ring containing one, two or three nitrogen atoms, optionally substituted by C₁₋₄alkyl;
each R² and R³ is chosen from H and C₁₋₄alkyl, or R² and R³, together with the nitrogen atom to which they are attached, may form a six-membered ring optionally containing an oxygen atom or a further nitrogen atom, which ring is optionally substituted by C₁₋₄alkyl or Q; p is 1, 2 or 3; or a pharmaceutically acceptable salt thereof; pharmaceutical compositions comprising it; its use in methods of therapy; use of it for manufacturing medicaments; and methods of using it to treat diseases requiring administration of a VR1 antagonist such as pain, cough, GERD and depression.

The present invention is concerned with substituted nitrogen-containing six-membered amino-heterocycles and analogues and derivatives thereof as well as pharmaceutically acceptable salts thereof, which are useful as therapeutic compounds, particularly in the treatment of pain and other conditions ameliorated by the modulation of the function of the vanilloid-1 receptor (VR1).

The pharmacologically active ingredient of chilli peppers has been recognised for some time to be the phenolic amide capsaicin. The application of capsaicin to mucous membranes or when injected intradermally, causes intense burning-like pain in humans. The beneficial effects of topical administration of capsaicin as an analgesic is also well established. However, understanding of the underlying molecular pharmacology mediating these responses to capsaicin has been a more recent development.

The receptor for capsaicin, termed the vanilloid VR1 receptor, was cloned by Caterina and colleagues at UCSF in 1997 (Nature, 398:816, 1997). VR1 receptors are cation channels that are found on sensory nerves that innervate the skin, viscera, peripheral tissues and spinal cord. Activation of VR1 elicits action potentials in sensory fibres that ultimately generate the sensation of pain. Importantly the VR1 receptor is activated not only by capsaicin but also by acidic pH and by noxious heat stimuli. It is also sensitized by a number of inflammatory mediators and thus appears to be a polymodal integrator of painful stimuli.

The prototypical VR1 antagonist is capsazepine (Walpole et al., J. Med. Chem., 37:1942, 1994)—VR1 IC₅₀ of 420 nM. A novel series of sub-micromolar antagonists has also been reported recently (Lee et al, Bioorg. Med. Chem., 9:1713, 2001), but these reports provide no evidence for in vivo efficacy. A much higher affinity antagonist has been derived from the ‘ultra-potent’ agonist resiniferatoxin. Iodo-resiniferatoxin (Wahl et al., Mol. Pharmacol., 59:9, 2001) is a nanomolar antagonist of VR1 but does not possess properties suitable for an oral pharmaceutical. This last is also true of the micromolar peptoid antagonists described by Garcia-Martinez (Proc. Natl. Acad. Sci., USA, 99:2374, 2002). WO-A-0208221 has described a novel series of VR1 antagonists, which are stated to show efficacy in a number of animal models. We herein describe another novel series of VR1 modulators. These comprise predominantly VR1 antagonists but encompass VR1 partial antagonists and VR1 partial agonists. Such compounds have been shown to be efficacious in animal models of pain.

Related compounds are disclosed in WO-A-03099284 (Amgen Inc.). There is no disclosure of compounds in which Y is quinoline or isoquinoline. Preferred compounds of the present invention have improved pharmacokinetics with lower clearance and thus improved half-life.

The present invention provides compounds of formula I:
Y-J-L-Z (I)
wherein:

L is NR¹, O, S or CH₂;

J is a six-membered heterocycle containing one, two or three nitrogen atoms which is unsubstituted or substituted with up to three substituents, depending on the number of nitrogen atoms present, chosen independently from: halogen; hydroxy; nitro; cyano; isonitrile; C₃₋₇cycloalkyl; C₁₋₆alkyl; C₂₋₆alkenyl; C₂₋₆alkynyl; C₁₋₆alkoxy; C₃₋₇cycloalkoxy; hydroxyC₁₋₆alkyl; aminoC₁₋₆alkyl; C₁₋₆alkoxycarbonyl; haloC₁₋₆alkyl; haloC₁₋₆alkoxy; —NR²R³; —CONR²R³; —S(O)ₙC₁₋₆alkyl; —S(O)ₙNR²R³; —NHCOR¹; —NHS(O)ₙC₁₋₆alkyl; —COH, carboxy; —(CH₂)ₚNR²R³; —O(CH₂)_{q}NR²R³; —(CH₂)ₚQ; —O(CH₂)ₚQ; and phenyl, a five-membered heterocyclic ring containing one, two, three or four heteroatoms chosen from O, N and S, at most one heteroatom being O or S, or a six-membered heterocyclic ring containing one, two or three nitrogen atoms, wherein this substituent is unsubstituted or substituted by one, two or three groups chosen from halogen, hydroxy, nitro, cyano, isonitrile, C₁₋₆alkyl, C₁₋₆alkoxy, haloC₁₋₆alkyl, haloC₁₋₂alkoxy, —NR²R³, —CONR²R³, —S(O)ₙNR²R³, —NHCOR¹, NHS(O)ₙR¹, —COH, CO₂H and —S(O)ₙC₁₋₆alkyl;

when J is substituted by hydroxy, tautomerism may occur, in which case any nitrogen atom ortho or para to the resulting carbonyl group may be substituted as defined above;

Q is phenyl, a five-membered heterocyclic ring containing one, two, three or four heteroatoms chosen from O, N and S, at most one heteroatom being O or S, or a six-membered heterocyclic ring containing one, two or three nitrogen atoms, optionally substituted by halogen, C₁₋₄alkyl or haloC₁₋₄alkyl;

wherein J is substituted at positions meta to each other by L and Y;

Y is naphthalene or a fused 9- or 10-membered heteroaromatic system containing a six-membered heterocyclic ring, as defined above, or a phenyl ring, or a six-membered nitrogen-containing partially saturated ring, fused either to a six-membered heterocyclic ring as defined above or to a five-membered heterocyclic ring as defined above, Y being unsubstituted or substituted with one, two or three groups independently chosen from halogen, hydroxy, cyano, nitro, isonitrile, C₁₋₆alkyl, C₂₋₆alkenyl, C₂₋₆alkynyl, haloC₁₋₆alkyl, hydroxyC₁₋₆alkyl, aminoC₁₋₆alkyl, C₁₋₆alkoxy, C₁₋₆alkylthio, haloC₁₋₆alkoxy, —NR²R³, —CONR²R³, —S(O)ₙNR²R³, —(CH₂)ₚNR²R³, —NHCOR¹, NHS(O)ₙR¹, —COH, —CO₂H and C₁₋₆alkoxycarbonyl;

Z is phenyl, naphthyl, a six-membered heterocyclic ring containing one, two, or three nitrogen atoms or a five-membered heterocyclic ring containing one, two, three or four heteroatoms chosen from O, N and S, at most one heteroatom being O or S, Z being unsubstituted or substituted with one, two or three substituents independently chosen from halogen, hydroxy, cyano, nitro, isonitrile, C₁₋₆alkyl, C₂₋₆alkenyl, C₂₋₆alkynyl, haloC₁₋₆alkyl, hydroxyC₁₋₆alkyl, aminoC₁₋₆alkyl, C₁₋₆alkoxy, C₁₋₆alkylthio, haloC₁₋₆alkoxy, —NR²R³, —CONR²R³, —S(O)ₙNR²R³, —NHCOR¹, —NHS(O)ₙR¹, —COH, —CO₂H and C₁₋₆alkoxycarbonyl;

each R¹ is H or C₁₋₆alkyl;

each R² and R³ is chosen from H and C₁₋₆alkyl, or R² and R³, together with the nitrogen atom to which they are attached, may form a 4-6 membered ring optionally containing an oxygen atom or a further nitrogen atom, which ring is optionally substituted by C₁₋₆alkyl or Q;

each n is 0, 1 or 2;

each p is 1, 2, 3 or 4;

q is 2, 3 or 4;

or a pharmaceutically acceptable salt thereof.

L is preferably NR¹, particularly NH.

L may be CH₂ or NR¹.

J is preferably unsubstituted or substituted by one or two substituents. Most preferably J is unsubstituted or monosubstituted. J may be disubstituted.

J is thus preferably an unsubstituted or substituted pyrimidine, pyrazine, pyridazine or triazine. Pyrimidine is particularly favoured. J may be pyridine, which is unsubstituted or substituted, such as unsubstituted pyridine.

Substituents on J are preferably chosen independently from halogen, hydroxy, nitro, cyano, C₁₋₄alkyl, C₂₋₄alkenyl, C₂₋₄alkynyl, C₁₋₄alkoxy, C₁₋₄cycloalkoxy, hydroxyC₁₋₄alkyl, aminoC₁₋₄alkyl, haloC₁₋₄alkyl, haloC₁₋₄alkoxy, C₁₋₄alkoxycarbonyl, —NR²R³, C₁₋₄alkylthio, Q, CH₂Q, OCH₂Q, —(CH₂)ₚNR²R³, —CONR²R³ and —CO₂H. Substituents on J may be chosen independently from halogen, hydroxy, nitro, cyano, C₁₋₄alkyl, C₂₋₄alkenyl, C₂₋₄alkynyl, C₁₋₄alkoxy, hydroxyC₁₋₄alkyl, aminoC₁₋₄alkyl, haloC₁₋₄alkyl, haloC₁₋₄alkoxy, C₁₋₄alkoxycarbonyl, —NR²R³, —(CH₂)ₚNR²R³, —CONR²R³ and —CO₂H. In particular substituents are independently chosen from halogen, hydroxy, nitro, amino, C₁₋₄alkyl, haloC₁₋₄alkyl, C₃₋₅cycloalkyl and C₁₋₄alkoxy. Thus substituents can be chosen from chloro, fluoro, methyl, ethyl, isopropyl, cyclopropyl, trifluoromethyl, methoxy, nitro, amino, tertiarybutyl, hydroxymethyl, 2,6-dimethylmorpholino, bromo, methylthio, cyano, 2-methylpyrrolidino, morpholino, trifluoromethoxy, hydroxy, N-phenylpiperazinyl, 2,2,2-trifluoroethyl, morpholinomethyl, imidazomethyl, cyclopropylmethoxy, pyridomethoxy, morpholinoethoxy and tetrazolyl. Most preferably any substituents are chosen from chloro, fluoro, methyl, ethyl, isopropyl, cyclopropyl, trifluoromethyl, methoxy, nitro and amino.

More preferably any substituents are independently chosen from halogen, hydroxy, nitro, amino, C₁₋₄alkyl and C₁₋₄alkoxy. Most preferably the substituent is fluoro, methyl, methoxy, nitro or amino.

Particular embodiments of J are pyrimidin-2-yl, pyrazin-2-yl, pyridazin-3-yl, pyrimidin-4-yl, pyridazin-4-yl, 1,3,5-triazin-2-yl, 5-methoxypyrimidin-4-yl, 5-methylpyrimidin-4-yl, 5-fluoropyrimidin-4-yl, 2-methoxypyrimidin-4-yl, 2-methylpyrimidin-4-yl, 5-nitropyrimidin-4-yl and 5-aminopyrimidin-4-yl. Further particular embodiments of J are 5-tertiarybutylpyrimidin-4-yl, 2-trifluoromethylpyrimidin-4-yl, 2-hydroxymethylpyrimidin-4-yl, (cis-2,6-dimethylmorpholin-4-yl)methylpyrimidin-4-yl, 5-bromopyrimidin-4-yl, 2-methylthio-5-methylpyrimidin-4-yl, 2-cyano-5-methylpyrimidin-4-yl, 2-(2-methylpyrrolidin-1-yl)-5-methylpyrimidin-4-yl, 2-(morpholin-4-yl)-5-methylpyrimidin-4-yl, 2-(2,2,2-trifluoroethoxy)-5-methylpyrimidin-4-yl, 2-methyl-5-amninopyrimidin-4-yl, 2-hydroxypyrimidin-4-yl, 2-cyanopyrimidin-4-yl, 2-(morpholin-4-yl)pyrimidin-4-yl, 2-(1-phenylpiperazin-4-yl)pyrimidin-4-yl, 2-(2,2,2-trifluoroethyl)pyrimidin-4-yl, 2-methyltriazin-4-yl, 2-tertiarybutyl-5-methylpyrimidin-4-yl, 2-(morpholin-4-ylmethyl)pyrimidin-4-yl, 2-(imidazol-1-ylmethyl)pyrimidin-4-yl, 2-isopropyl-5-methylpyrimidin-4-yl, 2-methylthiopyrimidin-4-yl, 2-cyclopropylmethoxypyrimidin-4-yl, 2-(pyridine-3-ylmethoxyl)pyrimidin-4-yl, 2-trifluoromethyl-5-methylpyrimidin-4-yl, 2-(morpholin-4-ylethoxy)pyrimidin-4-yl and 2-(tetrazol-1-yl)pyrimidin-4-yl. For the avoidance of doubt the preceding lists indicate the position of attachment to L.

J may also be 2-chloropyrimidin-4-yl, 5-trifluoromethylpyridin-4-yl, 5-ethylpyrimidin-4-yl, 2-cyclopropyl-5-methylpyrimidin-4-yl, 5-isopropylpyrimidin-4-yl or pyridin-4-yl. p can be one or two.

Q can be pyridyl or phenyl. Q can be unsubstituted.

Y is thus preferably an unsubstituted or substituted quinoline, quinazoline, quinoxaline, phthalazine, isoquinoline, cinnoline, naphthyridine, indole, indazole, benzimidazole, benzothiazole, benzoxazole, imidazopyridine, imidazopyridazine, imidazopyrimidine, pyrazolopyridine, pyrazolopyridazine, pyrazolopyrimidine or triazolopyridine. Y may be substituted benzimidazole attached to J via the benzene portion. Y may be quinoxaline attached to L via the benzene portion. Y may be naphthyridine such as 1,8-naphthyridine or 1,5-naphthyridine. Y is most preferably an unsubstituted or substituted quinoline or isoquinoline, particularly a quinoline.

Substituents on Y are preferably independently chosen from halogen, hydroxy, cyano, nitro, amino, C₁₋₄alkyl, C₂₋₄alkenyl, C₂₋₄alkynyl, haloC₁₋₄alkyl, hydroxyC₁₋₄alkyl, aminoC₁₋₄alkyl, C₁₋₄alkoxy and haloC₁₋₄alkoxy. Particular substituents are hydroxy, halogen, C₁₋₄alkyl and haloC₁₋₄alkyl such as hydroxy, fluorine, methyl, ethyl and trifluoromethyl. The substituents can be halogen, C₁₋₄alkyl and haloC₁₋₄alkyl such as fluorine, methyl and trifluoromethyl.

Y is preferably unsubstituted or substituted with one or two substituents. More preferably Y is unsubstituted or monosubstituted. Y may be naphthalene or a fused 10-membered heteroaromatic ring. Y is generally a fused 10-membered heteroaromatic system.

Particular values of Y include quinolin-8-yl, quinoline-7-yl, 3-methylquinolin-7-yl, quinolin-5-yl, quinolin-6-yl, 6-fluoroquinolin-7-yl, 8-fluoroquinolin-7-yl, 6-trifluoromethylquinolin-7-yl, 8-fluoroquinolin-7-yl and isoquinolin-7-yl. Further particular values of Y include 8-ethylquinolin-7-yl, 1,8-naphthyridin-7-yl, 4-trifluoromethylquinolin-7-yl, 5-fluoroquinolin-7-yl, 1,5-naphthyridin-7-yl, 1-methyl-1H-benzimidazol-5-yl, 1H-benzimidazol-6-yl, 3-fluoroquinolin-7-yl, 4-hydroxyquinolin-7-yl and quinoxalin-6-yl.

Z is preferably a six-membered ring such as pyridazinyl, phenyl or pyridyl, preferably phenyl or pyridyl. Z is preferably monosubstituted, particular para to the attachment to L. Particular embodiments of Z include 4-trifluoromethylphenyl, 3-trifluoromethylpyrid-6-yl and 2-trifluoromethylpyrid-5-yl. Further embodiments include 4-trifluoromethoxyphenyl and 2-fluoro4-trifluoromethylphenyl. Yet further embodiments include 3-trifluoromethylpyridazin-6-yl and 3-fluoro-5-trifluoromethylpyridin-2-yl.

Substituents on Z are preferably independently chosen from halogen, amino, C₁₋₄alkyl, haloC₁₋₄alkyl, hydroxyC₁₋₄alkyl, aminoC₁₋₄alkyl, C₁₋₄alkoxy and haloC₁₋₄alkoxy. Particular substituents are haloC₁₋₄alkyl such as trifluoromethyl.

Z may be substituted by halogen, haloC₁₋₄alkyl or haloC₁₋₄alkoxy. Thus Z may be substituted by trifluoromethyl, trifluoromethoxy or fluorine.

Each R¹ is preferably hydrogen or C₁₋₄alkyl such as methyl. R¹ is particularly hydrogen.

Each R² and R³ is preferably independently hydrogen or C₁₋₄alkyl such as methyl. R² and R³ are preferably hydrogen. R² and R³ may form a piperidine, piperazine or morpholine ring, R² and R³ may then be substituted by C₁₋₄alkyl, phenyl or pyridyl, particularly when they form a piperazine ring.

A particularly preferred subclass of compounds is of formula Ia:
Y-J-NH-Z (Ia)
wherein:

Y is a quinoline or isoquinoline optionally substituted with one or two substituents independently chosen from hydroxy, halogen, haloC₁₋₄alkyl, C₁₋₄alkyl, C₁₋₄alkoxy, haloC₁₋₄alkoxy, nitro and amino;

J is pyridine, pyridazine, pyrazine, pyrimidine or triazine optionally substituted with one or two substituents independently chosen from hydroxy, halogen, haloC₁₋₄alkyl, C₁₋₄alkyl, C₃₋₅cycloalkyl, C₁₋₄alkoxy, hydroxyC₁₋₄alkyl, cyano, hydroxy, C₁₋₄cycloalkoxy, C₁₋₄alkylthio, haloC₁₋₄alkoxy, nitro, Q, (CH₂)ₚQ, —NR²R³, —(CH₂)ₚNR²R³ and —O(CH₂)ₚNR²R³;

wherein J is substituted at positions meta to each other by NH and Y; and

Z is phenyl or pyridyl optionally substituted with one or two substituents independently selected from halogen, haloC₁₋₄alkyl, C₁₋₄alkyl, C₁₋₄alkoxy, haloC₁₋₄alkoxy, nitro and amino;

Q is phenyl, a five-membered heterocyclic ring containing one, two, three or four heteroatoms chosen from O, N and S, at most one heteroatom being O or S, or a six-membered heterocyclic ring containing one, two or three nitrogen atoms, optionally substituted by C₁₋₄alkyl;

each R² and R³ is chosen from H and C₁₋₄alkyl, or R² and R³, together with the nitrogen atom to which they are attached, may form a six-membered ring optionally containing an oxygen atom or a further nitrogen atom, which ring is optionally substituted by C₁₋₄alkyl or Q;

p is 1, 2or 3;

or a pharmaceutically acceptable salt thereof.

In one embodiment:

Y is a quinoline or isoquinoline optionally substituted with one or two substituents independently chosen from halogen, haloC₁₋₄alkyl, C₁₋₄alkyl, C₁₋₄alkoxy, haloC₁₋₄alkoxy, nitro and amino;

J is pyridine, pyridazine, pyrazine, pyrimidine or triazine optionally substituted with one or two substituents independently chosen from halogen, haloC₁₋₄alkyl, C₁₋₄alkyl, C₃₋₅cycloalkyl, C₁₋₄alkoxy, haloC₁₋₄alkoxy, nitro and amino;

wherein J is substituted at positions meta to each other by NH and Y; and

Z is phenyl or pyridyl optionally substituted with one or two substituents independently selected from halogen, haloC₁₋₄alkyl, C₁₋₄alkyl, C₁₋₄alkoxy, haloC₁₋₄alkoxy, nitro and amino;

or a pharmaceutically acceptable salt thereof.

In another embodiment Y is a quinoline or isoquinoline optionally substituted with one or two substituents independently chosen from halogen, haloC₁₋₄alkyl, C₁₋₄alkyl, C₁₋₄alkoxy, haloC₁₋₄alkoxy, nitro and amino;

J is pyridazine, pyrazine, pyrimidine or triazine optionally substituted with one or two sugbstituents independently chosen from halogen, haloC₁₋₄alkyl, C₁₋₄alkyl, C₁₋₄alkoxy, haloC₁₋₄alkoxy, nitro and amino;

wherein J is substituted at positions meta to each other by NH and Y;

Z is phenyl or pyridyl optionally substituted with one or two substituents independently selected from halogen, haloC₁₋₄alkyl, C₁₋₄alkyl, C₁₋₄alkoxy, haloC₁₋₄alkoxy, nitro and amino;

or a pharmaceutically acceptable salt thereof.

Y is particularly quinoline or isoquinoline and is unsubstituted or monosubstituted. Preferred substituents include hydroxy, trifluoromethyl, fluorine, methyl and ethyl such as fluoro and methyl. Y may be quinoline.

J can be unsubstituted, monosubstituted or disubstituted with substituents preferably chosen from chloro, fluoro, methyl, ethyl, isopropyl, cyclopropyl, trifluoromethyl, methoxy, nitro, amino, tertiarybutyl, hydroxymethyl, 2,6-dimethylmorpholino, bromo, methylthio, cyano, 2-methylpyrrolidino, morpholino, trifluoromethoxy, hydroxy, N-phenylpiperazinyl, 2,2,2-trifluoroethyl, morpholinomethyl, imidazomethyl, cyclopropylmethoxy, pyridomethoxy, morpholinoethoxy and tetrazolyl. The substituents are preferably chloro, fluoro, methyl, ethyl, isopropyl, cyclopropyl, trifluoromethyl, methoxy, nitro and amino.

J is preferably unsubstituted or monosubstituted with fluorine, methoxy, methyl, amino or nitro. J is preferably pyrimidine. J may be pyridine. J may be triazine.

Z is preferably monosubstituted at a position para to the point of attachment to NH. Z may be substituted by F, CF₃ or OCF₃. The substituent is preferably CF₃.

Particularly preferred are compounds of formula Ia in which:

Y is a quinoline or isoquinoline optionally substituted with one or two substituents independently chosen from halogen, haloC₁₋₄alkyl, C₁₋₄alkyl, C₁₋₄alkoxy, haloC₁₋₄alkoxy, nitro and amino;

J is pyrimidine optionally substituted with one or two substituents independently chosen from halogen, haloC₁₋₄alkyl, C₁₋₄alkyl, C₃₋₅cycloalkyl, C₁₋₄alkoxy, haloC₁₋₄alkoxy, nitro and amino;

wherein J is substituted at positions meta to each other by NH and Y; and

Z is pyridyl substituted at at least the position para to the point of attachment to NH by CF₃ or OCF₃ and which is optionally further substituted by halogen;

or a pharmaceutically acceptable salt thereof.

Particular embodiments of Y, J and Z are described above.

Particular embodiments of the invention include:
4-quinolin-8-yl-N-[4-trifluoromethylphenyl]pyrimidin-2-amine;
6-quinolin-8-yl-N-[4-trifluoromethylphenyl]pyrazin-2-amine;
5-quinolin-8-yl-N-[4-trifluoromethylphenyl]pyridazin-3-amine;
6-quinolin-8-yl-N-[4-trifluoromethylphenyl]pyrimidin-4-amine;
6-quinolin-7-yl-N-[4-trifluoromethylphenyl]pyrazin-2-amine;
4-quinolin-7-yl-N-[4-trifluoromethylphenyl]pyrimidin-2-amine;
6-quinolin-7-yl-N-[4-trifluoromethylphenyl]pyrimidin-4-amine;
5-quinolin-7-yl-N-[4-trifluoromethylphenyl]pyridazin-3-amine;
6-quinolin-7-yl-N-[5-trifluoromethylpyridin-2-yl]pyrimidin-4-amine;
6-quinolin-7-yl-N-[6-trifluoromethylpyridin-3-yl]pyrimidin-4-amine;
5-methoxy-6-quinolin-7-yl-N-[4-trifluoromethylphenyl]pyrimidin-4-amine;
5-methyl-6-quinolin-7-yl-N-[4-trifluoromethylphenyl]pyrimidin-4-amine;
5-fluoro-6-quinolin-7-yl-N-[4-trifluoromethylphenyl]pyrimidin-4-amine;
2-methoxy-6-quinolin-7-yl-N-[4-trifluoromethylphenyl]pyrimidin-4-amine;
2-methyl-6-quinolin-7-yl-N-[4-trifluoromethylphenyl]pyrimidin-4-amine;
6-(3-methylquinolin-7-yl)-N-[4-trifluoromethylphenyl]pyrimidin-4-amine;
6-quinolin-5-yl-N-[4-trifluoromethylphenyl]pyrimidin-4-amine;
6-quinolin-6-yl-N-[4-trifluoromethylphenyl]pyrimidin-4-amine;
6-(2-methylquinolin-7-yl)-N-[4-trifluoromethylphenyl]pyrimidin-4-amine;
6-(6-fluoroquinolin-7-yl)-N-[4-trifluoromethylphenyl]pyrimidin-4-amine;
6-(8-fluoroquinolin-7-yl)-N-[4-trifluoromethylphenyl]pyrimidin-4-amine;

N-[4-(trifluoromethyl)phenyl]-6-[6-trifluoromethylquinolin-7-yl]pyrimidin-4-amine;
6-(8-methylquinolin-7-yl)-N-[4-trifluoromethylphenyl]pyrimidin-4-amine;
5-fluoro-6-(8-methylquinolin-7-yl)-N-[4-trifluoromethylphenyl]pyrimidin-4-amine;
6-isoquinolin-7-yl-N-[4-trifluoromethylphenyl]pyrimidin-4-amine;
6-quinolin-8-yl-N-[4-trifluoromethylphenyl]pyridazin-4-amine;
4-quinolin-8-yl-N-[4-trifluoromethylphenyl]-1,3,5-triazin-2-amine;
5-nitro-6-quinolin-7-yl-N-[4-trifluoromethylphenyl]pyrimidin-4-amine;
6-quinolin-7-yl-N⁴-[4-trifluoromethylphenyl]pyrimidin-4,5-diamine;
and the pharmaceutically acceptable salts thereof.

Further particular embodiments include:
6-(8-fluoroquinolin-7-yl)-5-methyl-N-[5-trifluoromethylpyridin-2-yl]pyrimidin-4-amine;
6-(8-fluoroquinolin-7-yl)-5-methyl-N-[4-trifluoromethylphenyl]pyrimidin-4-amine;
5-methoxy-2-methyl-6-quinolin-7-yl-N-[4-trifluoromethylphenyl]pyrimidin-4-amine;
2-methyl-6-(8-methylquinolin-7-yl)-N-[4-trifluoromethylphenyl]pyrimidin-4-amine;
N-[2-fluoro-4-trifluoromethylphenyl]-5-methoxy-6-quinolin-7-ylpyrimidin-4-amine;
5-methoxy-6-quinolin-7-yl-N-[4-trifluoromethoxyphenyl]pyrimidin-4-amine;
5-methyl-6-(8-methylquinolin-7-yl)-N-[4-trifluoromethylphenyl]pyrimidin-4-amine;
5-methyl-6-(8-methylquinolin-7-yl)-N-[5-trifluoromethylpyridin-2-yl]pyrimidin-4-amine;
6-quinolin-7-yl-5-trifluoromethyl-N-[4-trifluoromethylphenyl]pyrimidin-4-amine;
5-ethyl-6-quinolin-7-yl-N-[4-trifluoromethylphenyl]pyrimidin-4-amine;
5-ethyl-6-quinolin-7-yl-N-[5-trifluoromethylpyridin-2-yl]pyrimidin-4-amine;
5-methyl-6-quinolin-7-yl-N-[5-trifluoromethylpyridin-2-yl]pyrimidin-4-amine;
2-cyclopropyl-5-methyl-6-quinolin-7-yl-N-[5-trifluoromethylpyridin-2-yl]pyrimidin-4-amine;
2-cyclopropyl-5-methyl-6-quinolin-7-yl-N-[4-trifluoromethylphenyl]pyrimidin-4-amine;
5-isopropyl-6-quinolin-7-yl-N-[5-trifluoromethylpyridin-2-yl]pyrimidin-4-amine;
6-(6-fluoroquinolin-7-yl)-5-methyl-N-[5-trifluoromethylpyridin-2-yl]pyrimidin-4-amine;
6-(6-fluoroquinolin-7-yl)-5-methyl-N-[4-trifluoromethylphenyl]pyrimidin-4-amine;
5-fluoro-6-(8-methylquinolin-7-yl)-N-[5-trifluoromethylpyridin-2-yl]pyrimidin-4-amine;
N-(2-quinolin-7-ylpyridin-4-yl)-5-trifluoromethylpyridin-2-amine;
2-quinolin-7-yl-N-[4-trifluoromethylphenyl]pyridin-4-amine;
5-chloro-6-quinolin-7-yl-N-[4-trifluoromethylphenyl]pyrimidin-4-amine;
5-chloro-6-quinolin-7-yl-N-[5-trifluoromethylpyridin-2-yl]pyrimidin-4-amine;

and the pharmaceutically acceptable salts thereof.

Further preferred embodiments include:
5-tert-butyl-6-quinolin-7-yl-N-[4-trifluoromethylphenyl]pyrimidin-4-amine;
5-tert-butyl-6-quinolin-7-yl-N-[5-trifluoromethylpyridin-2-yl]pyrimidin-4-amine;
6-(8-ethylquinolin-7-yl)-N-[4-trifluoromethylphenyl]pyrimidin-4-amine;
6-(8-ethylquinolin-7-yl)-N-[5-trifluoromethylpyridin-2-yl]pyrimidin-4-amine;
6-(8-ethylquinolin-7-yl)-5-methyl-N-[4-trifluoromethylphenyl]pyrimidin-4-amine;
6-(8-ethylquinolin-7-yl)-5-methyl-N-[5-trifluoromethylpyridin-2-yl]pyrimidin-4-amine;
N-[2-fluoro-4-trifluoromethylphenyl]-5-methyl-6-quinolin-7-ylpyrimidin-4-amine;
6-(8-methylquinolin-7-yl)-2-trifluoromethyl-N-[4-trifluoromethylphenyl]pyrimidin-4-amine;
6-(8-methylquinolin-7-yl)-2-trifluoromethyl-N-[5-trifluoromethylpyridin-2-yl]pyrimidin-4-amine;
2-methoxymethyl-5-methyl-6-quinolin-7-yl-N-[5-trifluoromethylpyridin-2-yl]pyrimidin-4-amine;
5-fluoro-6-(8-fluoroquinolin-7-yl)-N-[4-trifluoromethylphenyl]pyrimidin-4-amine;
5-fluoro-6-(8-fluoroquinolin-7-yl)-N-[5-trifluoromethylpyridin-2-yl]pyrimidin-4-amine;
N-(5-methyl-6-quinolin-7-ylpyrimidin-4-yl)-6-trifluoromethylpyridazin-3-amine;
6-(1,8-naphthyridin-2-yl)-N-[4-trifluoromethylphenyl]pyrimidin-4-amine;
5-methyl-6-quinolin-8-yl-N-[4-trifluoromethylphenyl]pyrimidin-4-amine;
5-methyl-6-quinolin-8-yl-N-[5-trifluoromethylpyridin-2-yl]pyrimidin-4-amine;
N-[4-trifluoromethylphenyl]-6-[4-trifluoromethylquinolin-7-yl]pyrimidin-4-amine;
5-methyl-N-[4-trifluoromethylphenyl]-6-[4-trifluoromethylquinolin-7-yl]pyrimidin-4-amine;
5-methyl-N-[5-trifluoromethylpyridin-2-yl]-6-[4-trifluoromethylquinolin-7-yl]pyrimidin-4-amine;
6-quinolin-7-yl-N⁴-[5-trifluoromethylpyridin-2-yl]pyrimidine-4,5-diamine;
N-[3-fluoro-5-trifluoromethylpyridin-2-yl]-5-methyl-6-quinolin-7-ylpyrimidin-4-amine;
(5-methyl-4-quinolin-7-yl-6-{5-trifluoromethylpyridin-2-ylamino}pyrimidin-2-yl)methanol;
2-[(cis-2,6-dimethylmorpholin-4-yl)methyl]-5-methyl-6-quinolin-7-yl-N-[5-trifluoromethylpyridin-2-yl]pyrimidin-4-amine;
5-methyl-6-(1,8-naphthyridin-2-yl)-N-[4-trifluoromethylphenyl]pyrimidin-4-amine;
5-methyl-6-(1,8-naphthyridin-2-yl)-N-[5-trifluoromethylpyridin-2-yl]pyrimidin-4-amine;
5-isopropyl-6-(1,8-naphthyridin-2-yl)-N-[4-trifluoromethylphenyl]pyrimidin-4-amine;
5-tert-butyl-6-(1,8-naphthyridin-2-yl)-N-[4-trifluoromethylphenyl]pyrimidin-4-amine;
6-(5-fluoroquinolin-7-yl)-5-methyl-N-[5-trifluoromethylpyridin-2-yl]pyrimidin-4-amine;
5-methyl-6-(1,5-naphthyridin-3-yl)-N-[4-trifluoromethylphenyl]pyrimidin-4-amine;
5-methyl-6-(1,5-naphthyridin-3-yl)-N-[5-trifluoromethylpyridin-2-yl]pyrimidin-4-amine;
5-methyl-6-(1-methyl-1H-benzimidazol-5-yl)-N-[4-trifluoromethylphenyl]pyrimidin-4-amine;
6-(1H-benzimidazol-6-yl)-N-[4-trifluoromethylphenyl]pyrimidin-4-amine;
5-bromo-6-quinolin-7-yl-N-[4-trifluoromethylphenyl]pyrimidin-4-amine;
5-methyl-2-methylthio-6-quinolin-7-yl-N-[4-trifluoromethylphenyl]pyrimidin-4-amine;
5-methyl-4-quinolin-7-yl-6-{4-trifluoromethylphenylamino}pyrimidine-2-carbonitrile;
6-(3-fluoroquinolin-7-yl)-5-methyl-N-[5-trifluoromethylpyridin-2-yl]pyrimidin-4-amine;
5-methyl-2-(2-methylpyrrolidin-1-yl)-6-quinolin-7-yl-N-[5-trifluoromethylpyridin-2-yl]pyrimidin-4-amine;
5-methyl-2-morpholin-4-yl-6-quinolin-7-yl-N-[5-trifluoromethylpyridin-2-yl]pyrimidin-4-amine;
5-methyl-6-quinolin-7-yl-2-(2,2,2-trifluoroethoxy)-N-[5-trifluoromethylpyridin-2-yl]pyrimidin-4-amine;
7-(5-methyl-6-{5-trifluoromethylpyridin-2-ylamino}pyrimidin-4-yl)quinolin-4-ol;
5-5-methyl-6-{5-trifluoromethylpyridin-2-ylamino}pyrimidin-4-ylquinolin-4-ol;
2-methyl-6-quinolin-7-yl-N⁴-[4-trifluoromethylphenyl]pyrimidine-4,5-diamine;
4-quinolin-7-yl-6-{4-trifluoromethylphenylamino}pyrimidin-2-ol;
2-chloro-6-quinolin-7-yl-N-[4-trifluoromethylphenyl]pyrimidin-4-amine;
2-morpholin-4-yl-6-quinolin-7-yl-N-[4-trifluoromethylphenyl]pyrimidin-4-amine;
2-(4-phenylpiperazin-1-yl)-6-quinolin-7-yl-N-[4-trifluoromethylphenyl]pyrimidin-4-amine;
6-quinolin-7-yl-N²-(2,2,2-trifluoroethyl)-N⁴-[4-trifluoromethylphenyl]pyrimidine-2,4-diamine;
4-methyl-6-quinolin-7-yl-N-[4-trifluoromethylphenyl]-1,3,5-triazin-2-amine;
2-(1,1-dimethylethyl)-5-methyl-6-quinolin-7-yl-N-[4-trifluoromethylphenyl]pyrimidin-4-amine;
5-methyl-6-quinolin-5-yl-N-[5-trifluoromethylpyridin-2-yl]pyrimidin-4-amine;
2-(morpholin-4-ylmethyl)-6-quinolin-7-yl-N-[4-trifluoromethylphenyl]pyrimidin-4-amine;
(4-quinolin-7-yl-6-{4-trifluoromethylphenylamino}pyrimidin-2-yl)methanol;
2-(1H-imidazol-1-ylmethyl)-6-quinolin-7-yl-N-[4-trifluoromethylphenyl]pyrimidin-4-amine;
2-isopropyl-5-methyl-6-quinolin-7-yl-N-[4-trifluoromethyl phenyl]pyrimidin-4-amine;
2-methylthio-6-quinolin-7-yl-N-[4-trifluoromethylphenyl]pyrimidin-4-amine;
4-quinolin-7-yl-6-{4-trifluoromethylphenylamino}pyrimidine-2-carbonitrile;
2-cyclopropylmethoxy-6-quinolin-7-yl-N-[4-trifluoromethylphenyl]-pyrimidin-4-amine;
2-(pyridin-3-ylmethoxy)-6-quinolin-7-yl-N-[4-trifluoromethylphenyl]-pyrimidin-4-amine;
2-(2-morpholin-4-ylethoxy)-6-quinolin-7-yl-N-[4-trifluoromethylphenyl]-pyrimidin-4-amine;
6-quinolin-7-yl-2-(1H-tetrazol-5-yl)-N-[4-trifluoromethylphenyl]-pyrimidin-4-amine trifluoroacetic acid salt;
6-quinolin-7-yl-2-trifluoromethyl-N-[4-trifluoromethylphenyl]-pyrimidin-4-amine;
6-quinoxalin-6-yl-N-[4-trifluoromethylphenyl]pyrimidin-4-amine;
5-methyl-6-quinoxalin-6-yl-N-[4-trifluoromethylphenyl]pyrimidin-4-amine;
5-methyl-6-quinolin-7-yl-2-trifluoromethyl-N-[4-trifluoromethylphenyl]pyrimidin-4-amine;
5-methyl-2-methylthio-6-quinolin-7-yl-N-[5-trifluoromethylpyridin-2-yl]pyrimidin-4-amine;
5-methyl-2-methylsulfonyl-6-quinolin-7-yl-N-[4-trifluoromethylphenyl]pyrimidin-4-amine;
5-methyl-2-methylsulfonyl-6-quinolin-7-yl-N-[5-trifluoromethylpyridin-2-yl]pyrimidin-4-amine;
2-methylsulfonyl-6-quinolin-7-yl-N-[4-trifluoromethylphenyl]pyrimidin-4-amine;

and the pharmaceutically acceptable salts thereof.

Preferred pharmaceutically acceptable salts are the besylate salts.

Further particular embodiments include:
7-(5-methyl-6-{5-trifluoromethylpyridin-2-ylamino}pyrimidin-4-yl)quinolinium benzenesulfonate;
7-(2-cyclopropyl-5-methyl-6-{5-trifluoromethylpyridin-2-ylamino}pyrimidin-4-yl)quinolinium benzenesulfonate;
7-(2-cyclopropyl-5-methyl-6-{5-trifluoromethylphenylamino}pyrimidin-4-yl)quinolinium benzenesulfonate;
7-(5-isopropyl-6-{5-trifluoromethylpyridin-2-ylamino}pyrimidin-4-yl)quinolinium benzenesulfonate;
6-fluoro-7-(5-methyl-6-{5-trifluoromethylpyridin-2-ylamino}pyrimidin-4-yl)quinolinium benzenesulfonate;
6-fluoro-7-(5-methyl-6-{4-trifluoromethylphenylamino}pyrimidin-4-yl)quinolinium benzenesulfonate.

As used herein, the term “alkyl” or “alkoxy” as a group or part of a group means that the group is straight or branched. Examples of suitable alkyl groups include methyl, ethyl, n-propyl, i-propyl, n-butyl, s-butyl and t-butyl. Examples of suitable alkoxy groups include methoxy, ethoxy, n-propoxy, i-propoxy, n-butoxy, s-butoxy and t-butoxy. “Alkylthio” shall be construed in an analogous manner. Examples of “C₃₋₇cycloalkyl” groups are cyclohexyl, cyclopentyl, cyclobutyl, cyclopropyl and methylcyclopropyl groups.

As used herein, the term “hydroxyC₁₋₆alkyl” means a C₁₋₆alkyl group in which one or more (in particular 1 to 3, and especially 1) hydrogen atoms have been replaced by hydroxy groups. Particularly preferred are hydroxyC₁₋₃alkyl groups, for example, CH₂OH, CH₂CH₂OH, CH(CH₃)OH or C(CH₃)₂OH, and most especially CH₂OH. “AminoC₁₋₆alkyl” shall be construed in an analogous manner.

As used herein, the terms “haloC₁₋₆alkyl” and “haloC₁₋₆alkoxy” means a C₁₋₆alkyl or C₁₋₆alkoxy group in which one or more (in particular, 1 to 3) hydrogen atoms have been replaced by halogen atoms, especially fluorine or chlorine atoms. Preferred are fluoroC₁₋₆alkyl and fluoroC₁₋₆alkoxy groups, in particular, fluoroC₁₋₃alkyl and fluoroC₁₋₃alkoxy groups, for example, CF₃, CH₂CH₂F, CH₂CHF₂, CH₂CF₃, OCF₃, OCH₂CH₂F, OCH₂CHF₂ or OCH₂CF₃, and most especially CF₃ and OCF₃.

As used herein, the terms “alkenyl” and “alkynyl” as a group or part of a group means that the group is straight or branched. Examples of suitable alkenyl groups include vinyl and allyl. A suitable alkynyl group is acetylene or propargyl.

When used herein, the term “halogen” means fluorine, chlorine, bromine and iodine. The most preferred halogens are fluorine and chlorine, especially fluorine.

When used herein, the term “C₁₋₆alkoxycarbonyl” denotes a C₁₋₆alkoxy radical attached via the oxygen atom thereof to a carbonyl (C═O) radical thus forming a C₁₋₆alkoxycarbonyl or haloC₁₋₆alkoxycarbonyl radical. Suitable examples of such esterified carboxy groups include, for example, methoxycarbonyl, ethoxycarbonyl, propoxycarbonyl, isopropoxycarbonyl and tert-butoxycarbonyl.

Examples of 6-membered heterocycles are pyridine, pyrimidine, pyrazine, pyridazine and triazine.

Examples of 5-membered heterocycles are thiophene, furan, pyrrole, imidazole, pyrazole, oxazole, isoxazole, thiazole, isothiazole, 1,2,3-triazole, 1,2,4-triazole, oxadiazole, thiadiazole and tetrazole.

“Heterocyclic” in the above is interchangeable with “heteroaromatic”.

In a further aspect of the present invention, the compounds of formula I may be prepared in the form of a pharmaceutically acceptable salt, especially an acid addition salt.

For use in medicine, the salts of the compounds of formula I will be non-toxic pharmaceutically acceptable salts. Other salts may, however, be useful in the preparation of the compounds according to the invention or of their non-toxic pharmaceutically acceptable salts. Suitable pharmaceutically acceptable salts of the compounds of this invention include acid addition salts which may, for example, be formed by mixing a solution of the compound according to the invention with a solution of a pharmaceutically acceptable acid such as hydrochloric acid, fumaric acid, p-toluenesulphonic acid, maleic acid, succinic acid, acetic acid, citric acid, tartaric acid, carbonic acid, phosphoric acid or sulphuric acid. A further salt is the acid addition salt with benzenesulfonic acid. Preferred pharmaceutically acceptable salts of the compounds of the present invention are the besylate salts. The hydrochloride salt can also be used. Salts of amine groups may also comprise quaternary ammonium salts in which the amino nitrogen atom carries a suitable organic group such as an alkyl, alkenyl, alkynyl or aralkyl moiety. Furthermore, where the compounds of the invention carry an acidic moiety, suitable pharmaceutically acceptable salts thereof may include metal salts such as alkali metal salts, e.g. sodium or potassium salts; and alkaline earth metal salts, e.g. calcium or magnesium salts.

The salts may be formed by conventional means, such as by reacting the free base form of the compound of formula I with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

The present invention also includes within its scope N-oxides of the compounds of formula I above. In general, such N-oxides may be formed on any available nitrogen atom. The N-oxides may be formed by conventional means, such as reacting the compound of formula I with oxone in the presence of wet alumina.

The present invention includes within its scope prodrugs of the compounds of formula I above. In general, such prodrugs will be functional derivatives of the compounds of formula I which are readily convertible in vivo into the required compound of formula I. Conventional procedures for the selection and preparation of suitable prodrug derivatives are described, for example, in “Design of Prodrugs”, ed. H. Bundgaard, Elsevier, 1985.

A prodrug may be a pharmacologically inactive derivative of a biologically active substance (the “parent drug” or “parent molecule”) that requires transformation within the body in order to release the active drug, and that has improved delivery properties over the parent drug molecule. The transformation in vivo may be, for example, as the result of some metabolic process, such as chemical or enzymatic hydrolysis of a carboxylic, phosphoric or sulphate ester, or reduction or oxidation of a susceptible functionality.

The present invention includes within its scope solvates of the compounds of formula I and salts thereof, for example, hydrates.

The compounds according to the invention may have one or more asymmetric centres, and may accordingly exist both as enantiomers and as diastereoisomers. It is to be understood that all such isomers and mixtures thereof are encompassed within the scope of the present invention. Furthermore, the compounds of formula I may also exist in tautomeric forms and the invention includes within its scope both mixtures and separate individual tautomers.

The present invention further provides pharmaceutical compositions comprising one or more compounds of formula I in association with a pharmaceutically acceptable carrier or excipient.

Preferably the compositions according to the invention are in unit dosage forms such as tablets, pills, capsules, powders, granules, sterile parenteral solutions or suspensions, metered aerosol or liquid sprays, drops, ampoules, auto-injector devices, suppositories, creams or gels; for oral, parenteral, intrathecal, intranasal, sublingual, rectal or topical administration, or for administration by inhalation or insufflation. Oral compositions such as tablets, pills, capsules or wafers are particularly preferred. For preparing solid compositions such as tablets, the principal active ingredient is mixed with a pharmaceutical carrier, e.g. conventional tabletting ingredients such as corn starch, lactose, sucrose, sorbitol, talc, stearic acid, magnesium stearate, dicalcium phosphate or gums, and other pharmaceutical diluents, e.g. water, to form a solid pre-formulation composition containing a homogeneous mixture of a compound of the present invention, or a pharmaceutically acceptable salt thereof. When referring to these pre-formulation compositions as homogeneous, it is meant that the active ingredient is dispersed evenly throughout the composition so that the composition may be readily subdivided into equally effective unit dosage forms such as tablets, pills and capsules. This solid pre-formulation composition is then subdivided into unit dosage forms of the type described above containing from 0.1 to about 500 mg of the active ingredient of the present invention. Favoured unit dosage forms contain from 1 to 500 mg, for example 1, 5, 10, 25, 50, 100, 300 or 500 mg, of the active ingredient. The tablets or pills of the novel composition can be coated or otherwise compounded to provide a dosage form affording the advantage of prolonged action. For example, the tablet or pill can comprise an inner dosage and an outer dosage component, the latter being in the form of an envelope over the former. The two components can be separated by an enteric layer that serves to resist disintegration in the stomach and permits the inner component to pass intact into the duodenum or to be delayed in release. A variety of materials can be used for such enteric layers or coatings, such materials including a number of polymeric acids and mixtures of polymeric acids with such materials as shellac, cetyl alcohol and cellulose acetate.

The liquid forms in which the novel compositions of the present invention may be incorporated for administration orally or by injection include aqueous solutions, suitably flavoured syrups, aqueous or oil suspensions, and flavoured emulsions with edible oils such as cottonseed oil, sesame oil, coconut oil or peanut oil, as well as elixirs and similar pharmaceutical vehicles. Suitable dispersing or suspending agents for aqueous suspensions include synthetic and natural gums such as tragacanth, acacia, alginate, dextran, sodium carboxymethylcellulose, methylcellulose, polyvinyl-pyrrolidone or gelatin.

In the treatment of painful conditions such as those listed below, a suitable dosage level is about 1.0 mg to 15 g per day, preferably about 5.0 mg to 1 g per day, more preferably about 5 mg to 500 mg per day, especially 10 mg to 100 mg per day. The compounds may be administered on a regimen of 1 to 4 times per day.

It will be appreciated that the amount of a compound of formula I required for use in any treatment will vary not only with the particular compounds or composition selected but also with the route of administration, the nature of the condition being treated, and the age and condition of the patient, and will ultimately be at the discretion of the attendant physician.

The invention further provides a compound of formula I as defined above, or a pharmaceutically acceptable salt thereof, for use in treatment of the human or animal body. Preferably, said treatment is for a condition which is susceptible to treatment by modulation (preferably antagonism) of VR1 receptors.

The compounds of the present invention will be of use in the prevention or treatment of diseases and conditions in which pain and/or inflammation predominates, including chronic and acute pain conditions. Such conditions include rheumatoid arthritis; osteoarthritis; post-surgical pain; musculo-skeletal pain, particularly after trauma; spinal pain; myofascial pain syndromes; headache, including migraine, acute or chronic tension headache, cluster headache, temporomandibular pain, and maxillary sinus pain; ear pain; episiotomy pain; burns, and especially primary hyperalgesia associated therewith; deep and visceral pain, such as heart pain, muscle pain, eye pain, orofacial pain, for example, odontalgia, abdominal pain, gynaecological pain, for example, dysmenorrhoea, pain associated with cystitis and labour pain, chronic pelvic pain, chronic prostatitis and endometriosis; pain associated with nerve and root damage, such as pain associated with peripheral nerve disorders, for example, nerve entrapment and brachial plexus avulsions, amputation, peripheral neuropathies, tic douloureux, atypical facial pain, nerve root damage, and arachnoiditis; itching conditions including pruritis, itch due to hemodialysis, and contact dermatitis; pain (as well as broncho-constriction and inflammation) due to exposure (e.g. via ingestion, inhalation, or eye contact) of mucous membranes to capsaicin and related irritants such as tear gas, hot peppers or pepper spray; neuropathic pain conditions such as diabetic neuropathy, chemotherapy-induced neuropathy and post-herpetic neuralgia; “non-painful” neuropathies; complex regional pain syndromes; pain associated with carcinoma, often referred to as cancer pain; central nervous system pain, such as pain due to spinal cord or brain stem damage, low back pain, sciatica and ankylosing spondylitis; gout; scar pain; irritable bowel syndrome; inflammatory bowel disease; urinary incontinence including bladder detrusor hyper-reflexia and bladder hypersensitivity; respiratory diseases including chronic obstructive pulmonary disease (COPD), chronic bronchitis, cystic fibrosis, asthma and rhinitis, including allergic rhinitis such as seasonal and perennial rhinitis, and non-allergic rhinitis; autoimmune diseases; and immunodeficiency disorders. In particular conditions that can be treated or prevented by the compounds of the present invention include respiratory diseases such as chronic obstructive pulmonary disease (COPD); chronic bronchitis; cystic fibrosis; asthma; and rhinitis, including allergic rhinitis such as seasonal and perennial rhinitis, non-allergic rhinitis and cough. The compounds of the present invention may also be useful in the treatment of depression. They may also be used to treat gastro-oesophageal reflux disease (GERD), particularly the pain associated with GERD.

Thus, according to a further aspect, the present invention provides a compound of formula I for use in the manufacture of a medicament for the treatment or prevention of physiological disorders that may be ameliorated by modulating VR1 activity.

The present invention also provides a method for the treatment or prevention of physiological disorders that may be ameliorated by modulating VR1 activity, which method comprises administration to a patient in need thereof of an effective amount of a compound of formula I or a composition comprising a compound of formula I.

According to a further or alternative aspect, the present invention provides a compound of formula I for use in the manufacture of a medicament for the treatment or prevention of a disease or condition in which pain and/or inflammation predominates.

According to a further alternative aspect, the present invention provides a compound of formula I for use in the manufacture of a medicament for the treatment or prevention of respiratory diseases such as cough.

The present invention also provides a method for the treatment or prevention of a disease or condition in which pain and/or inflammation predominates, which method comprises administration to a patient in need thereof of an effective amount of a compound of formula I or a composition comprising a compound of formula I.

The present invention also provides a method for the treatment or prevention of respiratory diseases, such as cough, which method comprises administration to a patient in need thereof of an effective amount of a compound of formula I or a composition comprising a compound of formula I.

According to a further aspect of the present invention, it may be desirable to treat any of the aforementioned conditions with a combination of a compound according to the present invention and one or more other pharmacologically active agents suitable for the treatment of the specific condition. The compound of formula I and the other pharmacologically active agent(s) may be administered to a patient simultaneously, sequentially or in combination.

Thus, for example, for the treatment or prevention of pain and/or inflammation, a compound of the present invention may be used in conjunction with other analgesics, such as acetaminophen (paracetamol), aspirin and other NSAIDs, including selective cyclooxygenase-2 (COX-2) inhibitors, as well as opioid analgesics, especially morphine, NR2B antagonists, bradykinin antagonists, anti-migraine agents, anticonvulsants such as oxcarbazepine and carbamazepine, antidepressants (such as TCAs, SSRIs, SNRIs, substance P antagonists, etc.), spinal blocks, gabapentin, pregabalin and asthma treatments (such as θ₂-adrenergic receptor agonists or leukotriene D₄antagonists (e.g. montelukast).

Specific anti-inflammatory agents include diclofenac, ibuprofen, indomethacin, nabumetone, ketoprofen, naproxen, piroxicam and sulindac, etodolac, meloxicam, rofecoxib, celecoxib, etoricoxib, parecoxib, valdecoxib and tilicoxib. Suitable opioid analgesics of use in conjunction with a compound of the present invention include morphine, codeine, dihydrocodeine, diacetylmorphine, hydrocodone, hydromorphone, levorphanol, oxymorphone, alfentanil, buprenorphine, butorphanol, fentanyl, sufentanyl, meperidine, methadone, nalbuphine, propoxyphene and pentazocine; or a pharmaceutically acceptable salt thereof. Suitable anti-migraine agents of use in conjunction with a compound of the present invention include CGRP-antagonists, ergotamines or 5-HT₁ agonists, especially sumatriptan, naratriptan, zolmatriptan or rizatriptan.

Therefore, in a further aspect of the present invention, there is provided a pharmaceutical composition comprising a compound of the present invention and an analgesic, together with at least one pharmaceutically acceptable carrier or excipient.

In a further or alternative aspect of the present invention, there is provided a product comprising a compound of the present invention and an analgesic as a combined preparation for simultaneous, separate or sequential use in the treatment or prevention of a disease or condition in which pain and/or inflammation predominates.

The compounds of formula I can be made by reacting a compound of formula II with a compound of formula III:
Y-J-L¹ (II)
HL-Z (III)
wherein J, L, Y and Z are as defined above and L¹ is a leaving group such as chlorine. The reaction can be carried out in the presence of a base such as sodium tertiarybutoxide or sodium hydrogencarbonate and a coupling agent such as 2′-(dimethylamino)-2-biphenylyl palladium (II) chloride dinorbomylphosphine complex generally in a solvent such as toluene or tetrahydrofuran with heating to reflux for several hours to several days. Alternatively the reaction can be carried out in the presence of cesium carbonate, a coupling agent such as 4,5-bis(diphenylphosphino)-9,9-dimethylxanthene and a catalyst such as Pd₂(dba)₃, generally in a solvent such as anhydrous dioxane at reflux for several hours. The reaction can also be carried out in the presence of a base such as diisopropylethylamine in a solvent such as an anhydrous dimethylformamide between 0° C. and room temperature for about 2 hours.

In an alternative process a compound of formula I can be made by reacting a compound of formula IV with a compound of formula V:
Y—B(OH)₂ (IV)
L¹-J-L-Z (V)
wherein J, L, L¹, Y and Z are as defined above. The reaction can be carried out under conditions suitable for a Suzuki Coupling Reaction (for review, see for instance A. Suzuki, Pure Appl. Chem., 1991, 63, 419-422), for example, in the presence of a palladium catalyst such as tetrakis(triphenylphosphine)palladium(0), tris(dibenzylideneacetone)dipalladium(0), (1,1′-bis(diphenylphosphino)ferrocene)dichloropalladium or dichloro-(1,4-bis(diphenylphosphino)butane)palladium, in a suitable solvent such as an ether, for example, dimethoxyethane or dioxane or an aromatic hydrocarbon, for example toluene, at an elevated temperature and in the presence of a base such as sodium carbonate or potassium phosphate.

The B(OH₂) moiety can be replaced by, for example, a 4,4,5,5-tetramethyl-1,3,2-dioxaborolan-2-yl moiety. This group can be introduced by converting a methoxy group to a hydroxy group by refluxing with an acid catalyst such as aqueous hydrogen bromide for about five days, or boron tribromide in a solvent such as dichloromethane rising from 0° C. to reflux and reacting for several hours. The hydroxy substituent is then reacted successively with trifluoromethanesulfonic acid anhydride in the presence of a base such as pyridine and a solvent such as dichloromethane at about room temperature for several hours; and then with bis(pinacolato)diboron and a base such as potassium acetate in a solvent such as 1,4-dioxane and a coupling agent such as Pd(dppf)Cl₂ at about 80° C. for several hours.

The B(OH)₂ moiety can also be replaced by, for example, tributyltin. When L¹ is iodine a Stille coupling can then take place in the presence of lithium chloride, copper(I)iodide and a palladium catalyst. The stannane group can be introduced either by adding a di(trialkyltin) compound to the reaction mixture or prereacting it with a compound of formula YCl in the presence of lithium chloride, copper(I)iodide and a palladium catalyst and a solvent such as 1,4-dioxane at about 100° C. for several hours.

When moiety Y is quinoline it can be made by reacting an aniline derivative with 2,2-dimethyl-1,3-dioxane-4,6-dione (Meldrum's acid) in a solvent such as acetonitrile, followed by trimethyl orthoformate generally at reflux for about three hours; or with an appropriate malonate ester under similar conditions. The product is heated in a high boiling solvent, such as Dowtherm A® for about one hour to obtain a quinolin-4(1H)-one. If a mixture of isomers is obtained these can be separated either before or after aromatising the quinoline which can be done by reacting with phosphorous oxychloride at about 80° C. for about 1 hour. Quinolin-2(IH)-ones can be obtained by reacting with an appropriate acetoacetate derivative generally in a solvent such as toluene at about reflux for about 1 day, followed by addition of an acid such as toluene sulphonic acid and further heating at about reflux for about 1 day.

Compounds of formula II can be made by reacting a compound of formula IV with a compound of formula VI:
L¹-J-L¹ (VI)
wherein J and L¹ are as defined above. The reaction is again a Suzuki Coupling Reaction. If necessary the compound of formula VI can be protected. For example when J is a pyridazine the starting chloropyridazinone can be protected with a tetrahydropyran group by heating with 3,4-dihydro-2H-pyran and an acid catalyst such as p-toluenesulfonic acid monohydrate at reflux for about 60 hours. After the Suzuki coupling the protecting group can be removed and the product chlorinated to produce the resulting compound of formula II using phosphorous oxychloride with heating to about 85° C.

Compounds of formula V can be made by reacting a compound of formula III with a compound of formula VI under conditions as described for the reaction between compounds of formulae II and III above. The compound of formula VI may be protected as described above.

A further process for making compounds of formula I involves reacting a compound of formula VII with a compound of formula VIII:
Y-J-LH (VII)
L²-Z (VIII)
wherein J, L, Y and Z are as defined above and L² is a leaving group such as bromine. The reaction conditions are as described above for the reaction between compounds of formulae II and III.

The compound of formula VII can be made by reacting a compound of formula IV with a compound of formula IX:
L¹-J-LH (IX)
wherein L, L¹ and J are as defined above for a Suzuki or Stille Coupling Reaction.

The LH moiety in the compound of formula IX can be made by reacting a chlorine moiety with aqueous ammonia in a solvent such as butanol generally under pressure at about 90° C. for about 2 1/2 hours.

These compounds can be made by reacting compounds bearing two hydroxy moieties with phosphorous oxychloride generally in a solvent such as anhydrous toluene at about reflux in the presence of a base such as triethylamine for about one hour.

This compound when J is pyrimidine can be made by reacting a compound of formula X with a compound of formula XI: 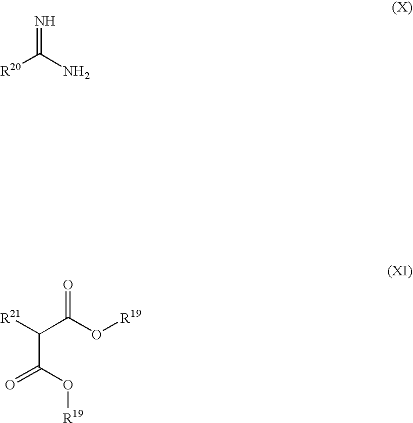
wherein R²⁰ and R²¹ are optional substituents on J as defined above, and R¹⁹ are generally C₁₋₆alkyl groups. The reaction is carried out in a solvent such as ethanol generally for several hours in the presence of a strong base, such as sodium ethoxide. The amidine is usually introduced as the hydrochloride or acetate salt.

Compounds of formula VI where L¹ is chlorine can be made in a variation of this process: when R²⁰ is SH a 6-hydroxy-4-oxo-1,4-dihydropyrimidin-2-thiolate results. The desired thioether can be made by reacting with the appropriate alkyliodide in a solvent such as DMF at about room temperature for several hours. This compound can be converted to a compound of formula VI in which L¹ is chlorine by reacting with POCl₃ in an aprotic solvent such as diethylisopropylamide at about 100° C. for several hours.

Compounds of formula X can be made by bubbling ammonia gas through a solution containing a compound of formula XIII: 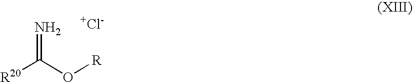
where R is an alkyl group and R²⁰ is as defined above, and the solvent is ROH at about −15° C.

The compound of formula XIII can be made by reacting a compound of formula XIV:
R²⁰—CN (XIV)
wherein R²⁰ is as defined above with an alcohol of formula ROH where R is as defined above in an aprotic solvent such as diethyl ether or the ether ROR and hydrogen chloride gas at about −15° C. for about one hour.

A yet further process for making compounds of formula I in which Y is quinoline involves cyclizing a compound of formula XII: 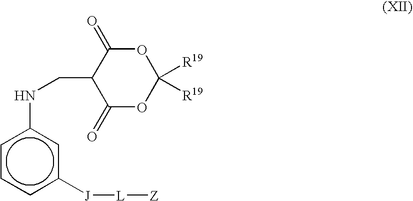
wherein J, L, Z and R¹⁹ are as defined above by heating to reflux in a heat transfer fluid such as Dowtherm A® for about one hour.

Compounds of formula I can be converted to other compounds of formula I by standard methods. For example, nitro groups can be converted to amino groups using a reducing agent such as 10% palladium on carbon under a hydrogen atmosphere generally in the presence of solvents such as methanol and dichloromethane for about two hours. Indeed such reactions can be carried out on any of the starting materials to introduce desired substituents. For example a methoxy group can be introduced on moiety J by displacing a chlorine using sodium methoxide and methanol at reflux for about two hours. A chlorine moiety can be removed by a reducing agent such as 10% palladium on carbon under a hydrogen atmosphere in the presence of a base such as triethylamine and a solvent such as methanol for several hours.

A carboxy group can be converted to an amino group by reacting with diphenylphosphoryl azide in the presence of a base such as triethylamine in a solvent such as toluene. A Curtius rearrangement of the resulting azide, by heating in a solvent such as toluene at reflux for about one hour, following by reacting with 2-methyl-2-propanol for about five hours in a solvent such as toluene and then deprotecting with an acid such as trifluoroacetic acid in a solvent such as dichloromethane yields the desired amine.

A hydroxymethyl group can be converted to a morpholine derivative by reacting with an equivalent of methylsulfoxychloride in the presence of a base such as pyridine in a solvent such as dichloromethane for several hours at about room temperature, and then repeating the process at reflux. The resulting methylsulfoxy compound can then be reacted with the appropriate morpholine in a solvent such as DMF at about 90° C. for several hours to yield the desired morpholine derivative. An analogous procedure can be used to introduce 5- or 6-membered heterocyclic groups, generally using a solvent such as ethanol at a temperature of about 80° C. for about 18 hours.

Bromine groups can be introduced by reacting with a brominating agent such as N-bromosuccinimide in an aprotic solvent such as tetrachloromethane at reflux for about 90 minutes.

A thioether can be converted to the sulfonyl analogue by reacting with Oxone® in a solvent such as methanol at about room temperature for several hours and then at reflux for about two hours. This compound can be converted to the cyanide analogue by reacting with a compound such as sodium cyanide in a solvent such as DMSO for about three days at about room temperature. The sulfonyl compound can be converted to the morpholine or pyrrolidine analogue by reacting with the appropriate morpholine or pyrrolidine in a solvent such as 1,4-dioxane at about 180° C. for about 20 minutes in a microwave reactor. The sulfonyl compound can be converted to a haloalkoxy, alkoxy, cycloalkoxy or heterocyclylalkoxy analogue by reacting with the appropriate haloalcohol generally in a solvent such as tetrahydrofuran in the presence of a strong base such as sodium hydroxide at about 120° C. for about six hours.

Ethers can be converted to alcohols by reacting with hydrochloric acid in a protic solvent such as water for about 2 days at about reflux.

A chlorine atom can be replaced by a haloalkyl group by reacting with the appropriate haloalkylamine in a solvent such as 1,4-dixane at about 160° C. for about 20 minutes in a microwave oven.

A cyano group can be derivatised to a tetrazolyl group by reacting with an azide such as sodium azide generally in the presence of ammonium chloride in a solvent such as DMF at about 120° C. for about two hours.

Where the synthesis of intermediates and starting materials is not described these compounds are commercially available or can be made from commercially available compounds by standard methods. Examples of appropriate methods can be found in the Descriptions.

During any of the above synthetic sequences it may be necessary and/or desirable to protect sensitive or reactive groups on any of the molecules concerned. This may be achieved by means of conventional protecting groups, such as those described in Protective Groups in Organic Chemistry, ed. J. F. W. McOmie, Plenum Press, 1973; and T. W. Greene and P. G. M. Wuts, Protective Groups in Organic Synthesis, John Wiley & Sons, 1991. The protecting groups may be removed at a convenient subsequent stage using methods known from the art.

The following Examples serve to illustrate the preparation of compounds of the present invention.

### Description 1 2-Chloro-4-(quinolin-8-yl)pyrimidine

Pd(PPh₃)₄ (334 mg, 0.29 mmol) was added to a mixture of 2,4-dichloropyrimidine (861 mg, 5.78 mmol), quinoline-8-boronic acid (1.0 g, 5.78 mmol), and 2M aqueous sodium carbonate (2.89 ml, 5.78 mmol) in a mixture of toluene (50 ml) and ethanol (10 ml). The mixture was degassed three times and heated at reflux overnight. The reaction mixture was cooled and diluted with EtOAc (50 ml), washed with water (2×100 ml), sat NaCl (100 ml), dried over Na₂SO₄, filtered and evaporated. The residue was purified by column chromatography on silica: (eluent 2% MeOH in DCM+0.5% NH₄OH) to give the title compound as a white solid (650 mg, 46%). ¹H NMR (500 MHz, CDCl₃) 7.49 (1 H, dd, J 8.3 and 4.2), 7.69 (1 H, t, J 7.9 and 7.6), 7.98 (1 H, d, J 8.1), 8.25 (1 H, dd, J 8.3 and 1.5), 8.43 (1 H, d, J 7.1), 8.46 (1 H, d, J 5.2), 8.69 (1 H, d, J 5.2), 8.97 (1 H, dd, J 3.9 and 1.5).

### Description 2 2-Chloro-6-(quinolin-8-yl)pyrazine

Prepared from 2,6-dichloropyrazine according to the procedure of Description 1 to give a white solid (1.2 g, 86%). ¹H NMR (500 MHz, CDCl₃) 7.48 (1 H, dd, J 8.3 and 4.2), 7.69 (1 H, t, J 8.1 and 7.4), 7.95 (1 H, dd, J 8.1 and 1.2), 8.24 (1 H, dd, J 8.3 and 1.7), 8.27 (1 H, dd, J 7.1 and 1.5), 8.57 (1 H, s), 8.97 (1 H, dd, J 4.2 and 2.0), 9.52 (1 H, s).

### Description 3 5-Chloro-2-(tetrahydropyran-2-yl)pyridazin-3(2H)-one

To a mixture of 5-chloropyridazin-3(2H)one (ES-A-454136) (85 g, 651 mmol), p-toluenesulfonic acid monohydrate (6.91 g, 32 mmol), and 3,4-dihydro-2H-pyran (109 g, 1.302 mol) in tetrahydrofuran (800 ml) was heated at reflux for 60 hours. The cooled mixture was evaporated and the residue dissolved in ethyl acetate (800 ml). The ethyl acetate solution was washed with sat Na₂CO₃, sat. NaCl, dried over MgSO₄, filtered and evaporated to half volume. To this mixture was added triethylamine (100 ml) and silica gel (200 g) and the mixture evaporated to dryness. The residue was loaded onto a silica gel column which had been pre-treated with EtOAc/iso-hexane/triethylamine (80:20:10) and the column eluted with a gradient rising from 5% EtOAc in iso-hexanes to 20% EtOAc in iso-hexanes. The product obtained was triturated with iso-hexanes filtered and dried to give the title compound as an orange solid (88 g, 63%). ¹H NMR (500 MHz, CDCl₃) 1.53-1.62 (1 H, m), 1.66-1.76 (3 H, m), 1.98-2.27 (2 H, m), 3.71-3.76 (1 H, m), 4.11-4.15 (1 H, m), 6.00(1 H, dd, J 10.7 and 2.2), 6.96(1 H, d, J 2.4), 7.79 (1 H, d, J 2.4).

### Description 4 5-(Quinolin-8-yl)-2-(tetrahydropyran-2-yl)pyridazin-3(2H)-one

Prepared from 5-chloro-2-(tetrahydropyran-2-yl)pyridazin-3(2H)-one according to the procedure of Description 1 to give a white solid (1.4 g, 79%). ¹H NMR (500 Hz, CDCl₃) 1.55-1.62 (1 H, m), 1.70-1.86 (3 H, m), 2.05-2.10 (1 H, m), 2.23-2.32 (1 H, m), 3.77-3.84 (1 H, m), 4.19 (1 H, m), 6.18 (1 H, dd, J 10.6 and 2.0), 7.18 (1 H, d, J 2.2), 7.49 (1 H, dd, J 8.3 and 4.2), 7.64 (1 H, dd, J 8.1 and 7.2), 7.75 (1 H, dd, J 7.1 and 1.4), 7.95 (1 H, dd, J 8.2 and 1.3), 8.23 (1 H, dd, J 8.3 and 1.7), 8.35 (1 H, d, J 2.2), 8.94 (1 H, s).

### Description 5 3-Chloro-5-(quinolin-8-yl)pyridazine

A mixture of Description 4 (1.40 g, 4.56 mmol) and phosphorous oxychloride (21.25 ml, 228 mmol) was warmed to 85° C. then allowed to cool to room temperature. The excess phosphorous oxychloride was removed by evaporation and the residue partitioned between dichloromethane and sat NaHCO₃. The organic layer was washed with sat NaCl, dried over Na₂SO₄, filtered and evaporated. The residue was purified by column chromatography on silica: (eluent 2% MeOH in DCM+0.5% NH₄OH) to give the title compound as a white solid (800 mg, 72%). ¹H NMR (360 MHz, CDCl₃) 7.53 (1 H, dd, J 8.3 and 4.1), 7.69 (1 H, t, J 7.8 and 7.5), 7.83 (1 H, dd, J 7.2 and 1.1), 7.97-8.01 (2 H, m), 8.27 (1 H, dd, J 8.3 and 1.5), 8.97 (1 H, d, J 1.7), 9.52 (1 H, d, J 1.7).

### Description 6 4-Chloro-6-(quinolin-8-yl)pyrimidine

To a mixture of 4,6-dichloropyrimidine (1.72 g, 11.6 mmol), quinoline-8-boronic acid (1.0 g, 5.78 mmol) and tripotassium phosphate (2.46 g, 11.6 mmol) in 1,4-dioxane (50 ml) was added Pd(PPh₃)₄ (334 mg, 0.29 mmol). The mixture was degassed three times and heated at reflux overnight. The reaction mixture was cooled and diluted with EtOAc (50 ml), washed with water (2×100 ml), sat. NaCl (100 ml), dried over Na₂SO₄, filtered and evaporated. The residue was purified by column chromatography on silica: (eluent 2% MeOH in DCM+0.5% NH₄OH) to give the title compound as a white solid (200 mg, 14%). ¹H NMR (500 MHz, CDCl₃) 7.50 (1 H, dd, J 8.1 and 4.2), 7.71 (1 H, t, J 7.8 and 7.6), 7.98 (1 H, dd, J 8.1 and 1.3), 8.26(1 H, dd, J 8.4 and 1.8), 8.41 (1 H, dd, J 7.3 and 1.5), 8.56 (1 H, d, J 1.0), 9.01 (1 H, dd, J 4.2 and 2.0), 9.12 (1 H, d, J 1.0).

### Description 7 2-(Tetrahydro-2H-pyran-2-yl)-5-{[4-trifluoromethylphenyl]amino}pyridazin-3(2H)-one

To a mixture of Description 3 (1.0 g, 4.66 mmol), and (4-trifluoromethyl)aniline (0.59 ml, 4.66 mmol) in anhydrous 1,4-dioxane (50 ml) was added cesium carbonate (2.13 g, 6.52 mmol), 4,5-bis(diphenylphosphino)-9,9-dimethylxanthene (161 mg, 0.28 mmol), and Pd₂(dba)₃ (90 mg, 0.09 mmol). The mixture was degassed three times and heated at reflux overnight. The mixture was cooled and diluted with EtOAc (50 ml), washed with water (2×100 ml), sat. NaCl, (100 ml), dried over Na₂SO₄, filtered and evaporated. The residue was purified by column chromatography on silica: (eluent 2% MeOH in DCM+0.5% NH₄OH) to give the title compound as a white solid (1.1 g, 69%). ¹H NMR (500 MHz, CDCl₃) 1.50-1.60 (1 H, m), 1.60-1.78 (3 H, m), 1.95-2.08 (1 H, m), 2.08-2.22 (1 H, m), 3.69 (1 H, t, J 11.5 and 9.8), 4.08 (1 H, d, J 11.0), 6.01 (1 H, d, J 10.7), 6.30(1 H, d, J 2.6), 7.24 (2 H, d, J 8.4), 7.36 (1 H, s), 7.58 (2 H, d, J 8.4), 7.67 (1 H, d, J 2.6).

### Description 8 6-Chloro-N-[4-trifluoromethylphenyl]pyridazin-4-amine

Prepared from Description 7 according to the procedure of Description 5 to give the title compound as a pale brown solid (250 mg, 28%). ¹H NMR (400 MHz, CDCl₃) 7.10 (1 H, s), 7.37 (2 H, d, J 8.2), 7.68 (2 H, d, J 8.2), 8.92 (1 H, s).

### Description 9 4-Chloro-N-[4-trifluoromethylphenyl]-1,3,5-triazin-2-amine

To a solution of 2,4-dichlorotriazine [WO-A-0125220] (3.0 g, 20 mmol) in anhydrous N,N-dimethylformamide (20 ml) cooled in an ice-bath was added diisopropylethylamine (3.46 ml, 20 mmol) followed by 4-(trifluoromethyl)aniline (2.27 ml, 18.2 mmol) and the resulting mixture stirred at 0° C. for 15 mins, then allowed to warm to room temperature where it was stirred for 2 hours. The mixture was diluted with ethyl acetate (150 ml) and washed with water (3×200 ml), sat. NaCl (100 ml), dried over Na₂SO₄, filtered and evaporated to give the title compound as a white solid (4.7 g, 86%). ¹H NMR (500 MHz, DMSO-d₆) 7.74 (2 H, d, J 8.6), 7.91 (2 H, d, J 8.6), 8.74 (1 H, s), 11.09 (1 H, s).

### Description 10 Quinolin-7-yl trifluoromethanesulfonate

To an ice-bath cooled suspension of 7-hydroxyquinoline (6.23 g, 42.9 mmol), and pyridine (4.51 ml, 55.77 mmol) in anhydrous dichloromethane (100 ml) was added dropwise trifluoromethanesulfonic anhydride (7.94 ml, 47.19 mmol) and the resulting mixture stirred at room temperature overnight. The mixture was washed with water (250 ml), sat. NaCl (150 ml), dried over Na₂SO₄, filtered and evaporated to give the title compound as a beige solid (11.3 g, 95%). ¹H NMR (400 Hz, CDCl₃) 7.47-7.51 (2 H, m), 7.93 (1 H, d, J 9.0), 8.04(1 H, d, J 2.4), 8.22 (1 H, dd, J 8.2 and 0.7), 9.00 (1 H, dd, J 4.3 and 1.9).

### Description 11 7-(4,4,5,5-Tetramethyl-1,3,2-dioxaborolan-2-yl)quinoline

To a mixture of Description 10 (8.7 g, 31.4 mmol), bis(pinacolato)diboron (8.8 g, 34.5 mmol), and potassium acetate (9.25 g, 94.2 mmol) in anhydrous 1,4-dioxane (150 ml) was added Pd(dppf)Cl₂ (860 mg, 0.94 mmol). The mixture de-gassed three times and heated at 80° C. overnight. The mixture was cooled and diluted with ethyl acetate (200 ml), washed with water (required a filtration through celite), sat. NaCl, dried over Na₂SO₄, filtered and evaporated to give the title compound as a brown oil which solidified on standing. ¹H NMR (400 MHz, CDCl₃) 1.39 (12 H, s), 7.42 (1 H, dd, J 8.2 and 3.9), 7.79 (1 H, d, J 7.8), 7.90 (1 H, d, J 9.0), 8.15 (1 H, dd, J 8.6 and 1.2), 8.61 (1 H, s), 8.90 (1 H, dd, J 4.3 and 2.0).

### Description 12 2-Chloro-6-(quinolin-7-yl)pyrazine

Prepared from 2,6-dichloropyrazine and Description 11 according to the procedure of Description 1 to give a white solid (825 mg, 59%). ¹H NMR (500 MHz, CDCl₃) 7.47 (1 H, dd, J 8.3 and 4.2), 7.97 (1 H, d, J 8.6), 8.21 (1 H, d, J 7.8), 8.29 (1 H, dd, J 8.6 and 1.7), 8.58 (1 H, s), 8.74 (1 H, s), 8.99 (1 H, dd, J 4.2 and 1.7), 9.13 (1 H, s).

### Description 13 2-Chloro-4-(quinolin-7-yl)pyrimidine

Prepared from Description 11 according to the procedure of Description 1 to give a pale brown solid (822 mg, 59%). ¹H NMR (500 MHz, CDCl₃) 7.50 (1 H, dd, J 8.3 and 4.2), 7.85 (1 H, d, J 5.2), 7.97 (1 H, d, J 8.6), 8.22 (1 H, d, J 7.9), 8.37 (1 H, dd, J 8.6 and 2.0), 8.57 (1 H, d, J 5.2), 8.77 (1 H, s), 9.00 (1 H, dd, J 4.2 and 1.5).

### Description 14 6-(Quinolin-7-yl)pyrimidin-4-amine

Prepared from 4-amino-6-chloropyrimidine (WO-A-0245652) and Description 11 according to the procedure of Description 1 to give a pale brown solid (600 mg, 38%). ¹H NMR (400 MHz, DMSO-d₆) 7.01 (2 H, br s), 7.12 (1 H, d, J 1.0), 7.59 (1 H, dd, J 8.2 and 4.2), 8.10 (1 H, d, J 8.6), 8.21 (1 H, dd, J 8.6 and 1.6), 8.42 (1 H, dd, J 8.2 and 0.5), 8.53 (1 H, d, J 0.7), 8.62 (1 H, s), 8.98 (1 H, d, J 1.6).

### Description 15 3-Chloro-5-(quinolin-7-yl)pyridazine

Prepared from Description 3 and Description 11 according to the procedures of Descriptions 1 and 5 respectively. ¹H NMR (500 MHz, CDCl₃) 7.53 (1 H, dd, J 8.3 and 4.2), 7.82 (1 H, dd, J 8.6 and 1.7), 7.85 (1 H, d, J 2.0), 8.03 (1 H, d, J 8.4), 8.25 (1 H, d, J 8.1), 8.46(1 H, d, J 1.5), 9.03 (1 H, dd, J 4.2 and 1.5), 9.54 (1 H, d, J 2.0).

### Description 16 4-Amino-6-chloro-5-methoxypyrimidine

A mixture of 4,6-dichloro-5-methoxypyrimidine (5.0 g, 27.9 mmol), 33% aqueous ammonia (30 ml) and 1-butanol (15 ml) was heated at 90° C. in a sealed tube for 2.5 hours. The mixture was allowed to cool and the precipitate removed by filtration, and dried to give the title compound as a white solid (1.8 g, 40%). ¹H NMR (400 MHz, DMSO-d₆) 3.71 (3 H, s), 7.30 (2 H, br s), 7.96 (1 H, s).

### Description 17 5-Methoxy-6-(quinolin-7-yl)-pyrimidin-4-amine

Prepared from Description 16 and Description 11 according to the procedure of Description 1 to give an orange solid (690 mg, 47%). ¹H NMR (400 MHz, CDCl₃) 3.57 (3 H, s), 5.49 (2 H, br s), 7.46 (1 H, dd, J 8.2 and 4.2), 7.93 (1 H, d, J 8.5), 8.21 (1 H, dd, J 8.5 and 1.5), 8.83 (1 H, s), 8.99 (1 H, d, J 1.6).

### Description 18 4-Amino-6-chloro-5-methylpyrimidine

Prepared from 4,6-dichloro-5-methylpyrimidine according to the procedure of Description 16 to give a white solid (3.1 g, 70%). ¹H NMR (400 MHz, DMSO-d₆) 2.08 (3 H, s), 7.11 (2 H, br s), 8.06 (1 H, s).

### Description 19 5-Methyl-6-(quinolin-7-yl)pyrimidin-4-amine

Prepared from Description 18 and Description 11 according to the procedure of Description 1 to give a beige solid (410 mg, 29%). ¹H NMR (400 MHz, CDCl₃) 2.22 (3 H, s), 5.09 (2 H, br s), 7.46 (1 H, dd, J 8.2 and 4.2), 7.79 (1 H, dd, J 8.4 and 1.5), 7.94 (1 H, d, J 8.4), 8.20-8.24 (2 H, m), 8.59 (1 H, s), 8.98 (1 H, d, J 1.5).

### Description 20 4-Amino-6-chloro-5-fluoropyrimidine

Prepared from 4,6-dichloro-5-fluoropyrimidine (DE-A-10014607) according to the procedure of Description 16 to give a white solid (5.8 g, 94%). ¹H NMR (400 MHz, DMSO-d₆) 7.69 (2 H, br s), 8.07 (1 H, s).

### Description 21 5-Fluoro-6-(quinolin-7-yl)pyrimidin-4-amine

Prepared from Description 20 and Description 11 according to the procedure of Description 1 to give a beige solid (900 mg, 64%). ¹H NMR (400 MHz, DMSO-d₆) 7.47 (2 H, br s), 7.63 (1 H, dd, J 8.2 and 4.2), 8.13 (1 H, d, J 8.6), 8.19 (1 H, d, J 8.6), 8.34 (1 H, d, J 2.3), 8.45 (1 H, d, J 8.1), 8.59 (1 H, s), 9.00 (1 H, d, J 1.4).

### Description 22 4-Amino-6-chloro-2-methoxypyrimidine

Sodium methoxide (12 ml of a 25% wt solution in methanol) was added to methanol (300 ml) and to this mixture added 4-amino-2,6-dichloropyrimidine (5.00 g, 30.5 mmol). The resultant solution was heated at reflux for 2 hours and then evaporated to dryness. The residue was treated with water (250 ml) and the precipitate which formed filtered and dried in vacuo to give the title product as a white solid (3 g, 61%). ¹H NMR (400 MHz, CDCl₃) 3.92 (3 H, s), 5.21 (2 H, br s), 6.14 (1 H, s).

### Description 23 2-Methoxy-6-(quinolin-7-yl)pyrimidin-4-amine

Prepared from Description 22 and Description 11 according to the procedure of Description 1 to give an orange solid (1.15 mg, 65%). ¹H NMR (400 MHz, DMSO-d₆) 3.93 (3 H, s), 6.83 (1 H, s), 7.05 (2 H, br s), 7.59 (1 H, dd, J 8.3 and 4.2), 8.09 (1 H, d, J 8.6), 8.20 (1 H, dd, J 8.6 and 1.7), 8.42 (1 H, d, J 8.3), 8.63 (1 H, s), 8.98 (1 H, d, J 1.6).

### Description 24 4-Amino-6-chloro-2-methylpyrimidine

Prepared from 4,6-dichloro-2-methylpyrimidine according to the procedure of Description 16 to give a pale yellow solid (3.5 g, 46%). ¹H NMR (400 MHz, DMSO-d₆) 2.29 (3 H, s), 7.16 (2 H, br s), 7.38 (1 H, s).

### Description 25 2-Methyl-6-(quinolin-7-yl)pyrimidin-4-amine

Prepared from Description 24 and Description 11 according to the procedure of Description 1 to give an orange solid (980 mg, 59%). ¹H NMR (400 MHz, CDCl₃) 2.63 (3 H, s), 5.07 (2 H, br s), 6.82 (1 H, s), 7.43 (1 H, dd, J 8.3 and 4.2), 7.90 (1 H, d, J 8.6), 8.18 (1 H, d, J 7.7), 8.25 (1 H, dd, J 8.6 and 1.7), 8.63 (1 H, s), 8.96 (1 H, d, J 1.7).

### Description 26 7-Methoxy-3-methylquinoline

To a nitrogen flushed suspension of 2-chloro-7-methoxy-3-methylquinoline [Organic Preparations and Procedures International (1990), 22(5), 579-88] (7.20 g, 34.7 mmol) and triethylamine (5.32 ml, 38.17 mmol) in methanol was added a spatula end of 10% Palladium on carbon and the resulting mixture stirred under a balloon of hydrogen overnight. The catalyst was removed by filtration and the filtrate evaporated. The residue was dissolved in dichloromethane (100 ml) and washed with water (150 ml), dried over Na₂SO₄, filtered and evaporated to give the title compound as a pale brown oil (6 g, 99%). ¹H NMR (400 MHz, CDCl₃) 2.47 (3 H, s), 3.94 (3 H, s), 7.16 (1 H, dd, J 8.9 and 2.5), 7.39 (1 H, d, J 2.5), 7.61 (1 H, d, J 8.9), 7.82 (1 H, t, J 0.9), 8.68 (1 H, d, J 2.2).

### Description 27 3-Methylquinolin-7-ol

A mixture of Description 26 (6.0 g, 34.6 mmol) and 48% aqueous HBr (150 ml) was heated at reflux for 5 days. The mixture was cooled and basified by the careful addition of 33% aqueous ammonia. The resulting precipitate was removed by filtration, washed with water, and dried in-vacuo to give the title compound as a pink solid (4.6 g, 84%). ¹H NMR (400 MHz, DMSO-d₆) 2.41 (3 H, s), 7.13 (1 H, dd, J 8.8 and 2.4), 7.22 (1 H, d, J 2.4), 7.71 (1 H, d, J 8.8), 7.96 (1 H, t, J 0.7), 8.61 (1 H, d, J 2.2), 10.01 (1 H, s).

### Description 28 6-(3-Methylquinolin-7-yl)pyrimidin-4-amine

Prepared from Description 27 according to the procedures of Descriptions 10, 11 and 14 respectively to give a light brown solid (950 mg, 57%). ¹H NMR (400 MHz, DMSO-d₆) 2.51 (3 H, s), 6.99 (2 H, br s), 7.10 (1 H, s), 7.99 (1 H, d, J 8.6), 8.16 (2 H, m), 8.52 (1 H, s), 8.57 (1 H, s), 8.62 (1 H, s), 8.82 (1 H, s).

### Description 29 6-Chloro-5-nitro-N-[4-(trifluoromethyl)phenyl]pyrimidin-4-amine

To a suspension of 4,6-dichloro-5-nitropyrimidine (5.00 g, 25.8 mmol) in anhydrous tetrahydrofuran (100 ml) was added sodium hydrogen carbonate (2.38 g, 28.38 mmol) and 4-(trifluoromethyl)aniline (3.24 ml, 25.8 mmol), and the resulting mixture stirred at room temperature for 3 days. The mixture was filtered and the filtrate evaporated. The residue triturated with diethyl ether and filtrate from trituration evaporated to give the title compound as a yellow solid (3.2 g, 38%). ¹H NMR (400 MHz, CDCl₃) 7.70 (4 H, q, J 9.0), 8.55 (1 H, s), 8.92 (1 H, s), 9.25 (1 H, br s).

### Description 30 4-Chloro-6-(quinolin-5-yl)pyrimidine

Prepared from 4,6-dichloropyrimidine and quinoline-5-boronic acid according to the procedure of Description 1 to give a white solid. ¹H NMR (400 MHz, CDCl₃) 7.49 (1 H, dd, J 8.6 and 4.2), 7.69 (1 H, d, J 1.1), 7.76 (1 H, dd, J 7.2 and 1.3), 7.83 (1 H, dd, J 8.3 and 7.2), 8.28 (1 H, dd, J 7.4 and 1.0), 8.65 (1 H, dd, J 7.2 and 0.8), 8.65 (1 H, dd, J 4.2 and 1.9), 9.17 (1 H, d, J 1.0).

### Description 31 6-(Quinolin-6-yl)pyrimidin-4-amine

Prepared from 4-amino-6-chloropyrimidine (WO-A-0245652) and quinoline-6-boronic acid according to the procedure of Description 1 to give a pale brown solid. ¹H NMR (400 MHz, DMSO-d₆) 7.00 (2 H, br s), 7.06(1 H, d, J 1.2), 7.59(1 H, dd, J 8.6 and 4.3), 8.11 (H, d, J 9.0), 8.32 (1 H, d, J 9.0 and 2.0), 8.50-8.53 (2 H, m), 8.66 (1 H, d, J 2.0), 8.95 (1 H, dd, J 4.3 and 1.6).

### Description 32 3-Methylquinolin-7-ol

To a solution of 7-methoxy-2-methylquinoline (J. Med. Chem (1998), 41(21), 4062-4079) (5 g, 29 mmol) in anhydrous dichloromethane (20 ml) was added boron tribromide (58 ml of a 1M solution in CH₂Cl₂, 58 mmol) at 3° C. After stirring for 30 mins the reaction mixture was allowed to warm to room temperature and stirred for a further 2 hours. The reaction mixture was then refluxed for 16 hours. The cooled reaction mixture was adjusted to pH 7 with sat. sodium hydrogen carbonate solution, extracted with dichloromethane (2×50 ml), dried over MgSO₄, filtered and evaporated. The residue was purified by column chromatography on silica: (eluent 2% MeOH in DCM) to give the title compound (1.5 g, 32%). ¹H NMR (400 MHz, CDCl₃) 2.65 (3 H, s), 6.84 (1 H, dd, J 2.5, 8.8), 7.06 (1 H, d, J 8.2), 7.22 (1 H, d, J 2.3), 7.40 (1 H, d, J 9.0), 7.87 (1 H, d, J 8.6).

### Description 33 6-(2-Methylquinolin-7-yl)pyrimidin-4-amine

Prepared from Description 32 according to the procedures of Descriptions 10, 11, and 14 respectively. ¹H NMR (400 MHz, DMSO-d₆) 2.68 (3 H, s), 6.97 (2 H, br s), 7.07 (1 H, d, J 1.2), 7.46 (1 H, d, J 8.2), 8.02 (1 H, d, J 8.6), 8.11 (1 H, dd, J 8.2 and 1.6), 8.28 (1 H, d, J 8.6), 8.49 (1 H, d, J 2.0), 8.51 (1 H, d, J 1.2).

### Description 34 5-{[(4-Fluoro-3-methoxyphenyl)amino]methylene )-2,2-dimethyl-1,3-dioxane-4,6-dione

To a stirred solution of 4-fluoro-3-methoxyaniline (20 g, 142 mmol) in acetonitrile (200 ml) was added 2,2-dimethyl-1,3-dioxane-4,6-dione (Meldrum's acid) (22.5 g, 156 mmol) followed by trimethyl orthoformate (18.6 ml, 170 mmol). The mixture was heated to reflux for 3 hours. The cooled mixture was filtered to give the title compound (30.9 g, 74%). ¹H NMR (400 MHz, CDCl₃) 1.76 (6 H, s), 3.94 (3 H, s), 6.76-6.83(2 H, m), 7.14 (1 H, dd, J 10.5 and 8.4), 8.56 (1 H, d, J 14.4), 11.23 (1 H, d, J 14.4).

### Description 35 6-Fluoro-7-methoxyquinolin-4(1H)-one

To a boiling solution of Dowtherm A® (80 ml) was added in portions Description 34 (30.9 g, 105 mmol). Heating was continued for 1 hour after the addition was complete and then the mixture cooled to room temperature. The mixture was poured into hexane (200 ml) and filtered. The filtrate was washed with more hexane to give a mixture of the title compound and 6-fluoro-5-methoxyquinolin-4(1H)-one in a 2:1 ratio (22.6 g, 100%).

### Description 36 4-Chloro-6-fluoro-7-methoxyquinoline

A suspension of the crude product of Description 35 (22.6 g, 117 mmol) in phosphorous oxychloride (110 ml, 1.18 mol) was heated at 80° C. for 1 hour. The reaction mixture was allowed to cool and evaporated. The residue was neutralised with saturated sodium bicarbonate solution, extracted with DCM (3×200 ml) and evaporated. The residue was purified by column chromatography on silica: (eluent 2% MeOH in DCM) to give the title compound (11 g, 44%). ¹H NMR (400 MHz, CDCl₃) 4.05 (3 H, s), 7.40 (1 H, dd, J 4.7 and 0.8), 7.53 (1 H, d, J 8.2), 7.85 (1 H, d, J 11.7), 8.68 (1 H, dd, J 4.7 and 0.8).

### Description 37 6-Fluoro-7-methoxyquinoline

Prepared from Description 36 according to the procedure of Description 26. ¹H NMR (400 MHz, CDCl₃) 4.04 (3 H, s), 7.29-7.33 (1 H, m), 7.43 (1 H, d, J 11.3), 7.52 (1 H, d, J 8.2), 8.05 (1 H, dd, J 8.2 and 1.6), 8.82 (1 H, dd, J 4.3 and 1.2).

### Description 38 6-Fluoroquinolin-7-ol

Prepared from Description 37 according to the procedure of Description 27 to give an off white solid (5.1 g, 60%). ¹H NMR (400 MHz, DMSO-d₆) 7.37-7.41 (1 H, m), 7.44 (1 H, d, J 8.4), 7.78 (1 H, d, J 11.9), 8.29 (1 H, dd, J 8.2 and 1.4), 8.78 (1 H, dd, J 4.4 and 1.4), 10.80-11.04 (1 H, br s).

### Description 39 6-(6-Fluoroquinolin-7-yl)pyrimidin-4-amine

Prepared from Description 38 according to the procedures of Descriptions 10, 11 and 14 respectively. ¹H NMR (400 MHz, DMSO-d₆) 7.01 (1 H, s), 7.10 (2 H, s), 7.63 (1 H, m), 7.94 (1 H, d, J 12.1), 8.40 (1 H, d, J 8.2), 8.53 (1 H, d, J 1.2), 8.63 (1 H, d, J 7.4), 8.96 (1 H, dd, J 4.1 and 1.8).

### Description 40 2-Fluoro-3-methoxyaniline

To a solution of 2-fluoro-3-methoxy benzoic acid [Synlett (1991), (10), 731-2] (15.0 g, 88 mmol) and triethylamine (13.49 ml, 96.8 mmol) in toluene (300 ml) was added diphenylphosphoryl azide (20.9 ml, 96.8 mmol) and the resulting mixture heated at reflux for 1 hour. After this time 2-methyl-2-propanol (12.5 ml, 132 mmol) was added and heating continued for 5 hours. The mixture was cooled and evaporated, and the residue partitioned between water and dichloromethane. The dichloromethane layer was dried over Na₂SO₄, filtered through a 1 inch plug of silica and evaporated. The residue was dissolved in dichloromethane (200 ml) and trifluoroacetic acid (25 ml) added, and the resulting mixture stirred at room temperature overnight. The mixture was evaporated and the residue partitioned between dichloromethane and sat. K₂CO₃, the dichloromethane layer was dried over Na₂SO₄, filtered and evaporated. The residue was purified by column chromatography on silica: (eluent with 15% EtOAc in isohexanes) to give the title compound as a pale yellow oil (10.8 g, 87%). ¹H NMR (400 MHz, CDCl₃) 3.72 (2 H, br s), 3.85 (3 H, s), 6.34-6.41 (2 H, m), 6.81-6.86 (1 H, m).

### Description 41 6-(8-Fluoroquinolin-7-yl)pyrimidin-4-amine

Prepared from Description 40 according to the procedures of Descriptions 34, 35, 36, 26, 27, 10, 11 and 14 respectively. ¹H NMR (400 MHz, MeOD) 7.14 (1 H, t, J 1.6), 7.49 (1 H, dd, J 6.7 and 3.2), 7.58 (1 H, d, J 7.6), 7.69 (1 H, dd, J 8.2 and 4.3), 7.87 (1 H, dd, J 8.7 and 0.9), 8.07 (1 H, dd, J 8.5 and 6.7), 8.48 (1 H, dd, J 8.5 and 1.8), 8.51 (1 H, d, J 1.2), 8.96(1 H, dd, J4.3 and 1.5).

### Description 42 6-(6-Trifluoromethylquinoline-7-yl)pyrimidin-4-amine

Prepared from 4-trifluoromethyl-3-methoxyaniline according to the procedures of Descriptions 34, 35, 36, 26, 27, 10, 11 and 14 respectively to give a brown solid. ¹H NMR (400 MHz, CDCl₃) 5.21 (2 H, br s), 6.63 (1 H, s), 7.55 (1 H, dd, J 8.3 and 4.2), 8.19 (1 H, s), 8.29 (2 H, m), 8.68 (1 H, s), 9.08 (1 H, dd, J4.2 and 1.5).

### Description 43 8-Methyl-7-(4,4,5,5-tetramethyl-1,3,2-dioxaborolan-2-yl)quinoline

Prepared from 3-methoxy-2-methylaniline according to the procedures of Descriptions 34, 35, 36, 26, 27, 10, and 11 respectively to give an orange oil. ¹H NMR (400 MHz, CDCl₃) 1.40 (12 H, s), 3.08 (3 H, s), 7.40 (1 H, dd, J 8.2 and 3.9), 7.62 (1 H, d, J 8.2), 7.85 (1 H, d, J 8.2), 8.10 (1 H, dd, J 8.2 and 2.0), 8.96 (1 H, dd, J 4.3 and 2.0).

### Description 44 6-(8-Methylquinolin-7-yl)pyrimidin-4-amine

Prepared from Description 43 according to the procedure of Description 14 to give a pale brown solid. ¹H NMR (400 MHz, CDCl₃) 2.85 (3 H, s), 5.12 (2 H, br s), 6.59 (1 H, d, J 1.2), 7.44 (1 H, dd, J 8.3 and 4.2), 7.58 (1 H, d, J 8.5), 7.73 (1 H, d, J 8.5), 8.16 (1 H, dd, J 8.2 and 1.8), 8.73 (1 H, d, J 1.0), 9.00 (1 H, dd, J 3.9 and 1.6).

### Description 45 5-Fluoro-6-(8-methylquinolin-7-el)pyrimidin-4-amine

Prepared from Description 20 and Description 43 according to the procedure of Description 1 to give a white solid (300 mg, 26%). ¹H NMR (400 MHz, CDCl₃) 2.77 (3 H, d, J 2.2), 5.26 (2 H, br s), 7.47 (1 H, dd, J 8.2 and 4.3), 7.56 (1 H, d, J 8.6), 7.77 (1 H, d, J 8.6), 8.18 (1 H, dd, J 8.2 and 2.0), 8.50 (1 H, d, J 2.4), 9.01 (1 H, dd, J 4.3 and 2.0).

### Description 46 6-(Isoquinoline-7-yl)pyrimidin-4-amine

Prepared from 7-methoxyisoquinoline according to the procedures of Descriptions 27, 10, 11, and 14 respectively to give a white solid. ¹H NMR (500 MHz, DMSO-d₆) 7.04 (2 H, br s), 7.08 (1 H, d, J 1.1), 7.88 (1 H, d, J 5.7), 8.08 (1 H, d, J 8.7), 8.35 (1 H, dd, J 8.7 and 1.7), 8.52 (1 H, d, J 0.9), 8.56 (1 H, d, J 5.7), 8.80 (1 H, s), 9.47 (1 H, s).

### Description 47 6-(8-Fluoroquinolin-7-yl)-5-methylpyrimidin-4-amine

Prepared from Description 40 according to the procedures of Descriptions 34, 35, 36, 26, 27, 10, 11 and 19 respectively. ¹H NMR (400 MHz, CDCl₃) 2.10 (3 H, d, J 3.3), 5.02 (2 H, s), 7.56-7.52 (1 H, m) 8.26-8.24 (1 H, m), 8.60(1 H, s), 9.03 (1 H, dd, J 4.2 and 1.6).

### Description 48 5-Methoxy-2-methylpyrimidine-4,6-diol

Sodium (7.00 g, 305.25 mmol), cut into small chunks, was added portionwise to anhydrous ethanol (300 ml). Once all the sodium had dissolved the mixture was cooled in an ice-bath and acetamidine hydrochloride (9.57 g, 101.75 mmol) was added and the mixture stirred for 20 mins. To this mixture was added dropwise a solution of methoxy dimethylmalonate (15.0 g, 92.5 mmol) in ethanol (50 ml), and once addition was complete the mixture was stirred at room temperature overnight. The ethanol was removed by evaporation and the residue dissolved in water. The mixture was acidified by addition of conc. HCl and the resulting precipitate were removed by filtration and dried in vacuo to give the title compound (8 g, 55%) as a white solid. ¹H NMR (400 MHz, DMSO-d₆) 2.19 (3 H, s), 3.59 (3 H, s), 11.70 (2 H, brs).

### Description 49 4,6-Dichloro-5-methoxy-2-methylpyrimidine

To a suspension of Description 48 (7.99 g, 51.2 mmol) and triethylamine (7.14 ml, 51.2 mmol) in anhydrous toluene (100 ml) heated at 100° C. was added dropw ise a solution of phosphorous oxychloride (10.5 ml, 112.6 mmol) in toluene (50 ml). After complete addition the mixture was heated at reflux for 1 hour. The mixture was cooled in an ice bath and quenched by the careful addition of cold water (100 ml). The organic layer was washed with sat. NaHCO₃, sat. NaCl, and evaporated to dryness to give the title compound (9.5 g, 96%). ¹H NMR (400 MHz, CDCl₃) 2.65 (3 H, s), 3.95 (3 H, s).

### Description 50 6-Chloro-5-methoxy-2-methylpyrimidin-4-amine

Prepared from Description 49 according to the procedure of Description 16 to give a white solid (6.7 g, 78%). ¹H NMR (400 MHz, DMSO-d₆) 2.26 (3 H, s), 3.67 (3 H, s), 7.15 (2 H, br s).

Descriptions 51-53 were prepared from the indicated Description compounds following the procedure of Description 1.

### Description 51 5-Methoxy-2-methyl-6-quinoline-7-ylpyrimidin-4-amine

Description 50 and Description 11 gave a beige solid (775 mg, 50%). ¹H NMR (400 MHz, CDCl₃) 2.57 (3 H, s), 3.54 (3 H, s), 5.17 (2 H, s), 7.45 (1 H, dd, J 8.3 and 4.1), 7.91 (1 H, d, J 8.6), 8.20 (2 H, dd, J 8.4 and 1.6), 8.80 (1 H, s), 8.97 (1 H, dd, J 4.2 and 1.8).

### Description 52 2-Methyl-6-(8-methylquinolin-7-yl)pyrimidin-4-amine

Description 24 and Description 43 gave a beige solid (420 mg, 45%). ¹H NMR (500 MHz, CDCl₃) 2.62 (3 H, s), 2.82 (3 H, s), 5.04 (2 H, br s), 7.42 (1 H, dd, J 8.2 and 4.2), 7.56 (1 H, d, J 8.4), 7.72 (1 H, d, J 8.4), 8.14 (1 H, dd, J 8.3 and 1.8), 8.99 (1 H, dd, J 4.2 and 1.8).

### Description 53 5-Methyl-6-(8-methylquinolin-7-yl)pyrimidin-4-amine

Description 18 and Description 43 gave a beige solid (530 mg, 57%). ¹H NMR (400 MHz, CDCl₃) 1.92 (3 H, s), 2.63 (3 H, s), 4.98 (2 H, br s), 7.39 (1 H, d, J 8.4), 7.45 (1 H, dd, J 8.3 and 4.2), 7.75 (1 H, d, J 8.4), 8.17 (1 H, dd, J 8.2 and 1.8), 8.58(1 H, s), 9.00 (1 H, dd, J4.2 and 1.8).

### Description 54 4-Fluoro-6-methoxy-5-trifluoromethylpyrimidine

To a rapidly stirred mixture of 1-methoxy(perfluoro-2-methylprop-1-ene) (25.0 g, 118 mmol), and formaridine acetate (18.4 g, 177 mmol) in a mixture of water (120 ml) and dichloromethane (120 ml) cooled in an ice bath was added dropwise a solution of sodium hydroxide (18.88 g, 472 mmol) in water (100 ml). After complete addition the mixture was stirred for 30 mins. Then the dichloromethane layer was separated, washed with 1N HCl (150 ml), water (150 ml), dried over Na₂SO₄, filtered and evaporated to give the title compound (8 g, 34%) as a clear oil. ¹H NMR (500 MHz, CDCl₃) 4.15 (3 H, s), 8.60 (1 H, s).

### Description 55 6-Methoxy-5-trifluoromethylpyrimidin-4-amine

A mixture of Description 54 (8 g, 40.8 mmol) in butan-1-ol and ammonium hydroxide (50 ml) was heated in a large (200 ml capacity) sealed tube at 90° C. for 1 hour. The mixture was cooled and the resulting precipitate removed by filtration, and dried to give the title compound (3.3 g, 42%) as a white crystalline solid. ¹H NMR (500 MHz, DMSO-d₆) 3.91 (3 H, s), 7.20 (2 H, br s), 8.23 (1 H, s).

### Description 56 6-Chloro-5-trifluoromethylpyrimidin-4-amine

A mixture of Description 55 (800 mg, 4.14 mmol) and phosphorous oxychloride (7.7 ml, 82.8 mmol) were heated at 100° C. for 4 days. Excess phosphorous oxychloride was removed by rotary evaporation and the residue was taken up into dichloromethane and carefully basified by the addition of sat. NaHCO₃.

The organic layer was separated and dried over Na₂SO₄, filtered and evaporated to give the title compound as a 4:1 mix of product and starting material (450 mg, 55%). ¹H NMR (500 MHz, CDCl₃) 5.77 (2 H, br s), 8.39 (1 H, s).

### Description 57 6-Quinolin-7-yl-5-trifluoromethylpyrimidin-4-amine

Prepared from Description 56 and Description 11 according to the procedure of Description 1 to give a beige solid (100 mg, 15%). ¹H NMR (400 MHz, DMSO-d₆) 7.61 (1 H, dd, J 8.2 and 4.3), 7.64 (1 H, d, J 8.3), 8.00 (1 H, s), 8.06 (1 H, d, J 8.3), 8.44 (1 H, d, J 8.1), 8.58 (1 H, s), 8.98 (1 H, dd, J 4.3 and 2.0), 10.13 (1 H, br s).

### Description 58 6-Chloro-5-ethylpyrimidin-4-amine

Prepared from ethyl diethylmalonate and formamidine acetate according to the procedures of Descriptions 48, 49, and 16 respectively. ¹H NMR (400 MHz, DMSO-d₆) 1.05 (3 H, t, J 7.4), 2.57 (2 H, q, J 7.4), 7.12 (2 H, br s), 8.06 (1 H, s).

### Description 59 5-Ethyl-6-quinolin-7-ylpyrimidin-4-amine

Prepared from Description 58 and Description 11 according to the procedure of Description 1 to give a white solid (1.05 g, 84%). ¹H NMR (400 MHz, CDCl₃) 1.06 (3 H, t, J 7.3), 2.51 (2 H, q, J 7.3), 6.88 (2 H, br d), 7.60 (1 H, dd, J 8.3 and 4.2), 7.67 (1 H, dd, J 8.4 and 1.2), 8.04 (1 H, s), 8.07 (1 H, d, J 8.4), 8.34 (1 H, s), 8.43 (1 H, d, J 8.2), 8.96 (1 H, dd, J 4.2 and 1.2).

### Description 60 6-Chloro-2-cyclopropyl-5-methylpyrimidin-4-amine

Prepared from methyl diethylmalonate and cyclopropylcarbamidine hydrochloride according to the procedures of Descriptions 48, 49, and 16 respectively. ¹H NMR (500 MHz, DMSO-d₆) 0.87 (4 H, m), 1.84 (1 H, quintet, J 6.1), 2.03 (3 H, s), 6.89 (2 H, br s).

### Description 61 2-Cyclopropyl-5-methyl-6-quinolin-7-ylpyrimidin-4-amine

Prepared from Description 60 and Description 11 according to the procedure of Description 1 to give a brown solid (1.1 g, 79%). ¹H NMR (500 MHz, CDCl₃) 0.94 (2 H, m), 1.09 (2 H, m), 2.08 (1 H, m), 2.13 (3 H, s), 4.90 (2 H, br s), 7.44 (1 H, dd, J 8.3 and 4.2), 7.76 (1 H, d, J 8.4), 7.89 (1 H, d, J 8.4), 8.19 (2 H, br s), 8.95 (1 H, s).

### Description 62 6-Chloro-5-isopropylpyrimidin-4-amine

Prepared from diethylisopropylmalonate and formamidine acetate according to the procedures of Descriptions 48, 49, and 16 respectively. ¹H NMR (360 MHz, DMSO-d₆) 1.28 (6 H, d, J 7.4), 3.35 (1 H, quintet, J 7.4), 7.01 (2 H, br s), 8.04 (1 H, s).

### Description 63 5-Isopropyl-6-quinolin-7-ylpyrimidin-4-amine

Prepared from Description 62 and Description 11 according to the procedure of Description 1 to give an off-white solid (900 mg, 68%). ¹H NMR (400 MHz, CDCl₃) 1.30 (6 H, d, J 7.4), 3.33 (1 H, quintet, J 7.4), 5.21 (2 H, br s), 7.45 (1 H, dd, J 8.2 and 4.3), 7.62 (1 H, dd, J 8.4 and 1.7), 7.91 (1 H, d, J 8.4), 8.10 (1 H, d, J 0.6), 8.21 (1 H, dd, J 8.3 and 0.9), 8.52 (1 H, s), 8.98 (1 H, dd, J 4.3 and 2.0).

### Description 64 6-(6-Fluoroquinolin-7-yl)-5-methylpyrimidin-4-amine

Prepared from Description 38 and Description 18 according to the procedures of Descriptions 10, 11 and 14 respectively. ¹H NMR (400 MHz, DMSO-d₆) 1.90 (3 H, d, J 1.5), 5.75 (2 H, s), 7.62 (1 H, dd, J 8.4 and 4.0), 7.92 (1 H, d, J 10.4), 8.03 (1 H, d, J 7.0), 8.34 (1H, s) 8.43 (1 H, d, J=8.4 Hz), 8.94 (1 H, dd, J 4.1 and 1.6).

### Description 65 2-Quinolin-7-ylpyridin-4-amine

Prepared from 2-chloropyridin-4-amine and Description 11 according to the procedure of Description 1 to give a solid (1.2 g, 67%). ¹H NMR (400 MHz, DMSO-d₆) 6.20 (2 H, br s), 6.57 (1 H, d, J 5.1), 7.28 (1 H, s), 7.54 (1 H, dd, J 7.9 and 3.9), 8.05 (1 H, d, J 8.5), 8.21 (1 H, d, J 5.4), 8.26 (1 H, d, J 8.5), 8.39 (1 H, d, J 8.3), 8.55 (1 H, s), 8.96 (1 H, m).

### Description 66 5,6-Dichloropyrimidin-4-amine

Prepared from diethyl chloromalonate and formamidine acetate according to the procedures of Descriptions 48, 49, and 16 respectively. ¹H NMR (400 MHz, DMSO-d₆) 7.46 (1 H, br s), 7.91 (1 H, br s), 8.17 (1 H, s).

### Description 67 5-Chloro-6-quinolin-7-ylpyrimidin-4-amine

Prepared from Description 66 and Description 11 according to the procedure of Description 1 to give an off-white solid (1.1 g, 61%). ¹H NMR (500 MHz, DMSO-d₆) 7.49 (2 H, br s), 7.62 (1 H, s), 7.90 (1 H, d, J 7.4), 8.10 (1 H, d, J 7.6), 8.35 (1 H, s), 8.45 (3 H, m), 8.98(1 H, s).

### Description 68 5-tert-Butyl-6-chloropyrimidin-4-amine

Prepared from diethyl tert-butylmalonate and formamidine acetate according to the procedure of Descriptions 48, 49, and 16 respectively. ¹H NMR (500 MHz, DMSO-d₆) 1.49 (9 H, s), 6.85 (2 H, br s), 7.94(1 H, s).

### Description 69 5-tert-Butyl-6-quinolin-7-ylpyrimidin-4-amine

Prepared from Description 68 and Description 11 according to the procedure of Description 1 to give an off white solid (980 mg, 70%). ¹H NMR (500 MHz, CDCl₃) 1.25 (9 H, s), 5.60 (2 H, br s), 7.41 (1 H, dd, J 8.3 and 4.2), 7.59 (1 H, dd, J 8.3 and 1.6), 7.86 (1 H, d, J 8.3), 7.97 (1 H, s), 8.18 (1 H, d, J 8.0), 8.39 (1 H, s), 8.98 (1 H, dd, J 4.2 and 1.7).

### Description 70 6-(8-Ethylquinolin-7-yl)pyrimidin-4-amine

Prepared from 2-ethyl-3-methoxyaniline [Journal of Organic Chemistry (1988), 53(12), 2844-7], according to the procedures of Descriptions 34, 35, 36, 26, 27, 10, 11, and 14 respectively to give an off white solid.

### Description 71 6-(8-Ethylquinolin-7-yl)-5-methylpyrimidin-4-amine

Prepared from 2-ethyl-3-methoxyaniline [Journal of Organic Chemistry (1988), 53(12), 2844-7], according to the procedures of Descriptions 34, 35, 36, 26, 27, 10, 11, and 19 respectively to give an off white solid. ¹H NMR (500 MHz, DMSO-d₆) 1.06 (3 H, t, J 7.5), 1.77 (3 H, s), 2.88 and 3.20 (2 H, m), 5.75 (2 H, br s), 7.32 (1 H, d, J 8.3), 7.57 (1 H, dd, J 8.2 and 4.3), 7.87 (1 H, d, J 8.4), 8.33 (1 H, s), 8.39 (1 H, dd, J 8.3 and 1.5), 8.98 (1 H, d, J 1.6).

### Description 72 Sodium 6-hydroxy-5-methyl-4-oxo-1,4-dihydropyrimidine-2-thiolate

A mixture of diethyl methylmalonate (50 g, 287 mmol) and thiourea (21.85 g, 287 mmol) in ethanol (100 ml) was stirred at room temperature for 20 min. To this mixture was added a solution of sodium (6.6 g, 287 mmol) which had been dissolved in ethanol (100 ml) and the resulting mixture heated at reflux for 4 hours (a thick white precipitate soon formed). The mixture was cooled to room temperature and filtered, the solid was washed with ethanol, and dried to give the title compound (51 g, 98%) ¹H NMR (500 MHz, D₂O) 1.73 (3 H, s), 4.78 (2 H, br s).

### Description 73 6-Hydroxy-5-methyl-2-methylthiopyrimidin-4(1)-one

To a solution of Description 72 (51.0 g, 286 mmol) in anhydrous DMF (450 ml) was added iodomethane (23.22 ml, 373 mmol) and the mixture stirred at room temperature overnight. The resulting white suspension was poured into water (11) and the solid filtered, azeotroped with toluene (3×) and dried to give the title compound (36 g, 72%). ¹H NMR (500 MHz, DMSO-d₆) 1.72 (3 H, s), 2.47 (3 H, s), 11.60 (2 H, br s).

### Description 74 4,6-Dichloro-5-methyl-2-methylthiopyrimidine

To a mixture of Description 73 (36 g, 209 mmol) and phosphorous oxychloride (390 ml, 4.18 mol) was added N,N-diethylisopropylamine (40.34 ml, 230 mmol) and the resulting mixture heated at 100° C. overnight. The excess phosphorous oxychloride was removed by evaporation, and the residue dissolved in DCM (300 ml), and poured onto ice/water (500 ml). The mixture was stirred for 30 min, the organic layer was separated, and the aqueous phase extracted with a further portion of DCM (300 ml). The combined DCM layers were washed with water (500 ml), dried over Na₂SO₄, filtered and evaporated to give the title compound as a brown solid (40 g, 91%). ¹H NMR (500 MHz, CDCl₃) 2.38 (3 H, s), 2.54 (3 H, s).

### Description 75 6-Chloro-5-methyl-2-methylthiopyrimidin-4-amine

Prepared from Description 74 according to the procedure of Description 16 to give a white solid (6.17 g, 68%). ¹H NMR (400 MHz, DMSO-d₆) 2.03 (3 H, s), 2.40 (3 H, s), 7.13 (2 H, br s).

### Description 76 6-Chloro-2-trifluoromethylpyrimidin-4-amine

Prepared from 4,6-dichloro-2-trifluoromethylpyrimidine [U.S. Pat. No. 4,963,678] according to the procedure of Description 16 to give a yellow solid (5 g, 61%). ¹H NMR (400 MHz, DMSO-d₆) 6.66 (1 H, s), 7.82 (1 H, br s), 7.97 (1 H, br s).

### Description 77 6-(8-Methylquinolin-7-yl)-2-trifluoromethylpyrimidin-4-amine

Prepared from Description 43 and Description 76 according to the procedure of Description 1 to give a white solid (210 mg, 27%). ¹H NMR (400 MHz, DMSO-d₆) 2.78 (3 H, s), 6.80 (1 H, s), 7.60-7.64 (2 H, m), 7.67 (2 H, br s), 7.93 (1 H, d, J 8.4), 8.41(1 H, d, J 8.4), 9.02 (1 H, d, J 1.9).

### Description 78 Ethyl 2-methoxyethanimidoate Hydrochloride

Hydrogen chloride gas was bubbled through a solution of methoxyacetonitrile (50 g, 703 mmol) in a mixture of ethanol (41 ml) and diethyl ether (250 ml) at −15° C. until the mixture was saturated. The mixture was stirred for 1 hour at −15° C. then the solid which had formed was removed by filtration, washed with ether, and dried under a stream of nitrogen to give the title compound as a white solid (90 g, 83%). ¹H NMR (400 MHz, DMSO-d₆) 1.35 (3 H, t, J 7.0), 3.38 (3 H, s), 4.42 (2 H, s), 4.56 (2 H, q, J 7.0), 11.70 (2 H, br s).

### Description 79 2-Methoxyethanimidamide Hydrochloride

Ethanol (500 ml) was cooled to −15° C. and anhydrous ammonia gas passed through until the mixture was saturated. Description 78 (90 g, 585 mmol) was added and the mixture stirred at room temperature overnight. The mixture was recooled to −15° C. and a small amount of solid removed by filtration. The filtrate was evaporated to dryness and the residue crystallised on standing to give the title compound (70 g, 96%). ¹H NMR (360 MHz, DMSO-d₆) 3.35 (3 H, s), 4.24 (2 H, s), 8.85 (4 H, br s).

### Description 80 6-Chloro-2-methoxymethyl-5-methylpyrimidin-4-amine

Prepared from Description 79 and methyl diethylmalonate according to the procedures of Descriptions 48, 49 and 16 respectively to give a white solid. ¹H NMR (400 MHz, DMSO-d₆) 2.06 (3 H, s), 3.30 (3 H, s), 4.22 (2 H, s), 7.10 (2 H, br s).

### Description 81 2-Methoxymethyl-5-methyl-6-quinolin-7-ylpyrimidin-4-amine

Prepared from Description 11 and Description 80 according to the procedure of Description 1 to give a dark solid (4.5 g, 75%). ¹H NMR (500 MHz, CDCl₃) 2.17 (3 H, s), 3.53 (3 H, s), 4.56 (2 H, s), 5.37 (2 H, br s), 7.44 (1 H, dd, J 8.4 and 4.2), 7.75 (1 H, dd, J 8.4 and 1.6), 7.91 (1 H, d, J 8.4), 8.20 (2 H, m), 8.97 (1 H, d, J 1.7).

### Description 82 5-Fluoro-6-(8-fluoroquinolin-7-yl)pyrimidin-4-amine

Prepared from Description 40 according to the procedures of Descriptions 34, 35, 36, 26, 27, 10, 11 respectively, then the resulting compound treated with Description 20 according to the procedure of Description 1. ¹H NMR (400 MHz, DMSO-d₆) 7.60 (2 H, br s), 7.74-7.81 (2 H, m), 7.98 (1 H, d, J 8.2), 8.37 (1 H, s), 8.55 (1 H, d, J 7.8), 9.06 (1 H, s).

### Description 83 4-Isopropyl-2-trifluoromethyl-1,3-oxazol-5(2H)-one

Trifluoroacetic anhydride (120 ml, 854 mmol) was cooled in an ice bath, and DL-Valine (50.0 g, 427 mmol) was added portionwise. After complete addition the mixture was heated at 80° C. for 30 min, then at 130° C. for 30 min. The temperature was kept at 130° C., and excess trifluoroacetic anhydride and trifluoroacetic acid were distilled off. The residue was partitioned between diethyl ether and water, the ether layer washed with sat. NaHCO₃, dried over Na₂SO₄ , filtered and evaporated. The residue was distilled under vacuum to give the title compound (b.p. 56-58° C. @ 11 mmHg ) as a clear oil (54 g, 65%). ¹H NMR (500 MHz, CDCl₃) 1.18 (6 H, m), 2.91 (1 H, m), 5.93 (1 H, m).

### Description 84 tert-Butyl 3-[4-isopropyl-5-oxo-2-trifluoromethyl-2,5-dihydro-1,3-oxazol-2-yl]propanoate

To Description 83 (54.4 g, 279 mmol) dissolved in anhydrous dichloromethane (150 ml) and cooled in an ice bath, was added tert-butyl acrylate (49.0 ml, 334.8 mmol) followed by triethylamine (48.6 ml, 348.75 mmol), and the resulting mixture stirred at room temperature overnight. The mixture was washed with 10% citric acid solution (500 ml), sat. NaHCO₃, sat. NaCl, dried over Na₂SO₄ , filtered and evaporated to give the title compound as a pale yellow oil (90 g, 100%). ¹H NMR (400 MHz, CDCl₃) 1.32 (6 H, t, J 6.9), 1.44 (9 H, s), 2.09 (2 H, t, J 7.8), 2.53 (2 H, m), 3.05 (1 H, septet, J 6.9).

### Description 85 6-Trifluoromethyl-4,5-dihydropyridazin-3(2H)-one

A mixture of Description 84 (90.2 g, 279 mmol) and hydrazine hydrochloride (95.6 g, 1.4 mol) in glacial acetic acid (500 ml) was heated at reflux for 2 hours. The cooled reaction mixture was evaporated, and the residue basified by the careful addition of saturated aqueous K₂CO₃. Water (500 ml) was added, and the mixture extracted with dichloromethane (×3). The combined dichloromethane layers were dried over Na₂SO₄ , filtered and evaporated to give the title compound as an oil which crystallised on standing (50 g, quant). ¹H NMR (400 MHz, CDCl₃) 2.62 (2 H, m), 2.78 (2 H, m), 9.57 (1 H, br s).

### Description 86 6-Trifluoromethylpyridazin-3(2H)-one

To a solution of Description 85 (46.34 g, 279 mmol) in glacial acetic acid (300 ml) warmed at 100° C., was added dropwise a solution of bromine (14.29 ml, 279 mmol). After complete addition the heating was continued for 4 hours. The acetic acid was removed by evaporation and the residue partitioned between dichloromethane and water. The organic layer was washed with sat. NaHCO₃, sat. NaCl, dried over Na₂SO₄, filtered and evaporated. The dark residue was triturated with diethyl ether, and the solid filtered, and dried to give the title compound as a white solid (5 g, 11%). ¹H NMR (400 MHz, CDCl₃) 7.14 (1 H, d, J 9.6), 7.53 (1 H, d, J 9.6), 12.65 (1 H, br s).

### Description 87 3-Chloro-6-trifluoromethylpyridazine

A mixture of Description 86 (2.00 g, 12.2 mmol) and phosphorous oxychloride (11.4 ml, 122 mmol) was heated at 90° C. for 6 hours. The excess phosphorous oxychloride was removed by evaporation, and the residue then dissolved in dichloromethane (100 ml) and ice (100 g) added. The mixture was stirred for 30 min, then carefully basified by the addition of saturated aqueous K₂CO₃, filtered and evaporated to give the title compound as a pale brown solid (2 g, 89%). ¹H NMR (400 MHz, CDCl₃) 7.79 (1 H, d, J 9.0), 7.86(1 H, d, J 9.0).

### Description 88 6-Chloro-5-methyl-N-[5-trifluoromethylpyridin-2-yl]pyrimidin-4-amine

Prepared from Description 18 and 2-chloro-5-trifluoromethylpyridine according to the procedure of Description 7 to give an off white solid (4 g, 66%). ¹H NMR (400 MHz, CDCl₃) 2.40 (3 H, s), 7.61 (1 H, br s), 7.94 (1 H, dd, J 8.8 and 2.2), 8.52 (1 H, s), 8.54 (1 H, s), 8.65 (1 H, d, J 8.8).

### Description 89 5-Bromo-4-chloro-7-fluoroquinoline and 7-bromo-4-chloro-5-fluoroquinoline

Prepared from 3-bromo-5-fluoroaniline [WO-A-9215565] according to the procedures of Descriptions 34, 35, and 36 respectively to give a mixture of the title compounds as an off white solid.

### Description 90 7-Bromo-5-fluoroquinoline

A mixture of Description 89 (8 g, 30.7 mmol) and hydrazine hydrate (7.46 ml, 153.5 mmol) in ethanol (100 ml) was heated at reflux for 4 hours. The cooled mixture was filtered and the filtrate evaporated. The residue was suspended in chloroform (100 ml) and manganese dioxide (10.9 g, 153.5 mmol) added in a portionwise manner. After complete addition the mixture was heated at reflux for 4 hours. The mixture was cooled and filtered through Hyflo supercel™, the filter cake was washed with methanol, and the combined filtrates evaporated. The mixture was purified and the isomers separated by column chromatography on silica (eluent: 2% MeOH in DCM), and further purified by mass directed HPLC to give the title compound as a light brown solid (200 mg, 3%). ¹H NMR (500 MHz, CDCl₃) 7.37 (1 H, dd, J 9.1 and 1.5), 7.47 (1 H, dd, J 8.3 and 4.2), 8.11 (1 H, s), 8.38 (1 H, d, J 8.3), 8.95 (1 H, dd, J 4.2 and 1.5).

### Description 91 5-Methyl-2-methylthio-6-quinolin-7-ylpyrimidin-4-amine

Prepared from Description 11 and Description 75 according to the procedure of Description 1 to give an off white solid (2.5 g, 56%). ¹H NMR (400 MHz, CDCl₃) 2.15 (3 H, s), 2.54 (3 H, s), 5.06 (2 H, br s), 7.44 (1 H, dd, J 8.2 and 4.2), 7.79 (1 H, dd, J 8.4 and 1.5), 7.90 (1 H, d, J 8.4), 8.20 (1 H, d, J 8.0), 8.23 (1 H, s), 8.97 (1 H, dd, J 4.2 and 1.5).

### Description 92 2-Chloro-1,8-naphthyridine

Phosphorous oxychloride (86 ml, 924 mmol) was added to 1,8-naphthyridin-2-one [Journal of Organic Chemistry 1990, 55(15), 4744-50] (9.00 g, 61.6 mmol), and the resulting mixture heated to 100° C. for 1 hour. The mixture was cooled and the excess phosphorous oxychloride was removed by evaporation. The residue was taken up in dichloromethane (100 ml) and carefully basified by the addition of sat. NaHCO₃. The organic layer was separated and dried over Na₂SO₄, filtered, and evaporated to give the title compound as a white solid (7 g, 70%). ¹H NMR (400 MHz, CDCl₃) 7.50 (1 H, d, J 8.4), 7.52 (1 H, dd, J 8.1 and 4.3), 8.16 (1 H, d, J 8.4), 8.22 (1 H, dd, J 8.1 and 2.0), 9.12 (1 H, dd, J 4.3 and 2.0).

### Description 93 6-Iodopyrimidin-4-amine

A mixture of 4-amino-6-chloropyrimidine [WO-A-0245652] (1.00 g, 7.72 mmol), sodium iodide (5.79 g, 38.6 mmol) and 40% HI (20 ml) were heated at 70° C. for 30 min, then allowed to cool to room temperature. The precipitate was removed by filtration, and partitioned between dichloromethane, and sat. NaHCO₃. The organic layer was separated, and dried over Na₂SO₄, filtered, and evaporated to give the title compound (1.2 g, 70%). ¹H NMR (400 MHz, DMSO-d₆) 6.89 (1 H, s), 7.04 (2 H, br s), 8.04 (1 H, s).

### Description 94 6-(1,8-Naphthyridin-2-yl)pyrimidin-4-amine

To a mixture of Description 92 (2.52 g, 15.3 mmol), hexamethylditin (5.0 g, 15.3 mmol), lithium chloride (1.95 g, 45.9 mmol), and copper (I) iodide (291 mg, 1.53 mmol) in anhydrous 1,4-dioxane (50 ml) was added Pd(PPh₃)₄ (884 g, 0.77 mmol). The mixture was de-gassed three times, and heated at 100° C. overnight. The mixture was cooled and diluted with EtOAc (120 ml) and washed with a 10% potassium fluoride solution (200 ml). The organic layer was washed with sat. NaCl (50 ml), dried over Na₂SO₄, filtered, and evaporated. The residue was taken up in anhydrous 1,4-dioxane (75 ml), and Description 93 (1.55 g, 7 mmol), lithium chloride (1.78 g, 42 mmol), and copper (I) iodide (266 mg, 1.4 mmol) added, followed by Pd(PPh₃)₄ (808 mg, 0.7 mmol). The mixture was de-gassed 3 times and heated at 100° C. for 3 days. The mixture was poured into water (200 ml), and extracted with EtOAc (2×100 ml), the combined EtOAc layers were washed with water (150 ml), sat. NaCl (100 ml), dried over Na₂SO₄, filtered and evaporated. The residue was purified by column chromatography on silica (eluent: 2% MeOH in DCM+0.5% NH₄OH) to give the title compound (100 mg, 3%). ¹H NMR (360 MHz, DMSO-d₆) 7.18 (2 H, br s), 7.66-7.86 (3 H, m), 8.55 (1 H, dd, J 8.1 and 1.8), 8.58 (1 H, d, J 4.2), 8.64 (1 H, d, J 8.4), 9.16 (1 H, dd, J 4.2 and 2.1).

### Description 95 6-Chloro-5-methyl-N-[4-trifluoromethylphenyl]pyrimidin-4-amine

A mixture of 4,6-dichloro-5-methylpyrimidine (1.0 g, 6.15 mmol) and 4-aminobenzotrifluoride (0.77 ml, 6.15 mmol) in ethanol (12 ml) was heated at 150° C. for 15 min in a microwave reactor (Personal Chemistry—Emrys Optimizer). The solid which had formed was removed by filtration and dried to give the title compound (950 mg, 53%). ¹H NMR (400 MHz, CDCl₃) 2.36 (3 H, s), 6.70 (2 H, br s), 7.61 (2 H, d, J 8.6), 7.73 (2 H, d, J 8.6), 8.44 (1 H, s).

### Description 96 6-Chloro-5-isopropyl-N-[4-trifluoromethylphenyl]pyrimidin-4-amine

Prepared from Description 62 and 4-trifluoromethylbromobenzene according to the procedure of Description 7 (640 mg, 67%). ¹H NMR (400 MHz, CDCl₃) 1.47 (6 H, d, J 7.4), 3.72 (1 H, quintet, J 7.4), 6.85 (1 H, br s), 7.61 (2 H, d, J 8.6), 7.71 (2 H, d, J 8.6), 8.39 (1 H, s).

### Description 97 5-tert-Butyl-6-chloro-N-[4-trifluoromethylphenyl]pyrimidin-4-amine

Prepared from Description 68 and 4-trifluoromethylbromobenzene according to the procedure of Description 7 (640 mg, 67%). ¹H NMR (400 MHz, CDCl₃) 1.71 (9 H, s), 7.18 (1 H, br s), 7.61 (4 H, m), 8.30 (1 H, s).

### Description 98 6-(1-{2-Trimethylsilylethoxymethyl}-1H-benzimidazol-6-yl)pyrimidin-4-amine and 6-(1-{2-trimethylsilylethoxymethyl}-1H-benzimidazol-5-yl)pyrimidin-4-amine

Prepared from a mixture of 5-(4,4,5,5-tetramethyl-1,3,2-dioxaborolan-2-yl)-1-{2-trimethylsilylethoxymethyl}-1-benzimidazole and 6-(4,4,5,5-tetramethyl-1,3,2-dioxaborolan-2-yl)-1-{2-trimethylsilylethoxymethyl}-1H-benzimidazole [WO-A-0100213], and 4-amino-6-chloropyrimidine [WO-A-0245652] according to the procedure of Description 1 (400 mg, 30%). ¹H NMR (400 MHz, CDCl₃) 0.02 (9 H, s), 0.96 (2 H, m), 3.60 (2 H, m). 5.09 (2 H, br s), 5.61 and 5.66 (2 H, s), 6.96 and 6.97 (1 H, s), 7.67 (1 H, d, J 8.5), 7.92 (1 H, s), 8.09 (1 H, s)8.13 (1 H, m), 8.36 and 8.43 (1 H, s), 8.74 (1 H, s).

### Description 99 N-[4-Trifluoromethylphenyl]-6-(1-{2-trimethylsilylethoxymethyl}-1H-benzimidazol-6-yl)pyrimidin-4-amine and N-[4-trifluoromethylphenyl]-6-(1-{2-trimethylsilylethoxymethyl}-1H-benzimidazol-5-yl)pyrimidin-4-amine

Prepared from Description 98 and 4-trifluoromethylbromobenzene according to the procedure of Description 7 (210 mg, 37%). ¹H NMR (500 MHz, CDCl₃) 0.00 (9 H, s), 0.96 (2 H, t, J 8.2), 3.58 (2 H, t, J 8.2), 5.61 (2 H, s), 7.27 (1 H, d, J 0.9), 7.32 (1 H, s), 7.69 (5 H, m), 8.09 (1 H, s), 8.16 (1 H, dd, J 8.5 and 1.5), 8.44 (1 H, d, J 1.1), 8.91 (1 H, s).

### Description 100 5-Methyl-6-quinoline-8-ylpyrimidin-4-amine

Prepared from quinoline-8-boronic acid and Description 18 according to the procedure of Description 1 (0.98 mg, 91%). ¹H NMR (500 MHz, DMSO-d₆) 1.63 (3 H, s), 6.66 (2 H, br s), 7.55 (1 H, dd, J 8.2 and 4.3), 7.65-7.70 (2 H, m), 8.05 (1 H, d, J 7.7), 8.30 (1 H, s), 8.44 (1 H, d, J 8.2), 8.83 (1 H, s).

### Description 101 7-Methoxy-4-trifluoromethylquinolin-2(1H)-one

A solution of m-anisidine (22.8 ml, 203 mmol), and ethyl 2,2,2-trifluoroacetoacetate (35.6 ml, 243.6 mmol) in toluene (500 ml) was heated at reflux for 24 hours. Toluene sulfonic acid (3.86 g, 20.3 mmol) was added, and heating continued for a further 24 hours. The cooled mixture was evaporated and the residue treated with diethyl ether and solid removed by filtration to give the title compound as a yellow solid (9 g, 18%). ¹H NMR (400 MHz, DMSO-d₆) 3.85 (3 H, s), 6.77 (1 H, s), 6.93 (2 H, m), 7.61 (1 H, dd, J 9.8 and 2.1), 12.16 (1 H, br s).

### Description 102 2-Chloro-7-methoxy-4-trifluoromethylquinoline

Prepared from Description 101 according to the procedure of Description 92 (8.63 g, 89%). ¹H NMR (400 MHz, CDCl₃) 3.96 (3 H, s), 7.31 (1 H, dd, J 9.4 and 2.7), 7.43 (1 H, d, J 2.7), 7.54 (1 H, s), 7.98 (1 H, dd, J 9.4 and 2.0).

### Description 103 7-Methoxy-4-trifluoromethylquinoline

Prepared from Description 102 according to the procedure of Description 26 (4.6 g, 61%). ¹H NMR (400 MHz, CDCl₃) 3.98 (3 H, s), 7.33 (1 H, dd, J 9.4 and 2.6), 7.51 (1 H, d, J 2.6), 7.54 (1 H, d, J 4.3), 8.03 (1 H, dd, J 9.4 and 2.0), 8.95 (1 H, d, J 4.3).

### Description 104 7-[6-Chloro-5-methyl-2-methylthiopyrimidin-4-yl]quinoline

Prepared from Description 11 (3.2 g, 12 mmol) and Description 74 (5.2 g, 24 mmol) according to the procedure of Description 1 to give a solid (2.9 g, 77%). ¹H NMR (400 MHz, CDCl₃) 2.43 (3 H, s), 2.60 (3 H, s), 7.49 (1 H, dd, J 8.3 and 4.2), 7.77 (1 H, dd, J 8.4 and 1.7), 7.94 (1 H, d, J 8.4), 8.23 (1 H, d, J 8.3), 8.29 (1 H, s), 8.98-9.00 (1 H, m).

### Description 105 Ethyl 7-methoxy-4-oxo-1,4-dihydroquinoline-3-carboxylate

A solution of 3-methoxyaniline (30 ml, 0.27 mol) and diethylethoxymethylenemalonate (54 ml, 0.27 mol) was heated at 100° C. for 3hrs. The cooled reaction mixture was slowly added to boiling Dowtherm® A (300 ml). The reaction mixture was stirred until gas evolution had ceased 30 min. The cooled reaction mixture was poured into hexane and the solid which formed was collected by filtration and washed with hexane to give the title compound (28.6 g, 43%). ¹H NMR (400 MHz, DMSO-d₆) 1.27 (3 H, t, J 7.1), 3.87 (3 H, s), 4.20 (2 H, q, J 7.1), 7.00 (2 H, m), 8.05 (1 H, d, J 9.5), 8.48 (1 H, s).

### Description 106 Ethyl 4-chloro-7-methoxyquinoline-3-carboxylate

A suspension of Description 105 (28.6 g, 0.12 mol) in POC₃ (34 ml, 0.36 mol) was heated at 115° C. for 45 mins. The cooled reaction mixture was poured onto ice (500 ml) and cooled in an ice bath. 33% aqueous ammonia was added until a pH of 7 was obtained (80-90 ml). The solid which formed was collected by filtration. Ether was added to the solid, the mixture stirred then filtered. This procedure was repeated 4 times and the combined ether extracts were evaporated to give a yellow solid (11.1 g, 36%). ¹H NMR (400 MHz, DMSO-d₆) 1.38 (3 H, t, J 7.1), 3.99 (3 H, s), 4.41 (2 H, q, J 7.1), 7.48 (1 H, dd, J 9.3 and 2.6), 7.53 (1 H, d, J 2.5), 8.28 (1 H, d, J 9.2), 9.10 (1 H, s).

### Description 107 4-Chloro-7-methoxyquinoline-3-carboxylic acid

To a stirring suspension of Description 106 (11.1 g, 41 mmol) in ethanol (100 ml) was added 2M NaOH (100 ml). The reaction mixture was stirred at room temperature for 3 days. The reaction mixture was diluted with water (100 ml), cooled in an ice/water bath and acidified to pH 4 by addition of conc. HCl. The resulting solid was collected by filtration, washed with water and dried under vacuum in a drying pistol at 50° C. (8.8 g, 89%). ¹H NMR (400 MHz, DMSO-d₆) 3.98 (3 H, s), 7.48 (1H, dd, J 9.2 and 2.5), 7.52 (1 H, d, J 2.5), 8.28 (1 H, d, J 9.2), 9.11 (1 H, s).

### Description 108 and 109 tert-Butyl (4-chloro-7-methoxyquinolin-3-yl)carbamate (Description 108) and 4-chloro-7-methoxyquinolin-3-amine (Description 109)

To a stirred suspension of Description 107 (8.8 g, 37 mmol) in DMF (400 ml) under N₂, was added tert-butanol (150 ml) and triethylamine (12 ml, 86 mmol), followed by diphenylphosphorylazide (9.5 ml, 44 mmol). The mixture was heated at 100° C. for 3hours. The cooled reaction mixture was then evaporated and the residue was purified by column chromatography on silica (eluant: DCM to 4% MeOH in DCM). This gave Description 108 as a solid (2.12 g, 20%) and Description 109 as an orange solid (4.26 g, 55%). Description 108 ¹H NMR (360 MHz, DMSO-d₆) 1.48 (9 H, s), 3.94 (3 H, s), 7.40 (1 H, dd, J 9.2 and 2.6), 7.47 (1 H, d, J 2.4), 8.07 (1 H, d, J 9.2), 8.85 (1 H, s). Description 109 ¹H NMR (360 MHz, DMSO-d₆) 3.86 (3 H, s), 5.76 (2 H, s), 7.25 (1 H, dd, J 9.1 and 2.6), 7.30 (1 H, d, J 2.5), 7.80 (1 H, d, J 9.1), 8.52 (1 H, s).

### Description 110 4-Chloro-3-fluoro-7-methoxyquinoline

A solution of Description 109 (4.26 g, 20 mmol) in THF (100 ml) was cooled in an ice/water bath and 48% fluoroboric acid (11 ml, 60 mmol) was carefully added. The mixture was stirred for 5min, and then a solution of sodium nitrite (1.55 g, 22 mmol) in water (3 ml) was added dropwise keeping the temperature of the reaction below 10° C. The mixture was stirred for 30 minutes in an ice/water bath. The resulting yellow solid was filtered and washed with THF. The solid was then heated at 170° C. until gas evolution had ceased. The residue was purified by column chromatography on silica (eluant: 1% MeOH in DCM) to give a cream solid (780 mg, 18%). ¹H NMR (400 MHz, DMSO-d₆) 3.95 (3 H, s), 7.49 (1 H, dd, J 9.0 and 2.5), 7.53 (1 H, d, J 2.5), 8.09 (1 H, d, J 9.3), 9.00 (1 H, d, J 1.0).

### Description 111 3-Fluoro-7-(4,4,5,5-tetramethyl-1,3,2-dioxaborolan-2-yl)quinoline

Prepared from Description 110 according to the procedures of Descriptions 26, 27, 10, and 11 respectively to give a solid. ¹H NMR (400 MHz, DMSO-d₆) 1.35 (12 H, s), 7.85 (1 H, d, J 7.8), 7.94 (1 H, s), 8.00 (1 H, d, J 8.2), 8.36 (1 H, s), 8.99 (1 H, d, J 2.8).

### Description 112 6-(3-Aminophenyl)-5-methyl-N-[5-trifluoromethylpyridin-2-yl]pyrimidin-4-amine

Prepared from Description 88 and (3-aminophenyl)boronic acid according to the procedure of Description 1 to give a light brown solid (1.58 g, 74%). ¹H NMR (400 MHz, DMSO-d₆) 2.29 (3 H, s), 5.23 (2 H, s), 6.64-6.66 (2 H, m), 6.75 (1 H, t, J 1.9), 7.12 (1 H, t, J7.8), 8.14 (1 H, dd, J 9.0 and 2.4), 8.32 (1 H, d, J 8.9), 8.69 (2 H, s), 9.47 (1 H, s).

### Description 113 2,2-Dimethyl-5-(3-(5-methyl-6-5-trifluoromethylpyridin-2-ylamino}pyrimidin-4-yl)phenylaminomethylene)-1,3-dioxane-4,6-dione

Prepared from Description 112 according to the procedure of Description 34 to give a solid (1.13 g, 100%). ¹H NMR (360 MHz, DMSO-d₆) 1.68 (6 H, s), 2.32 (3 H, s), 7.46 (1 H, d, J 7.7), 7.57 (1 H, t, J 7.8), 7.69 (1 H, d, J 8.0), 7.77 (1 H, s), 8.17 (1 H, dd, J 9.0 and 2.5), 8.35 (1 H, d, J 8.9), 8.62 (1 H, d, J 14.6), 8.71 (1 H, s), 8.75 (1 H, s), 9.61 (H, s), 11.36 (1 H, d, J 14.6).

### Description 114 4-Chloro-6-quinolin-7-ylpyrimidin-5-amine

Prepared from 4,6-dichloropyrimidin-5-amine and Description 11 according to the procedure of Description 1 to give a solid (1.5 g, 65%). ¹H NMR (360 MHz, DMSO-d₆) 5.79 (2H, s), 7.62 (1H, dd, J 8.4 and 4.2), 7.92 (1H, dd, J 8.6 and 1.6), 8.13 (1H, d, J 8.4), 8.39 (2H, s), 8.45 (1H, d, J 8.4), 8.99 (1H, dd, J 4.2 and 1.8).

### Description 115 3-Chloro-5-trifluoromethylpyridine-2-carbonitrile

To 3-chloro-2-fluoro-5-trifluoromethylpyridine (10 g, 50 mmol) in DMSO (70 ml) was added potassium cyanide (3.6 g, 55 mmol) over 20 min, ensuring the reaction mixture temperature stayed below 30° C. The reaction mixture was stirred for a further 30 minutes then poured onto ice water (150 ml). The mixture was extracted 3 times with hexane, and the combined organic extracts were evaporated to give a solid (8.7 g, 84%). ¹H NMR (400 MHz, CDCl₃) 8.15 (1 H, d, J 1.2), 8.88 (1 H, d, J 1.0).

### Description 116 3-Fluoro-5-trifluoromethylpyridine-2-carbonitrile

A suspension of cesium fluoride (9.6 g, 63 mmol) and potassium carbonate (250 mg, 1.8 mmol) in anhydrous DMSO (50 ml) under N₂ was heated to 80° C. and Description 115 (8.7 g, 42 mmol) was added over 10 min. The reaction mixture was then heated to 95° C. for 20 min, cooled to 55° C. and poured into ice water. The mixture was extracted twice with hexane and once with DCM. The combined organic extracts were evaporated to give a solid (8 g, 100%). ¹H NMR (400 MHz, CDCl₃) 7.91 (1 H, d, J 7.6), 8.84 (1 H, s).

### Description 117 3-Fluoro-5-trifluoromethylpyridine-2-carboxamide

Description 116 (8 g, 42 mmol) was added to stirring conc. H₂SO₄ (65 ml) and the reaction mixture heated at 105° C. for 2 hours, then cooled to room temperature and poured onto ice water. The solid which formed was collected by filtration and washed with a little water to give the title compound (5.8 g, 66%). ¹H NMR (400 MHz, DMSO-d₆) 7.90 (1 H, s), 8.17 (1 H, s), 8.46 (1 H, d, J 10.1), 8.88 (1 H, s).

### Description 118 3-Fluoro-5-trifluoromethylpyridin-2-amine

Bromine (630 μl, 1.3 mmol) was added to a solution of KOH (3.3 g, 58 mmol) in water (30 ml) cooled at 0° C. The resulting yellow solution was stirred for 5 minutes, then Description 117 (2 g, 9.6 mmol) was added in portions over 2 hours and the reaction mixture stirred at room temperature for 5 days. The mixture was then extracted 3 times with diethyl ether and the combined organic extracts dried over Na₂SO₄, filtered, and evaporated to give a solid (330 mg, 20%). ¹H NMR (400 MHz, CDCl₃) 5.07 (2 H, s), 7.40 (1 H, dd, J 10.4 and 1.6), 8.14 (1 H, s).

### Description 119 7-(6-Chloro-5-methylpyrimidin-4-yl)quinoline

Prepared from 4,6-dichloro-5-methylpyrimidine and Description 11 according to the procedure of Description 1 to give a solid (2.3 g, 58%). ¹H NMR (400 MHz, CDCl₃) 2.53 (3 H, s), 7.50 (1 H, dd, J 8.6 and 4.3), 7.79 (1 H, dd, J 8.3 and 1.8), 7.98 (1 H, d, J 8.4), 8.24 (1 H, d, J 8.4), 8.28 (1 H, s), 8.94 (1 H, s), 9.00 (1 H, dd, J 4.2 and 1.6).

### Description 120 6-[4-Trifluoromethylquinolin-7-yl]pyrimidin-4-amine

Prepared from Description 103 according to the procedures of Descriptions 27, 10, and 11 respectively then reaction of the product with 6-chloropyrimidin-4-amine according to the procedure of Description 1 to give a solid. ¹H NMR (500 MHz, CDCl₃) 5.03 (2 H, s), 5.65 (1H, s), 7.04 (1 H, s), 7.74 (1 H, d, J 4.1), 8.25 (1 H, d, J 8.6), 8.27 (1 H, s), 8.39 (1 H, d J 8.8), 9.10 (1 H, d, J 4.2).

### Description 121 5-Methyl-6-[4-trifluoromethylquinolin-7-yl]pyrimidin-4-amine

Prepared from Description 103 according to the procedures of Descriptions 27, 10, and 11 respectively then reaction of the product with 6-chloro-5-methylpyrimidin-4-amine according to the procedure of Description 1 to give a solid. ¹H NMR (400 MHz, DMSO-d₆) 2.09 (3 H, s), 6.88 (2 H, s), 7.96-8.02 (2 H, m), 8.19 (1 H, dd, J 8.8 and 2.1), 8.30 (1 H, d, J 1.7), 8.36 (1 H, s), 9.19 (1 H, d, J 4.3).

### Description 125 4-Chloro-2-methyl-6-quinolin-7-ylpyrimidin-5-amine

Prepared from 4,6-dichloro-2-methylpyrimidin-5-amine and Description 11 according to the procedure of Description 1 to give a grey solid (64 mg, 17%). ¹H NMR (400 MHz, DMSO-d₆) 2.50 (3H, s), 5.47 (2 H, s), 7.61 (1 H, dd, J 8.2 and 4.1), 7.90 (1 H, dd, J 8.5 and 1.8), 8.12 (1 H, d, J 8.4), 8.37 (1 H, s), 8.44 (1 H, d, J 7.4), 8.98 (1 H, dd, J 4.2 and 1.7).

### Description 126 4-Chloro-6-methyl-N-[4-trifluoromethylphenyl]-1,3,5-triazin-2-amine

To a solution of 2,4-dichloro-6-methyltriazine [J Med Chem 805-18 (1999)] (0.3 g, 1.8 mmol) in anhydrous dioxane (4 ml) was added diisopropylethylamine (0.3 g, 2.3 mmol) followed by 4-trifluoromethylaniline (0.3 g, 1.9 mmol) and the resulting mixture heated at 140° C. for 200 secs in a microwave apparatus. The mixture was diluted with ethyl acetate (15 ml) and washed with 2N HCl (10 ml), sat. NaCl (10 ml), dried over MgSO₄, filtered and evaporated to give the title compound as a white solid after trituration with ether (0.25 g, 48%). ¹H NMR (360 MHz, CDCl₃)2.56 (3 H, s), 7.26 (1 H, s), 7.64 (2 H, d, J 8.6), 7.75 (2 H, d, J 8.6).

### Description 127 4-Chloro-2-(1,1-dimethylethyl)-5-methyl-6-quinolin-7-ylpyrimidine

Prepared from 4,6-dichloro-2-(1,1-dimethylethyl)-5-methylpyrimidine and Description 11 according to the procedure of Description 1 to give a yellow oil (110 mg). ¹H NMR (360 MHz, CDCl₃)1.44 (9 H, s), 2.49 (3 H, s), 7.49 (1 H, dd, J 8.4 and 4.2), 7.83 (1 H, dd, J 8.4 and 1.6), 7.95 (1 H, d, J 8.4), 8.22 (1 H, d, J 8.4), 8.34 (1 H, s), 9.0 (1 H, d, J 1.6).

### Description 128 4,6-Dichloro-2-iodomethylpyrimidine

A mixture of 4,6-dichloro-2-chloromethylpyrimidine [Annales Pharmaceutici (Poznan) 12, 33-38, 1977] (3.3 g, 16.7 mmol) and sodium iodide (3.25 g, 21.7 mmol) in acetone (70 ml) was stirred at room temperature for 3 hours. The reaction mixture was concentrated to dryness. The residue was dissolved in ethyl acetate and the organic solution was washed with sodium thiosulfate solution (aq), brine, dried over sodium sulfate, filtered and concentrated to give a brown solid (4.5 g, 93%). ¹H NMR (360 MHz, DMSO-d₆) 4.53 (2 H, s), 7.93 (1 H, s).

### Description 129 4-(4,6-Dichloropyrimidin-2-yl)methylmorpholine

Description 128 (1.0 g, 3.46 mmol), morpholine (301 □1, 3.46 mmol) and potassium carbonate (1.43 g, 10.4 mmol) in DMF (15 ml) were stirred at room temperature for 18 hours. The mixture was diluted with ethyl acetate and washed with water and brine, dried over sodium sulphate, filtered and concentrated. The residue was purified by flash chromatography on silica gel eluting with 40:1 DCM-MeOH to give an orange solid (318 mg, 37%). ¹H NMR (360 MHz, CDCl₃) 2.63 (4H, m), 3.77 (6H, m), 7.32 (1 H, s).

### Description 130 6-Chloro-2-(morpholin-4-ylmethyl)-N-[4-trifluoromethylphenyl]pyrimidin-4-amine

A mixture of Description 129 (252 mg, 1.02 mmol) and 4-trifluoromethylaniline (128 □1, 1.02 mmol) in ethanol (3 ml) was heated at 160° C. for 90 mins in a microwave apparatus. The cooled mixture was diluted with ethyl acetate, washed with sodium carbonate solution (aq), dried over sodium sulphate, filtered and concentrated. The residue was purified by flash chromatography on silica gel eluting with 2:1 ethyl acetate-isohexane. The product was then triturated with a mixture of diethyl ether and isohexane to give a pale brown solid (123 mg, 33%). ¹H NMR (500 MHz, CDCl₃) 2.65 (4 H, m), 3.68 (2 H, s), 3.80 (4 H, m), 6.69 (1 H, s), 7.06 (1 H, br s), 7.48 (2 H, d, J 8.4), 7.66 (2 H, d, J 8.5).

### Description 131 (4,6-Dichloropyrimidin-2-yl)methyl acetate

A mixture of Description 128 (1.0 g, 3.46 mmol) and potassium acetate (339 mg, 3.46 mmol) in 50% aqueous dioxane (40 ml) was stirred and heated at 60° C. for 18 hours. Further potassium acetate (339 mg, 3.46 mmol) was added and the mixture was stirred and heated at 60° C. for a further 18 hours. The cooled mixture was diluted with ethyl acetate, washed with water, dried over sodium sulphate, filtered and concentrated. The residue was purified by flash chromatography on silica gel eluting with 10:1 isohexane-ethyl acetate to give a colourless oil (600 mg, 78%). ¹H NMR (400 MHz, CDCl₃) 2.22 (3 H, s), 5.24 (2 H, s), 7.32 (1 H, s).

### Description 132 (4-Chloro-6-{4-trifluoromethylphenylamino}pyrimidin-2-yl)methanol

Prepared from Description 131 and 4-trifluoromethylaniline according to the procedure of Description 130 to give a white solid (88 mg, 20%). ¹H NMR (360 MHz, DMSO-d₆) 4.47 (2 H, d, J 6.0), 5.33 (1 H, t, J 6.4), 6.78 (1 H, s), 7.68 (2 H, d, J 8.6), 7.96 (2 H, d, J 8.5), 10.20 (1 H, s).

### Description 133 7-(6-Chloro-2-isopropyl-5-methylpyrimidin-4-yl)quinoline

Prepared from 4,6-dichloro-2-isopropyl-5-methylpyrimidine [WO-A-03087064] and Description 11 according to the procedure of Example 109 except that the reaction mixture was stirred and heated under reflux for 18 hours. ¹H NMR (500 MHz, DMSO-d₆) 1.32 (6 H, d, J 6.9), 2.40 (3 H, s), 3.18-3.12 (1 H, m), 7.64 (1 H, dd, J 8.2 and 4.1), 7.85 (1 H, m), 8.14 (1 H, d, J 8.4), 8.27 (1 H, s), 8.47 (1 H, d, J 7.8), 9.00 (1 H, dd, J 4.1 and 1.6).

### Description 134 7-[6-Chloro-2-methylthiopyrimidin-4-yl]quinoline

4,6-Dichloro-2-methylthiopyrimidine (0.99 g, 5.09 mmol), Description 11 (0.65 g, 2.54 mmol), [1,1′-Bis(diphenylphosphino)ferrocene]palladium(II) chloride (93 mg, 0.12 mmol) and 2M Na₂CO₃(aq) (2.54 ml) were suspended in 1,4-dioxane (20 ml) and heated to 100° C. for 16 hours under nitrogen. After cooling to room temperature the mixture was filtered through a pad of Celite®, washing the residue ethyl acetate. The filtrate was concentrated under reduced pressure to give a brown residue, which was partitioned between water and ethyl acetate. The aqueous phase was washed with ethyl acetate, the combined organic phases were washed (brine), dried (sodium sulfate) and concentrated to give a dark-brown oil, which was purified by flash chromatography using a Biotage-Horizon® HPFC system (40S cartridge, gradient elution from 0-50% ethyl acetate/isohexane) to give a pale yellow solid (0.47 g, 64%). ¹H NMR (400 MHz, CDCl₃) 2.70 (3 H, s), 7.49 (1 H, dd, J 8.3 and 4.2), 7.57 (1 H, s), 7.95 (1 H, d, J 8.6), 8.22-8.28 (2 H, m), 8.81 (1 H, s), 9.00 (1 H, dd, J 4.2 and 1.7).

### Description 136 6-Chloro-2-trifluoromethyl-N-[4-trifluoromethylphenyl]pyrimidin-4-amine

Prepared from 4,6-dichloro-2-trifluoromethylpyrimidine [U.S. Pat. No. 4,963,678] and 4-trifluoromethylaniline according to the procedure of Description 95 to give a pale yellow oil. ¹H NMR (400 MHz, CDCl₃) 6.86 (1 H, s), 7.25 (1 H, br s), 7.55 (2 H, d, J 8.5), 7.70 (2 H, d, J 8.5); m/z (ES⁺) 342 (M+H⁺).

### Description 137 6-Quinoxalin-6-ylpyrimidin-4-amine

6-Bromoquinoxaline (210 mg, 1.44 mmol), potassium acetate (141 mg, 1.44 mmol), bis(pinacolato)diboron (383 mg, 1.51 mmol) and [1,1′-bis(diphenylphosphino)ferrocene]palladium(II) chloride (52 mg, 0.072 mmol) were suspended in dioxane (10 ml) and heated to 100° C. for 16 hours. 4-Amino-6-chloropyrimidine [WO-A-0245652] (186 mg, 1.44 mmol), [1,1′-bis(diphenylphosphino)ferrocene]-palladium(II) chloride (52 mg, 0.072 mmol) and 2M Na₂CO₃(aq) (2 ml) were added and the mixture was heated at 100° C. for a further 16 hours. The mixture was partitioned between EtOAc and water, the aqueous phase was extracted with EtOAc, the combined organic phases were washed (brine), dried (sodium sulfate) and concentrated under reduced pressure. The residue was triturated with EtOAc to give a white solid, which was used in the next step without purification (170 mg).

### Description 138 6-(6-Chloro-5-methylpyrimidin-4-yl)quinoxaline

Prepared from 6-bromoquinoxaline and 4,6-dichloro-5-methylpyrimidine according to the procedure of Description 137 to give an off-white solid (170 mg, 92%). ¹H NMR (360 MHz, CDCl₃) 2.53 (3 H, s), 8.01 (1 H, d, J 8.7), 8.27 (1 H, d, J 8.7), 8.31 (1 H, s), 8.95 (3 H, m); m/z (ES⁺) 257 (M+H⁺).

### Description 139 5-Methyl-2-trifluoromethylpyrimidine-4,6-diol

To a suspension of sodium hydride (60% dispersion in oil) (52.5 g, 1311 mmol) in anhydrous toluene (400 ml) was added dropwise 1-butanol (118 ml, 1311 mmol) at such a rate so as to maintain the internal temperature at 40° C. After complete addition the mixture was stirred at room temperature overnight. To this mixture was added methyl malonamide (50 g, 430 mmol), followed by ethyl trifluoroacetate (51.2 ml, 430 mmol), and the resulting mixture heated at 100° C. for 3 hours, then stirred at room temperature overnight. The mixture was extracted with water (3×300 ml), the combined water layers acidified to pH=1 with conc. HCl and the resultant precipitate removed by filtration and dried in-vacuo to give the title compound as a white solid (35.5 g, 43%). ¹H NMR (500 MHz, DMSO-d₆) 1.96 (3 H, s), 12.40 (2 H, br s).

### Description 140 4,6-Dichloro-5-methyl-2-trifluoromethylpyrimidine

To a mixture of Description 139 (15.00 g, 77.3 mmol) in phosphorous oxychloride (33.14 ml, 355.58 mmol) was added dropwise triethylamine (21.5 ml, 154.6 mmol). After complete addition the mixture was heated at 100° C. for 3 hours. The mixture was allowed to cool to room temperature and poured with stirring onto ice/water (400 ml). The mixture was extracted with dichloromethane (3×100 ml), the combined dichloromethane layers dried over Na₂SO₄ , filtered through a 2 inch plug of silica (washing with more dichloromethane) and evaporated to give the title compound as a pale orange solid (8.5 g, 48%). ¹H NMR (500 MHz, CDCl₃) 2.58 (3 H, s).

### Description 141 6-Chloro-5-methyl-2-trifluoromethylpyrimidin-4-amine

Prepared from Description 140 according to the procedure of Description 16 to give a white solid (2.5 g, 32%). ¹H NMR (500 MHz, DMSO-d₆) 2.16 (3 H, s), 7.50 (1 H, br s), 8.00 (1 H, br s).

### Description 142 5-Methyl-6-quinoline-7-yl-2-trifluoromethylpyrimidin-4-amine

Prepared from Description 141 and Description 11 according to the procedure of Description 1 (490 mg, 68%). ¹H NMR (400 MHz, DMSO-d₆) 2.17 (3 H, s), 7.62 (1 H, dd, J 8.3 and 4.2), 7.77 (1 H, dd, J 8.4 and 1.6), 8.11 (1 H, d, J 8.4), 8.17 (1 H, d, J 0.6), 8.45 (1 H, d, J 8.3), 8.99 (1 H, dd, J 4.2 and 1.2).

### EXAMPLE 1

### 4-Quinolin-8-yl-N-[4-trifluoromethylphenyl]pyrimidin-2-amine

To a mixture of Description 1 (100 mg, 0.42 mmol) and 4-trifluoromethylaniline (0.052 ml, 0.42 mmol) in anhydrous toluene (15 ml) was added sodium tert-butoxide (60 mg, 0.62 mmol) and 2′-(dimethylamino)-2-biphenylyl palladium(II) chloride dinorbornylphosphine complex [Angew. Chem., 2002, 41, 3668; CAS number 359803-53-5](23 mg, 0.042 mmol), the mixture was de-gassed three times and heated at reflux overnight. The mixture was cooled and diluted with dichloromethane (10 ml) and the resulting mixture loaded directly onto a silica gel chromatography column: (eluent 2% MeOH in DCM+0.5% NH₄OH). The product was further purified by mass-directed HPLC to give the title compound as a white solid (15 mg, 10%). ¹H NMR (500 MHz, CDCl₃) 7.37 (1 H, br s), 7.42 (1 H, dd, J 8.3 and 4.2), 7.50 (1 H, d, J 8.6), 7.64 (1 H, t, J 7.9 and 7.6), 7.68 (1 H, d, J 4.9), 7.77 (1 H, d, J 8.6), 7.90 (1 H, dd, J 8.3 and 1.2), 8.18-8.21 (2 H, m), 8.51 (1 H, d, J 5.2), 8.93 (1 H, dd, J 3.9 and 1.7); m/z (ES⁺) 367 (M+H⁺).

### EXAMPLE 2

### 6-Quinolin-8-yl-N-[4-trifluoromethylphenyl]pyrazin-2-amine

To a mixture of Description 2 (200 mg, 0.83 mmol) and 4-trifluoromethylaniline (0.104 ml, 0.83 mmol) in anhydrous 1,4-dioxane (15 ml) was added cesium carbonate (379 mg, 1.16 mmol), 4,5-bis(diphenylphosphino)-9,9-dimethylxanthene (29 mg, 0.05 mmol), and Pd₂(dba)₃ (15 mg, 0.017 mmol). The mixture was de-gassed three times and heated at reflux overnight. The mixture was cooled, diluted with dichloromethane (10 ml), filtered through hyflo and the filtrate loaded directly onto a silica gel chromatography column: (eluent 2% MeOH in DCM+0.5% NH₄OH). The product was further purified by mass-directed HPLC to give the title compound as a white solid (20 mg, 6.5%). ¹H NMR (500 MHz, CDCl₃) 6.92 (1 H, br s), 7.49 (1 H, dd, J 8.3 and 4.2), 7.56 (2 H, d, J 8.6), 7.68 (2 H, d, J 8.6), 7.71 (1 H, d, J 7.6), 7.95 (1 H, dd, J 8.3 and 1.3), 8.17 (1 H, dd, J 7.4 and 1.5), 8.25-8.27 (2 H, m), 8.95 (1 H, s), 9.00 (1 H, dd, J 4.2 and 1.7); m/z (ES⁺) 367 (M+H⁺).

The following compounds were made by the procedure of Example 2:

### EXAMPLE 3

### 5-Quinolin-8-yl-N-[4-trifluoromethylphenyl]pyridazin-3-amine

Prepared from Description 5 and 4-trifluoromethylaniline to give a white solid (9 mg, 3%). ¹H NMR (500 MHz, DMSO-d₆) 7.57 (1 H, d, J 1.7), 7.66 (1 H, dd, J 8.3 and 4.2), 7.70 (1 H, d, J 8.8), 7.79 (1 H, t, J 8.1 and 7.4), 8.00 (1 H, dd, J 7.1 and 1.3), 8.05 (1 H, d, J 8.3), 8.17 (1 H, d, J 8.3), 8.52 (1 H, dd, J 8.3 and 1.7), 9.00 (1 H, dd, J 4.0 and 1.8), 9.08 (1 H, d, J 1.7), 9.81 (1 H, s); m/z (ES⁺) 367 (M+H⁺).

### EXAMPLE 4

### 6-Quinolin-8-yl-N-[4-trifluoromethylphenyl]pyrimidin-4-amine

Prepared from Description 6 and 4-trifluoromethylaniline to give a white solid (6 mg, 4%). ¹H NMR (500 MHz, CDCl₃) 7.47 (1 H, dd, J 8.3 and 4.2), 7.56 (2 H, d, J 8.8), 7.62 (2 H, d, J 8.8), 7.71 (1 H, t, J 7.9 and 7.6), 7.78 (1 H, s), 7.82 (1 H, s), 7.94 (1 H, dd, J 8.3 and 1.3), 8.26 (1 H, dd, J 8.3 and 1.5), 8.32 (1 H, dd, J 7.4 and 1.3), 8.87 (1 H, s), 8.94 (1 H, dd, J 4.2 and 1.7); m/z (ES⁺) 367 (M+H⁺).

### EXAMPLE 5

### 6-Quinolin-7-yl-N-[4-trifluoromethylphenyl]pyrazin-2-amine

Prepared from Description 12 and 4-trifluoromethylaniline to give a pale yellow solid (70 mg, 7%). ¹H NMR (500 MHz, CDCl₃) 6.87 (1 H, br s), 7.47 (1 H, dd, J 8.3 and 4.2), 7.66 (2 H, d, J 8.6), 7.77 (2 H, d, J 8.6), 7.97 (1 H, d, J 8.6), 8.21-8.26 (3 H, m), 8.74 (2 H, s), 9.00 (1 H, dd, J 4.2 and 1.7); m/z (ES⁺) 367 (M+H⁺).

### EXAMPLE 6

### 4-Quinolin-7-yl-N-[4-trifluoromethylphenyl]pyrimidin-2-amine

Prepared from Description 13 and 4-trifluoromethylaniline to give a white solid (55 mg, 18%). ¹H NMR (500 MHz, CDCl₃) 7.43 (2 H, d, J 5.2), 7.46 (1 H, br s), 7.49 (1 H, dd, J 8.1 and 4.2), 7.63 (2 H, d, J 8.6), 7.88 (2 H, d, J 8.6), 7.97 (1 H, d, J 8.6), 8.23 (1 H, d, J 8.3), 8.31 (1 H, dd, J 8.6 and 1.7), 8.59 (1 H, d, J 5.4), 8.78 (1 H, s), 9.00 (1 H, dd, J 4.2 and 1.7); m/z (ES⁺) 367 (M+H⁺).

### EXAMPLE 7

### 6-Quinolin-7-yl-N-[4-trifluoromethylphenyl]pyrimidin-4-amine

Prepared from Description 14 and 4-trifluoromethylbromobenzene to give a white solid (180 mg, 60%). ¹H NMR (500 MHz, CDCl₃) 7.12 (1 H, br s), 7.35 (1 H, d, J 1.2), 7.47 (1 H, dd, J 8.2 and 4.3), 7.65 (4 H, q, J 8.6), 7.95 (1 H, d, J 8.6), 8.21 (1 H, dd, J 8.6 and 1.2), 8.31 (1 H, dd, J 8.6 and 1.6), 8.63 (1 H, d, J 1.6), 8.91 (1 H, d, J 1.2), 8.98 (1 H, dd, J 4.3 and 2.0); m/z (ES⁺) 367 (M+H⁺).

### EXAMPLE 8

### 5-Quinolin-7-yl-N-[4-trifluoromethylphenyl]pyridazin-3-amine

Prepared from Description 15 and 4-trifluoromethylaniline to give a pale yellow solid (25 mg, 8%). ¹H NMR (500 MHz, DMSO-d₆) 7.60 (1H, d, J 2.0), 7.63 (1 H, dd, J 8.4 and 4.2), 7.70 (2 H, d, J 8.6), 8.04 (3 H, m), 8.20 (1 H, d, J 8.6), 8.47 (1 H, d, J 8.1), 8.49 (1 H, s), 9.01 (1 H, dd, J 4.2 and 1.5), 9.35 (1 H, d, J 2.0), 9.83 (1 H, s); m/z (ES⁺) 367 (M+H⁺).

### EXAMPLE 9

### 6-Quinolin-7-yl-N-[5-trifluoromethylpyridin-2-yl]pyrimidin-4-amine

Prepared from Description 14 and 2-bromo-5-trifluoromethylpyridine to give a white solid (100 mg, 60%). ¹H NMR (500 MHz, CDCl₃) 7.48 (1 H, dd, J 8.3 and 4.2), 7.73 (1 H, d, J 8.8), 7.79 (1 H, br s), 7.90(1 H, dd, J 8.7 and 2.2), 7.97 (1 H, d, J 8.6), 8.23 (1 H, d, J 7.7), 8.31 (1 H, s), 8.36 (1 H, dd, J 8.5 and 1.7), 8.67 (1 H, s), 8.80 (1 H, s), 9.00 (1 H, s), 9.01 (1 H, d, J 1.7); m/z (ES⁺) 368 (M+H⁺).

### EXAMPLE 10

### 6-Quinolin-7-yl-N-[6-trifluoromethylpyridin-3-yl]pyrimidin-4-amine

Prepared from Description 14 and 3-bromo-6-trifluoromethylpyridine to give a white solid (120 mg, 72%). ¹H NMR (500 MHz, DMSO-d₆) 7.57 (1 H, s), 7.62 (1 H, dd, J 8.3 and 4.2), 7.89 (1 H, d, J 8.7), 8.15 (1 H, d, J 8.6), 8.27 (1 H, dd, J 8.6 and 1.7), 8.45 (1 H, d, J 8.1), 8.59 (1 H, dd, J 8.6 and 2.2), 8.69 (1 H, s), 8.91 (1 H, s), 9.00 (2 H, s), 10.38 (1 H, br s); m/z (ES⁺) 368 (M+H⁺).

### EXAMPLE 11

### 5-Methoxy-6-quinolin-7-yl-N-[4-trifluoromethylphenyl]pyrimidin-4-amine

Prepared from Description 17 and 4-trifluoromethylbromobenzene to give a white solid (80 mg, 26%). ¹H NMR (400 MHz, CDCl₃) 3.66 (3 H, s), 7.48 (1 H, dd, J 8.3 and 4.2), 7.64 (1 H, d, J 8.6), 7.67 (1 H, br s), 7.92 (1 H, d, J 8.6), 7.96 (1 H, d, J 8.6), 8.23 (1 H, dd, J 8.6 and 1.6), 8.68 (1 H, s), 8.83 (1 H, s), 8.97 (1 H, d, J 1.6); m/z (ES⁺) 397 (M+H⁺).

### EXAMPLE 12

### 5-Methyl-6-quinolin-7-yl-N-[4-trifluoromethylphenyyl pyrimidin-4-amine

Prepared from Description 19 and 4-trifluoromethylbromobenzene to give a white solid (190 mg, 59%). ¹H NMR (400 MHz, CDCl₃) 2.38 (3 H, s), 6.80 (1 H, br s), 7.47 (1 H, dd, J 8.2 and 4.2), 7.64 (2 H, d, J 8.6), 7.79-7.85 (2 H, m), 7.96 (1 H, d, J 8.4), 8.22 (1 H, d, J 1.5), 8.23 (1 H, d), 8.80 (1 H, s), 8.97 (1 H, d, J 1.7); m/z (ES⁺) 381 (M+H⁺).

### EXAMPLE 13

### 5-Fluoro-6-quinolin-7-yl-N-[4-trifluoromethylphenyl]pyrimidin-4-amine

Prepared from Description 21 and 4-trifluoromethylbromobenzene to give a white solid (194 mg, 61%). ¹H NMR (400 MHz, CDCl₃) 7.27 (1 H, d, J 3.1), 7.49 (1 H, dd, J 8.2 and 4.3), 7.66 (2 H, d, J 8.6), 7.90 (2 H, d, J 8.6), 7.98 (1 H, d, J 8.6), 8.23 (1 H, d, J 8.2), 8.29 (1 H, d, J 8.6), 8.70 (1 H, d, J 1.6), 8.83 (1 H, s), 9.00 (1 H, dd, J 4.3 and 1.6); m/z (ES⁺) 385 (M+H⁺).

### EXAMPLE 14

### 2-Methoxy-6-quinoline-7-yl-N-[4-trifluoromethylphenyl]pyrimidin-4-amine

Prepared from Description 23 and 4-trifluoromethylbromobenzene to give an off white solid (130 mg, 41%). ¹H NMR (400 MHz, CDCl₃) 4.11 (3 H, s), 7.00 (1 H, s), 7.38 (1 H, br s), 7.45 (1 H, dd, J 8.2 and 4.3), 7.64 (4 H, s), 7.90 (1 H, d, J 8.6), 8.19 (1 H, d, J 7.4), 8.27 (1 H, dd, J 8.6 and 1.6), 8.69 (1 H, s), 8.96 (1 H, dd, J 4.3 and 2.0); m/z (ES⁺) 397 (M+H⁺).

### EXAMPLE 15

### 2-Methyl-6-quinolin-7-yl-N-[4-trifluoromethylphenyl]pyrimidin-4-amine

Prepared from Description 25 and 4-trifluoromethylbromobenzene to give an off white solid (190 mg, 59%). ¹H NMR (400 MHz, CDCl₃) 2.72 (3 H, s), 7.01 (1 H, br s), 7.18 (1 H, s), 7.45 (1 H, dd, J 8.2 and 4.3), 7.61 (2 H, d, J 8.6), 7.66 (2 H, d, J 8.6), 7.93 (1 H, d, J 8.2), 8.20 (1 H, d, J 8.2), 8.30 (1 H, dd, J 8.6 and 2.0), 8.63 (1 H, s), 8.97 (1 H, dd, J 4.3 and 2.0); m/z (ES⁺) 381 (M+H⁺).

### EXAMPLE 16

### 6-(3-Methylquinolin-7-yl)-N-[4-trifluoromethylphenyl]pyrimidin-4-amine

Prepared from Description 28 and 4-trifluoromethylbromobenzene to give a white solid (90 mg, 28%). ¹H NMR (360 MHz, CDCl₃) 2.55 (3 H, s), 7.17 (1 H, br s), 7.33 (1 H, s), 7.33 (1 H, s), 7.86 (1 H, d, J 8.4), 7.96 (1 H, s), 8.26 (1 H, dd, J 8.4 and 1.7), 8.59 (1 H, s), 8.82 (1 H, d, J 2.1), 8.89 (1 H, s); m/z (ES⁺) 381 (M+H⁺).

### EXAMPLE 17

### 6-Quinolin-5-yl-N-[4-trifluoromethylphenyl]pyrimidin-4-amine

Prepared from Description 30 and 4-trifluoromethylaniline to give a white solid. ¹H NMR (400 MHz DMSO-d₆) 7.16 (1 H, d, J 1.2), 7.59 (1 H, dd, J 4.2 and 8.8), 7.72 (2 H, d, J 8.8), 7.83 (1 H, dd, J 7.1 and 1.2), 7.88-7.91 (1 H, m), 8.01 (2 H, d, J 8.6), 8.17 (1 H, d, J 8.3), 8.69 (1H, d, J 8.6), 8.90 (1 H, d, J 1.2), 8.98 (1 H, dd, J 3.9 and 1.7), 10.20 (1 H, s); m/z (ES⁺) 367 (M+H⁺).

### EXAMPLE 18

### 6-Quinolin-6-yl-N-[4-trifluoromethylphenyl]pyrimidin-4-amine

Prepared from Description 31 and 4-trifluoromethylbromobenzene to give a white solid. ¹H NMR (400 MHz, DMSO-d₆) 7.52 (1 H, d, J 1.2), 7.63 (1 H, dd, J 8.3 and 4.2), 7.72 (2 H, d, J 8.6), 8.00 (2 H, d, J 8.6), 8.18 (1 H, d, J 8.8), 8.40 (1 H, dd, J 8.8 and 2.2), 8.57 (1 H, dd, J 8.3 and 1.2), 8.75 (1 H, d, J 2.0), 8.88 (1 H, d, J 1.2), 8.99 (1 H, dd, J 4.2 and 1.7), 10.21 (1 H, s); m/z (ES⁺) 367 (M+H⁺).

### EXAMPLE 19

### 6-(2-Methylquinoline-7-yl)-N-[4-trifluoromethylphenyl]pyrimidin-4-amine

Prepared from Description 33 and 4-trifluoromethylbromobenzene to give a white solid. ¹H NMR (400 MHz, DMSO-d₆) 2.70 (3 H, s), 7.49-7.52 (2 H, m), 7.72 (2 H, d, J 8.8), 8.00 (2 H, d, J 8.6), 8.08 (1 H, d, J 8.6), 8.17-8.19 (1 H, m), 8.32 (1 H, d, J 8.6), 8.58 (1 H, s), 8.88 (1 H, d, J 1.0), 10.15 (1 H, s); m/z (ES⁺) 381 (M+H⁺).

### EXAMPLE 20

### 6-(6-Fluoroquinolin-7-yl)-N-[4-trifluoromethylphenyl]pyrirnidin-4-amine

Prepared from Description 39 and 4-trifluoromethylbromobenzene to give a white solid (28 mg, 43%). ¹H NMR (400 MHz, DMSO-d₆) 7.49 (1 H, s), 7.63-7.67 (1 H, m), 7.72 (2 H, d, J 8.4), 8.01 (3 H, m), 8.44 (1 H, dd, J 8.4 and 1.1), 8.75 (1 H, d, J 7.4), 8.91 (1 H, d, J 1.1), 8.99 (1 H, dd, J 4.2 and 1.8), 10.25 (1 H, s); m/z (ES⁺) 385 (M+H⁺).

### EXAMPLE 21

### 6-(8-Fluoroquinolin-7-yl)-N-[4-trifluoromethylphenyl]pyrimidin-4-amine

Prepared from Description 41 and 4-trifluoromethylbromobenzene to give a white solid. ¹H NMR (400 MHz DMSO-d₆) 7.60 (1 H, s), 7.71-7.74 (3 H, m), 7.96 (1H, d, J 8.6), 8.01 (2 H, d, J 8.6), 8.28 (1 H, dd, J 8.6 and 7.0), 8.52 (1 H, d, J 8.6), 8.92 (1 H, d, J 1.2), 9.06 (1 H, dd, J 4.3 and 1.6), 10.27 (1 H, s); m/z (ES⁺) 385 (M+H⁺).

### EXAMPLE 22

### N-[4-(trifluoromethyl)phenyl]-6-[6-trifluoromethylquinolin-7-yl]pyrimidin-4-amine

Prepared from Description 42 and 4-trifluoromethylbromobenzene to give an off white solid (110 mg, 36%). ¹H NMR (400 MHz, CDCl₃) 6.96 (1 H, s), 7.47 (1 H, dd, J 8.4 and 4.3), 7.61 (4 H, s), 7.66 (1 H, br s), 8.21 (1 H, s), 8.30-8.32 (2 H, m), 8.87 (1 H, d, J 1.0), 9.07 (1 H, dd, J 4.3 and 1.7); m/z (ES⁺) 435 (M+H⁺).

### EXAMPLE 23

### 6-(8-Methylquinolin-7-yl)-N-[4-trifluoromethylphenyl]pyrimidin-4-amine

Prepared from Description 44 and 4-trifluoromethylbromobenzene to give a white solid (190 mg, 59%). ¹H NMR (400 MHz, CDCl₃) 2.87 (3 H, s), 6.91 (1 H, d, J 1.1), 7.26 (1 H, br s), 7.46 (1 H, dd, J 8.2 and 4.3), 7.59-7.61 (5 H, m), 7.75 (1 H, d, J 8.2), 8.17 (1 H, dd, J 8.2 and 2.0), 8.91 (1 H, d, J 1.1), 9.00 (1 H, dd, J 4.3 and 2.0); m/z (ES⁺) 381 (M+H⁺).

### EXAMPLE 24

### 5-Fluoro-6-(8-methylquinolin-7-yl)-N-[4-trifluoromethylphenyl]pyrimidin-4-amine

Prepared from Description 45 and 4-trifluoromethylbromobenzene to give a white solid (155 mg, 49%). ¹H NMR (500 MHz, CDCl₃) 2.80 (3 H, s), 7.21 (1 H, br s), 7.49 (1 H, dd, J 8.3 and 4.2), 7.58 (1 H, d, J 8.5), 7.66 (2 H, d, J 8.6), 7.79 (1 H, d, J 8.5), 7.90 (2 H, d, J 8.6), 8.19 (1 H, dd, J 8.3 and 1.7), 8.71 (1 H, d, J 1.6), 9.03 (1 H, dd, J 4.2 and 1.7); m/z (ES⁺) 399 (M+H⁺).

### EXAMPLE 25

### 6-Isoquinolin-7-yl-N-[4-trifluoromethylphenyl]pyrimidin-4-amine

Prepared from Description 46 and 4-trifluoromethylbromobenzene to give a white solid. ¹H NMR (500 MHz DMSO-d₆) 7.52 (1 H, s), 7.72 (2 H, d, J 8.8), 7.93 (1 H, d, J 5.6), 8.00 (2 H, d, J 8.6), 8.15 (1 H, d, J 8.6), 8.43 (1 H, dd, J 8.7 and 1.6), 8.59 (1 H, d, J 5.6), 8.88 (2 H, d, J 8.6), 9.51 (1 H, s), 10.21 (1 H, s); m/z (ES⁺) 367 (M+H⁺).

### EXAMPLE 26

### 6-Quinolin-8-yl-N-[4-trifluoromethylphenyl]pyridazin-4-amine

A mixture of Description 8 (100 mg, 0.38 mmol), quinoline-8-boronic acid (126 mg, 0.73 mmol), 2M sodium carbonate (0.365 ml, 0.73 mmol), and Pd(dppf)Cl₂ (10 mg, 0.011 mmol) was heated at 170° C. for 40 mins in a Smith microwave reactor. The mixture was diluted with dichloromethane (20 ml) and washed with water (2×20 ml), sat. NaCl (15 ml), dried over Na₂SO₄ , filtered and evaporated. The residue was purified by PREP-TLC: (eluent 10% MeOH in DCM+0.5% NH₄OH), followed by mass-directed HPLC to give the title compound as a white solid (9 mg, 6.7%). ¹H NMR (500 MHz, DMSO-d₆) 7.53 (2 H, d, J 8.3), 7.63 (1 H, dd, J 8.4 and 4.2), 7.72 (2 H, d, J 8.3), 7.78 (1 H, t, J 7.8 and 7.4), 7.99 (1 H, d, J 2.7), 8.15 (1 H, d, J 8.3), 8.23 (1 H, dd, J 7.1 and 1.2), 8.51 (1 H, dd, J 8.3 and 1.7), 9.00 (1 H, d, J 2.7), 9.04 (1 H, dd, J 4.2 and 1.7), 9.66 (1 H, s); m/z (ES⁺) 367 (M+H⁺).

### EXAMPLE 27

### 4-Quinolin-8-yl-N-[4-trifluoromethylphenyl]-1,3,5-triazin-2-amine

To a mixture of Description 9 (790 mg, 2.89 mmol), quinoline-8-boronic acid (500 mg, 2.89 mmol) and 2M Sodium carbonate (2.89 ml, 5.78 mmol) in a mixture of toluene (50 ml) and ethanol (10 ml) was added Pd(PPh₃)₄ (171 mg, 0.14 mmol), the mixture degassed three times and heated at reflux overnight. The reaction mixture was cooled and diluted with EtOAc (50 ml), washed with water (2×100 ml), sat NaCl (100 ml), dried over Na₂SO₄, filtered and evaporated. The residue was purified by column chromatography on silica: (eluent 2% MeOH in DCM+0.5% NH₄OH), and product further purified by mass-directed HPLC to give the title compound as a white solid (3.4 mg, 0.32%). ¹H NMR (500 MHz, CDCl₃) 7.51 (1 H, dd, J 8.4 and 4.2), 7.60 (2 H, d, J 8.3), 7.68 (2 H, m), 7.86 (2 H, d, J 8.3), 8.01 (1 H, dd, J 8.1 and 1.2), 8.17 (1 H, m), 8.26 (1 H, dd, J 8.3 and 1.3), 8.99 (1 H, s), 9.02 (1 H, m); m/z (ES⁺) 368 (M+H⁺).

### EXAMPLE 28

### 5-Nitro-6-quinoline-7-yl-N-[4-trifluoromethylphenyl]pyrimidin-4-amine

To a mixture of Description 29 (2.01 g, 6.3 mmol) and Description 11 (2.41 g, 9.45 mmol) in a mixture of toluene (50 ml) and ethanol (10 ml) was added 2M sodium carbonate (3.15 ml, 6.3 mmol) and Pd(PPh₃)₄ (364 mg, 0.315 mmol). The mixture was de-gassed three times and heated at reflux overnight. The cooled mixture was diluted with EtOAc (100 ml) and washed with water (200 ml), sat. NaCl (100 ml), dried over Na₂SO₄, filtered and evaporated. The residue was purified by column chromatography on silica: (eluent 1% MeOH in DCM+0.5% NH₃) then a second column (eluent 20% EtOAc in iso-hexanes). The product triturated with diethyl ether to give the title compound as a pale yellow solid (100 mg, 4%). ¹H NMR (500 MHz, CDCl₃) 7.50 (1 H, dd, J 8.3 and 4.2), 7.70 (2 H, d, J 8.1), 7.72 (1 H, dd, J 8.6 and 1.7), 7.81 (2 H, d, J 8.1), 7.95 (1 H, d, J 8.6), 8.24 (1 H, d, J 7.6), 8.36 (1 H, s), 8.87 (1 H, s), 9.00 (1 H, dd, J 4.2 and 1.7); m/z (ES⁺) 412 (M+H⁺).

### EXAMPLE 29

### 6-Quinolin-7-yl-N⁴-[4-trifluoromethylphenyl]pyrimidine-4,5-diamine

To a nitrogen flushed solution of Example 28 (80 mg, 0.195 mmol) in a mixture of methanol (5 ml) and dichloromethane (5 ml) was added 10% Palladium on carbon (10 mg) and the resulting mixture stirred under a balloon of hydrogen for 2 hours. The catalyst was removed by filtration and the filtrate evaporated. The residue was crystallised from dichloromethane/diethyl ether to give 60 mg (Yield 80%) as an off white solid. ¹H NMR (500 MHz, DMSO-d₆) 5.31 (2 H, br s), 7.60 (1 H, dd, J 8.3 and 4.2), 7.69 (2 H, d, J 8.7), 7.91 (1 H, dd, J 8.4 and 1.6), 8.03 (2 H, d, J 8.7), 8.11 (1 H, d, J 8.4), 8.27 (1 H, s), 8.34 (1 H, s), 8.43 (1 H, d, J 7.5), 8.93 (1 H, s), 8.97 (1 H, dd, J 4.2 and 1.7); m/z (ES⁺) 382 (M+H⁺).

Examples 30-51 were made from the indicated compounds according to the procedure of Example 2.

### EXAMPLE 30

### 6-(8-Fluoroquinolin-7-yl)-5-methyl-N-[5-trifluoromethylpyridin-2-yl]pyrimidin-4-amine

Description 47 and 2-bromo-5-trifluoromethylpyridine gave a solid (171 mg, 72%). ¹H NMR (400 MHz, DMSO-d₆) 2.23 (3 H, d, J 2.2), 7.68 (1 H, dd, J 8.4 and 6.5), 7.73 (1 H, dd, J 8.4 and 4.2), 7.97 (1 H, d, J 8.5), 8.19 (1 H, dd, J 9.0 and 2.4), 8.39 (1 H, d, J 8.8), 8.54 (1 H, d, J 8.4), 8.73 (1 H, s), 8.81 (1 H, s), 9.04 (1 H, dd, J 4.2 and 1.6), 9.66 (1 H, s); m/z (ES⁺) 400 (M+H⁺).

### EXAMPLE 31

### 6-(8-Fluoroquinolin-7-yl)-5-methyl-N-[4-trifluoromethylphenyl]pyrimidin-4-amine

Description 47 and 4-trifluoromethylbromobenzene gave a white solid (22 mg, 14%). ¹H NMR (400 MHz, DMSO-d₆) 2.19 (3 H, d, J 2.2), 7.74-7.66 (4 H, m), 7.96 (1 H, d, J 8.5), 8.03 (2 H, d, J 8.5), 8.54 (1 H, d, J 8.4), 8.68 (1 H, s), 8.99 (1 H, s),9.04 (1 H, dd, J 4.1 and 1.6; m/z (ES⁺) 399 (M+H⁺).

### EXAMPLE 32

### 5-Methoxy-2-methyl-6-quinoline-7-yl-N-[4-trifluoromethylphenyl]pyrimidin-4-amine

Description 51 and 4-trifluoromethylbromobenzene gave a white solid (78 mg, 51%). ¹H NMR (400 MHz, DMSO-d₆) 2.57 (3 H, s), 3.55 (3 H, s), 7.62-7.60 (1 H, dd, J 8.3 and 4.1), 7.71 (2 H, d, J 8.7), 8.24-8.12(4 H, m), 8.45 (1 H, d, J 7.7), 8.68 (1 H, s), 8.99 (1 H, dd, J 4.1 and 1.6), 9.52 (1 H, s); m/z (ES⁺) 411 (M+H⁺).

### EXAMPLE 33

### 2-Methyl-6-(8-methylquinolin-7-yl)-N-[4-trifluoromethylphenyl]pyrimidin-4-amine

Description 52 and 4-trifluoromethylbromobenzene gave a white solid (110 mg, 45%). ¹H NMR (400 MHz, DMSO-d₆) 2.60 (3 H, s), 2.80 (3 H, s), 6.88 (1 H, s), 7.59-7.71 (4 H, m), 7.92 (1 H, d, J 8.4), 8.01 (2 H, d, J 8.3), 8.41 (1 H, d, J 7.9), 9.01 (1 H, d, J 2.3), 10.02 (1 H, s); m/z (ES⁺) 395 (M+H⁺).

### EXAMPLE 34

### N-[2-Fluoro-4-trifluoromethylphenyl]-5-methoxy-6-quinolin-7-ylpyrimidin-4-amine

Description 17 and 1-bromo-2-fluoro-4-trifluoromethylbenzene gave a white solid (80 mg, 48%). ¹H NMR (400 MHz, CDCl₃) 3.68 (3 H, s), 7.43 (1 H, dd, J 9.5 and 1.5), 7.47-7.51 (2 H, m), 7.91 (1 H, J 3.2), 7.97 (1 H, J8.6), 8.21-8.24 (2 H, m), 8.71(1 H, s), 8.85 (1 H, s), 8.94 (1 H, t, J 8.3), 9.00 (1 H, dd, J 4.2 and 1.7); m/z (ES⁺) 415 (M+H⁺).

### EXAMPLE 35

### 5-Methoxy-6-quinolin-7-yl-N-[4-trifluoromethoxyphenyl]pyrimidin-4-amine

Description 17 and 1-bromo-4-trifluoromethoxybenzene gave a white solid (50 mg, 30%). ¹H NMR (400 MHz, CDCl₃) 3.64 (3 H, s), 7.25 (2 H, d, J 9.0), 7.48 (1 H, dd, J 8.1 and 4.2), 7.79 (2 H, d, J 9.0), 7.96 (1 H, d, J 8.6), 8.23 (1 H, m), 8.64 (1 H, s), 8.84 (1 H, s), 9.00 (1 H, dd, J 4.2 and 1.5); m/z (ES⁺) 413 (M+H⁺).

### EXAMPLE 36

### 5-Methyl-6-(8-methylquinolin-7-yl)-N-[4-trifluoromethylphenyl]pyrimidin-4-amine

Description 53 and 4-trifluoromethylbromobenzene gave a white solid (150 mg, 63%). ¹H NMR (400 MHz, CDCl₃) 2.09 (3 H, s), 2.65 (3 H, s), 6.69 (1 H, br s), 7.42 (1 H, d, J 8.4), 7.47 (1 H, dd, J 8.2 and 4.2), 7.65 (2 H, d, J 8.6), 7.78 (1 H, d, J 8.4), 7.85 (2 H, d, J 8.6), 8.19 (1 H, dd, J 8.2 and 1.8), 8.80(1 H, s), 9.01 (1 H, dd, J 4.2 and 1.8); m/z (ES⁺) 395 (M+H⁺).

### EXAMPLE 37

### 5-Methyl-6-(8-methylquinolin-7-yl)-N-[5-trifluoromethylpyridin-2-yl]pyrimidin-4-amine

Description 53 and 2-bromo-5-trifluoromethylpyridine gave a white solid (150 mg, 63%). ¹H NMR (500 MHz, CDCl₃) 2.14 (3 H, s), 2.64 (3 H, s), 7.41 (1 H, d, J 8.4), 7.48 (1 H, dd, J 8.3 and 4.2), 7.62 (1 H, s), 7.78 (1 H, d, J 8.3), 7.98 (1 H, dd, J 8.9 and 2.0), 8.20 (1 H, dd, J 8.2 and 1.6), 8.56 (1 H, s), 8.83 (1 H, d, J 8.8), 8.87 (1 H, s), 9.02 (1 H, dd, J 4.2 and 1.6); m/z (ES⁺) 396 (M+H⁺).

### EXAMPLE 38

### 6-Quinolin-7-yl-5-trifluoromethyl-N-[4-trifluoromethylphenyl]pyrimidin-4-amine

Description 57 and 4-trifluoromethylbromobenzene gave a white solid (28 mg, 19%). ¹H NMR (400 MHz, CDCl₃) 7.41 (1 H, s), 7.47 (1 H, dd, J 8.1 and 4.3), 7.66 (2 H, d, J 8.4), 7.75 (1 H, d, J 8.4), 7.92 (1 H, d, J 8.6), 8.21 (1 H, d, J 8.4), 8.85 (1 H, s), 8.98 (1 H, dd, J 4.3 and 1.5); m/z (ES⁺) 435 (M+H⁺).

### EXAMPLE 39

### 5-Ethyl-6-quinolin-7-yl-N-[4-trifluoromethylphenyl]pyrimidin-4-amine

Description 59 and 4-trifluoromethylbromobenzene gave a white solid (250 mg, 77%). ¹H NMR (400 MHz, DMSO-d₆) 1.09 (3 H, t, J 7.4), 2.79 (2 H, q, J 7.4), 7.62 (1 H, dd, J 8.3 and 4.2), 7.71 (3 H, d, J 8.5), 8.01 (2 H, d, J 8.5), 8.09 (1 H, s), 8.11 (1 H, d, J 8.4), 8.46 (1 H, d, J 8.3), 8.63 (1 H, s), 8.94 (1 H, s), 8.98 (1 H, dd, J 4.2 and 1.7); m/z (ES⁺) 395 (M+H⁺).

### EXAMPLE 40

### 5-Ethyl-6-quinolin-7-yl-N-[5-trifluoromethylpyridin-2-yl]pyrimidin-4-amine

Description 59 and 2-bromo-5-trifluoromethylpyridine gave a white solid (175 mg, 55%). ¹H NMR (500 MHz, CDCl₃) 1.34 (3 H, t, J 7.6), 2.80 (2 H, d, J 7.6), 7.48 (1 H, dd, J 8.3 and 4.2), 7.69-7.72 (2 H, m), 7.95-7.98 (2 H, m), 8.21 (1 H, s), 8.23 (1 H, d, J 8.0), 8.55 (1 H, s), 8.81 (1 H, d, J 8.8), 8.86 (1 H, s), 8.99 (1 H, dd, J 4.2 and 1.7); m/z (ES⁺) 396 (M+H⁺).

### EXAMPLE 41

### 5-Methyl-6-quinolin-7-yl-N-[5-trifluoromethylpyridin-2-yl]pyrimidin-4-amine

Description 19 and 2-bromo-5-trifluoromethylpyridine gave a white solid (180 mg, 55%). ¹H NMR (500 MHz, CDCl₃) 2.43 (3 H, s), 7.48 (1 H, dd, J 8.3 and 4.2), 7.66 (1 H, s), 7.80 (1 H, dd, J 8.4 and 1.5), 7.97 (2 H, d, J 8.5), 8.23 (2H, m), 8.56 (1 H, s), 8.80 (1 H, d, J 8.8), 8.87 (1 H, s), 9.00 (1 H, dd, J 4.2 and 1.5); m/z (ES⁺) 382 (M+H⁺).

### EXAMPLE 42

### 2-Cyclopropyl-5-methyl-6-quinolin-7-yl-N-[5-trifluoromethylpyridin-2-yl]pyrimidin-4-amine

Description 61 and 2-bromo-5-trifluoromethylpyridine gave a white solid (120 mg, 40%). ¹H NMR (500 MHz, CDCl₃) 1.08 (2 H, m), 1.19 (2 H, m), 2.26 (1 H, m), 2.34 (3 H, m), 7.46 (1 H, dd, J 8.2 and 4.2), 7.58 (1 H, s), 7.78 (1 H, d, J 8.3), 7.93-7.97 (2 H, m), 8.21 (2 H, m), 8.54 (1 H, s), 8.71 (1 H, d, J 8.8), 8.98 (1 H, d, J 2.9); m/z (ES⁺) 422 (M+H⁺).

### EXAMPLE 43

### 2-Cyclopropyl-5-methyl-6-quinolin-7-yl-N-[4-trifluoromethylphenyl]pyrimidin-4-amine

Description 61 and 4-trifluoromethylbromobenzene gave a white solid (150 mg, 50%). ¹H NMR (500 MHz, CDCl₃) 1.01-1.04 (2 H, m), 1.14-1.17 (2 H, m), 2.20-2.23 (1 H, m), 2.29 (3 H, s), 6.69 (1 H, s), 7.45 (1 H, dd, J 8.2 and 4.2), 7.62 (2 H, d, J 8.6), 7.78 (1 H, dd, J 8.3 and 1.3), 7.83 (2 H, d, J 8.6), 7.93 (1 H, d, J 8.4), 8.21 (2 H, m), 8.97 (1 H, s); m/z (ES⁺) 423 (M+H⁺).

### EXAMPLE 44

### 5-Isopropyl-6-quinolin-7-yl-N-[5-trifluoromethylpyridin-2-yl]pyrimidin-4-amine

Description 63 and 2-bromo-5-trifluoromethylpyridine gave a white solid (120 mg, 40%). ¹H NMR (500 MHz, DMSO-d₆) 1.30 (6 H, d, J 7.3), 3.36 (1 H, quintet, J 7.3), 7.63 (1 H, dd, J 8.3 and 4.2), 7.69 (1 H, dd, J 8.4 and 1.4), 8.05 (1 H, s), 8.12 (1 H, d, J 8.4), 8.22 (1 H, dd, J 8.8 and 1.8), 8.41 (1 H, d, J 8.6), 8.47 (2 H, m), 8.72 (1 H, s), 8.80 (1 H, s), 9.20 (1 H, dd, J 4.2 and 1.0); m/z (ES⁺) 410 (M+H⁺).

### EXAMPLE 45

### 6-(6-Fluoroquinolin-7-yl)-5-methyl-N-[5-trifluoromethylpyridin-2-yl]pyrimidin-4-amine

Description 64 and 2-bromo-5-trifluoromethylpyridine gave a solid (5 mg, 3%). ¹H NMR (500 MHz, DMSO-d₆) 2.22 (3 H, s), 7.66 (1 H, dd, J 8.4 and 4.2), 7.99 (1 H, d, J 10.3), 8.13 (1 H, d, J 7.0), 8.19 (1 H, d, J 8.9), 8.38(1 H, d, J 8.9), 8.46 (1 H, d, J 8.2), 8.73 (1 H, s), 8.80 (1 H, s), 8.97 (1 H, d, J 2.8), 8.97 (1 H, d, J 2.8), 9.66 (1 H, s); m/z (ES⁺) 400 (M+H⁺).

### EXAMPLE 46

### 6-(6-Fluoroquinolin-7-yl)-5-methyl-N-[4-trifluoromethylphenyl]pyrimidin-4-amine

Description 64 and 4-trifluoromethylbromobenzene gave a solid (6 mg, 4%). ¹H NMR (500 MHz, DMSO-d₆) 2.18 (3 H, s), 7.65 (1 H, dd, J 8.2 and 4.0), 7.72 (2 H, d, J 8.6), 7.98 (1 H, d, J 10.2), 8.02 (2 H, d, J 8.4), 8.12 (1 H, d, J 7.0), 8.46 (1 H, d, J 8.1), 8.67 (1 H, s), 8.97 (2 H, m); m/z (ES⁺) 399 (M+H⁺).

### EXAMPLE 47

### 5-Fluoro-6-(8-methylquinolin-7-yl)-N-[5-trifluoromethylpyridin-2-yl]pyrimidin-4-amine

Description 45 and 2-bromo-5-trifluoromethylpyridine gave a white solid (40 mg, 41%). ¹H NMR (500 MHz, CDCl₃) 2.80 (3 H, s), 7.26 (1 H, s), 7.49 (1 H, dd, J 8.3 and 4.2), 7.58 (1 H, J 8.6), 7.80 (1 H, d, J 8.6), 8.00 (1 H, J 8.6), 8.07 (1 H, s), 8.19 (1 H, J 7.6), 8.59 (1 H, s), 8.78 (2 H, t, J 4.5 and 3.9), 9.02 (1 H, d, J 2.7); m/z (ES⁺) 400 (M+H⁺).

### EXAMPLE 48

### N-(2-Quinolin-7-ylpyridin-4-yl)-5-trifluoromethylpyridin-2-amine

Description 65 and 2-bromo-5-trifluoromethylpyridine gave a pale yellow solid (250 mg, 76%). ¹H NMR (400 MHz, CDCl₃) 6.93 (1 H, d, J 8.7), 7.41-7.44 (1 H, m), 7.55 (1 H, dd, J 5.6 and 2.2), 7.74 (1 H, dd, J 8.7 and 2.0), 8.83 (1 H, s), 7.90 (1 H, d, J 8.6), 8.07 (1 H, d, J 1.8), 8.19 (1 H, d, J 8.2), 8.30(1 H, dd, J 8.6 and 1.6), 8.58 (3 H, m), 8.96 (1 H, d, J 1.6); m/z (ES⁺) 367 (M+H⁺).

### EXAMPLE 49

### 2-Quinolin-7-yl-N-[4-trifluoromethylphenyl]pyridin-4-amine

Description 65 and 4-trifluoromethylbromobenzene gave a white solid (170 mg, 52%). ¹H NMR (400 MHz, DMSO-d₆) 7.13 (1 H, dd, J 5.5 and 1.7), 7.46 (2 H, d, J 8.4), 7.57 (1 H, dd, J 8.2 and 4.2), 7.46 (2 H, d, J 8.4), 7.75 (1 H, d, J 1.2), 8.10(1 H, d, J 8.6) 8.29 (1 H, dd, J 8.4 and 1.0), 8.41(1 H, d, J 8.1), 8.50 (1 H, d, J 5.5), 8.61 (1 H, s), 8.97 (1 H, s), 9.41 (1 H, s); m/z (ES⁺) 366 (M+H⁺).

### EXAMPLE 50

### 5-Chloro-6-quinolin-7-yl-N-[4-trifluoromethylphenyl]pyrimidin-4-amine

Description 67 and 4-trifluoromethylbromobenzene gave a white solid (180 mg, 57%). ¹H NMR (400 MHz, DMSO-d₆) 7.65 (1 H, t, J 3.7), 7.74 (2 H, d, J 7.9), 7.94 (1 H, d, J 8.2), 8.01 (2 H, d, J 7.9), 8.13 (1 H, d, J 8.2), 8.41 (1 H, s), 8.47 (1 H, d, J 8.0), 8.72 (1 H, s), 9.01 (1 H, s), 9.58 (1 H, s); m/z (ES⁺) 401 (M+H⁺).

### EXAMPLE 51

### 5-Chloro-6-quinolin-7-yl-N-[5-trifluoromethylpyridin-2-yl]pyrimidin-4-amine

Description 67 and 2-bromo-5-trifluoromethylpyridine gave a white solid (210 mg, 67%). ¹H NMR (400 MHz, DMSO-d₆) 7.64 (1 H, dd, J 8.3 and 4.2), 7.95 (1 H, d, J 8.4), 8.15 (1 H, d, J 8.5), 8.27 (1 H, d, J 8.8), 8.39 (1 H, d, J 8.8), 8.42 (1 H, s), 8.87 (1 H, d, J 8.3), 8.78 (1 H, s), 8.87 (1 H, s), 9.01 (l H, dd, J 4.2 and 1.0), 9.62 (1 H, s); m/z (ES⁺) 402 (M+H⁺).

### EXAMPLE 52

### 7-(5-Methyl-6-{5-trifluoromethylpyridin-2-ylamino}pyrimidin-4-yl)quinolinium benzenesulfonate

To a solution of Example 41 in DMF (40 ml) was added benzenesulfonic acid (1.05 eq., 4.3 g, 27.2 mmol) at 40° C. Isopropyl acetate (10 ml) was added into the solution, which was then seeded with the product (10 mg). The solution was aged for 30 min, then more isopropyl acetate (70 ml) was added over 1˜2 hours, keeping the internal temperature at ca. 40° C. After addition, the batch was cooled to 20-25° C., aged for 2 hours, then filtered. The resulting cake was washed with isopropyl acetate (10 mL), then dried to give the title compound (13.4 g, 95%). 1H NMR (400 MHz, DMSO-d6): δ 10.07 (1H, br s), 9.26 (1H, dd, J=4.8,1.5 Hz), 8.94 (1H, d, J=8.2 Hz), 8.90 (1H, s), 8.79 (1H, m), 8.38 (1H, d, J=8.6 Hz), 8.36 (1H, m), 8.32 (1H, d, J=8.8 Hz), 8.25 (1H, dd, J=8.9, 2.4 Hz), 8.02 (1H, dd, J=8.5, 1.6 Hz), 7.97 (1H, dd, J=8.4, 4.8 Hz), 7.64-7.58 (2 H, om), 7.35-7.26 (3H, om), 2.37 (3H, s); m/z (ES⁺) 382 (M+H⁺).

The following compounds can be prepared according to the procedure described in Example 52.

| Example | Title |
|---|---|
| 53 | 7-(2-Cyclopropyl-5-methyl-6-{5-trifluoromethylpyridin-2- |
| | ylamino}pyrimidin-4-yl)quinolinium benzenesulfonate. |
| 54 | 7-(2-Cyclopropyl-5-methyl-6-{4-trifluoromethylphenyl- |
| | amino}pyrimidin-4-yl)quinolinium benzenesulfonate. |
| 55 | 7-(5-Isopropyl-6-{5-trifluoromethylpyridin-2-ylamino} |
| | pyrimidin-4-yl)quinolinium benzenesulfonate. |
| 56 | 6-Fluoro-7-(5-methyl-6-{5-trifluoromethylpyridin-2- |
| | ylamino}pyrimidin-4-yl)quinolinium benzenesulfonate. |
| 57 | 6-Fluoro-7-(5-methyl-6-{4-trifluoromethylphenylamino} |
| | pyrimidin-4-yl)quinolinium benzenesulfonate. |
| 58 | 7-(5-Fluoro-6-{5-trifluoromethylpyridin-2-ylamino} |
| | pyrimidin-4-yl)-8-methylquinolinium benzenesulfonate. |

Examples 59-79 were made from the indicated compounds according to the procedure of Example 2.

### EXAMPLE 59

### 5-tert-Butyl-6-quinolin-7-yl-N-[4-trifluoromethylphenyl]pyrimidin-4-amine

Description 69 and 4-trifluoromethylbromobenzene gave a white solid (150 mg, 47%). ¹H NMR (500 MHz, DMSO-d₆) 1.29 (9 H, s), 7.60 (1 H, dd, J 8.3 and 4.2), 7.69 (3 H, d, J 8.7), 7.80 (2 H, d, J 8.6), 7.93 (1 H, s), 8.07 (1 H, d, J 8.4), 8.13 (1 H, s), 8.44 (1 H, d, J 8.1), 8.47 (1 H, s), 8.97 (1 H, dd, J4.2 and 1.7); m/z (ES⁺) 423 (M+H⁺).

### EXAMPLE 60

### 5-tert-Butyl-6-quinolin-7-yl-N-[5-trifluoromethylpyridin-2-yl]pyrimidin-4-amine

Description 69 and 2-bromo-5-trifluoromethylpyridine gave a white solid (190 mg, 62%). ¹H NMR (500 MHz, DMSO-d₆) 1.29 (9 H, s), 7.61 (1 H, dd, J 8.3 and 4.2), 7.71 (1 H, d, J 8.3), 7.96 (1 H, s), 8.09 (1 H, d, J 8.3), 8.15 (2 H, m), 8.45 (1 H, d, J 8.1), 8.64 (1 H, s), 8.69 (1 H, s), 8.70 (1 H, s), 8.97 (1 H, d, J 2.7); m/z (ES⁺) 424 (M+H⁺).

### EXAMPLE 61

### 6-(8-Ethylquinolin-7-yl)-N-[4-trifluoromethylphenyl]pyrimidin-4-amine

Description 70 and 4-trifluoromethylbromobenzene gave a white solid (50 mg, 42%). ¹H NMR (500 MHz, DMSO-d₆) 1.25 (3 H, t, J 7.3), 3.32 (2 H, t, J 7.3), 7.04 (1 H, s), 7.58 (1 H, d, J 8.6), 7.60 (1 H, dd, J 8.3 and 4.2), 7.71 (2 H, d, J 8.6), 7.93 (1 H, d, J 8.5), 8.00 (2 H, d, J 8.6), 8.41 (1 H, dd, J 8.3 and 1.5), 8.86 (1 H, s), 9.01 (1 H, dd, J 4.2 and 1.7), 10.13 (1 H, s); m/z (ES⁺) 395 (M+H⁺).

### EXAMPLE 62

### 6-(8-Ethylquinolin-7-yl)-N-[5-trifluoromethylpyridin-2-yl]pyrimidin-4-amine

Description 70 and 2-chloro-5-trifluoromethylpyridine gave a white solid (70 mg, 30%). ¹H NMR (500 MHz, DMSO-d₆) 1.28 (3 H, t, J 7.3), 3.30 (2 H, q, J 7.3), 7.59-7.63 (2 H, m), 7.94 (1 H, d, J 8.5), 7.98 (1 H, d, J 8.8), 8.06 (1 H, s), 8.15 (1 H, dd, J 8.8 and 2.0), 8.42 (1 H, dd, J 8.2 and 1.2), 8.65 (1 H, s), 8.95 (1 H, s), 9.02 (1 H, dd, J 3.9 and 1.5), 10.88 (1 H, s); m/z (ES⁺) 396 (M+H⁺).

### EXAMPLE 63

### 6-(8-Ethylquinolin-7-yl)-5-methyl-N-[4-trifluoromethylphenyl]pyrimidin-4-amine

Description 71 and 4-trifluoromethylbromobenzene gave a white solid (60 mg, 25%). ¹H NMR (500 MHz, DMSO-d₆) 1.09 (3 H, m), 2.05 (3 H, s), 2.87 and 3.25 (2 H, m), 7.39 (1 H, d, J 8.3), 7.60 (1 H, dd, J 8.3 and 4.2), 7.71 (2 H, d, J 8.7), 7.91 (1 H, d, J 8.4), 8.04 (2 H, d, J 8.7), 8.41(1 H, dd, J 8.3 and 1.6), 8.66 (1 H, s), 8.90 (1 H, s), 9.01 (1 H, dd, J 4.2 and 1.6); m/z (ES⁺) 409 (M+H⁺).

### EXAMPLE 64

### 6-(8-Ethylquinolin-7-yl)-5-methyl-N-[5-trifluoromethylpyridin-2-yl]pyrimidin-4-amine

Description 71 and 2-chloro-5-trifluoromethylpyridine gave a white solid (70 mg, 30%). ¹H NMR (500 MHz, DMSO-d₆) 1.09 (3 H, t, J 7.4), 2.09 (3 H, s), 2.86 and 3.25 (2 H, m), 7.39 (1 H, d, J 8.3), 7.61 (1 H, dd, J 8.3 and 4.2), 7.92 (1 H, d, J 8.4), 8.19 (1 H, dd, J 8.9 and 2.2), 8.41-8.44 (2 H, m), 8.72 (1 H, s), 8.80 (1 H, s), 9.01 (1 H, dd, J 4.2 and 1.7), 9.50 (1 H, br s); m/z (ES⁺) 410 (M+H⁺).

### EXAMPLE 65

### N-[2-Fluoro-4-trifluoromethylphenyl]-5-methyl-6-quinolin-7-ylpyrimidin-4-amine

Description 19 and 1-bromo-2-fluoro-4-trifluoromethylbenzene gave a white solid (45 mg, 17%). ¹H NMR (500 MHz, CDCl₃) 2.41 (3 H, s), 7.02 (1 H, d, J 3.7), 7.43 (1 H, d, J 11.2), 7.47-7.51 (2 H, m), 7.80 (1 H, dd, J 8.4 and 1.3), 7.96 (1 H, d, J 8.4), 8.23 (2 H, m), 8.84 (1 H, s), 8.86 (1 H, t, J 8.3), 8.99 (1 H, dd, J 8.2 and 1.2); m/z (ES⁺) 399 (M+H⁺).

### EXAMPLE 66

### 6-(8-Methylquinolin-7-yl)-2-trifluoromethyl-N-[4-trifluoromethylphenyl]pyrimidin-4-amine

Description 77 and 4-trifluoromethylbromobenzene gave a white solid (105 mg, 71%). ¹H NMR (500 MHz, CDCl₃) 2.90 (3 H, s), 7.02 (1 H, s), 7.24 (1 H, s), 7.47 (1 H, dd, J 8.3 and 4.2), 7.64 (2 H, d, J 8.7), 7.68 (2 H, d, J 8.7), 7.77 (1 H, d, J 8.5), 8.18 (1 H, dd, J 8.2 and 1.6), 9.02 (1 H, d, J 1.7); m/z (ES⁺) 449 (M+H⁺).

### EXAMPLE 67

### 6-(8-Methylquinolin-7-yl)-2-trifluoromethyl-N-[5-trifluoromethylpyridin-2-yl]pyrimidin-4-amine

Description 77 and 2-bromo-5-trifluoromethylpyridine gave a white solid (90 mg, 60%). ¹H NMR (500 MHz, CDCl₃) 2.94 (3 H, s), 7.48 (1 H, dd, J 8.2 and 4.2), 7.69 (2 H, m), 7.79 (1 H, d, J 8.5), 7.95 (1 H, dd, J 8.6 and 1.9), 8.08 (2 H, d, J 9.6), 8.19 (1 H, dd, J 8.2 and 1.3), 8.59 (1 H, s), 9.03 (1 H, d, J 1.4); m/z (ES⁺) 450 (M+H⁺).

### EXAMPLE 68

### 2-Methoxymethyl-5-methyl-6-quinolin-7-yl-N-[5-trifluoromethylpyridin-2-yl]pyrimidin-4-amine

Description 81 and 2-chloro-5-trifluoromethylpyridine gave a yellow solid (3.1 g, 68%). ¹H NMR (500 MHz, CDCl₃) 2.40 (3 H, s), 3.59 (3 H, s), 4.69 (2 H, s), 7.47 (1 H, dd, J 8.3 and 4.2), 7.70 (1 H, s), 7.78 (1 H, dd, J 8.4 and 1.3), 7.95 (1 H, d, J 8.4), 7.99 (1 H, dd, J 8.8 and 1.8), 8.23 (2 H, m), 8.55 (1 H, s), 8.89 (1 H, dd, J 4.2 and 1.8), 8.99 (1 H, d, J 1.4); m/z (ES⁺) 426 (M+H⁺).

### EXAMPLE 69

### 5-Fluoro-6-(8-fluoroquinolin-7-yl)-N-[4-trifluoromethylphenyl]pyrimidin-4-amine

Description 82 and 4-trifluoromethylbromobenzene gave a white solid (210 mg, 45%). ¹H NMR (500 MHz, DMSO-d₆) 7.74-7.78 (3 H, m), 7.84 (1 H, dd, J 8.4 and 6.4), 8.01 (1 H, d, J 8.6), 8.12 (2 H, d, J 8.6), 8.55 (1 H, d, J 8.4), 8.70 (1 H, d, J 1.5), 9.07 (1 H, m), 10.19 (1 H, s); m/z (ES⁺) 403 (M+H⁺).

### EXAMPLE 70

### 5-Fluoro-6-(8-fluoroquinolin-7-yl)-N-[5-trifluoromethylpyridin-2-yl]pyrimidin-4-amine

Description 82 and 2-chloro-5-trifluoromethylpyridine gave a yellow solid (160 mg, 34%). ¹H NMR (400 MHz, CDCl₃) 7.58 (1 H, dd, J 8.3 and 4.2), 7.77 (1 H, d, J 8.7), 7.84 (1 H, dd, J 8.6 and 6.0), 8.01 (1 H, dd, J 8.8 and 2.2), 8.12 (1 H, s), 8.26 (1 H, d, J 8.4), 8.60 (1 H, s), 8.77 (2 H, m), 9.07 (1 H, dd, J 4.2 and 1.5); m/z (ES⁺) 404 (M+H⁺).

### EXAMPLE 71

### N-(5-Methyl-6-quinolin-7-ylpyrimidin-4-yl)-6-trifluoromethylpyridazin-3-amine

Description 19 and Description 87 gave an off white solid (190 mg, 45%). ¹H NMR (500 MHz, CDCl₃) 2.50 (3 H, s), 7.50 (1 H, dd, J 8.2 and 4.2), 7.80 (1 H, dd, J 8.4 and 1.5), 7.84 (1 H, d, J 9.4), 7.98 (1 H, d, J 8.4), 8.26 (3 H, m), 8.87 (1 H, s), 8.99 (1 H, dd, J 4.2 and 1.5), 9.10 (1 H, d, J 9.4); m/z (ES⁺) 383 (M+H⁺).

### EXAMPLE 72

### 6-(1,8-Naphthyridin-2-yl)-N-[4-trifluoromethylphenyl]pyrimidin-4-amine

Description 94 and 4-trifluoromethylbromobenzene gave a white solid (80 mg, 48%). ¹H NMR (500 MHz, CDCl₃) 7.24 (1 H, s), 7.55 (1 H, dd, J 8.1 and 4.2), 7.63 (2 H, d, J 8.6), 7.70 (2 H, d, J 8.6), 8.22 (1 H, s), 8.27 (1 H, dd, J 8.1 and 1.7), 8.38 (1 H, d, J 8.6), 8.71 (1 H, d, J 8.3), 8.91 (1 H, s), 9.18 (1 H, d, J 2.2); m/z (ES⁺) 368 (M+H⁺).

### EXAMPLE 73

### 5-Methyl-6-quinolin-8-yl-N-[4-trifluoromethylphenyl]pyrimidin-4-amine

Description 100 and 4-trifluoromethylbromobenzene gave a white solid (80 mg, 48%). ¹H NMR (400 MHz, DMSO-d₆) 1.90 (3 H, s), 7.58 (1 H, dd, J 8.3 and 4.2), 7.70 (2 H, d, J 8.6), 7.73-7.75 (2 H, m), 8.03 (2 H, d, J 8.6), 8.11 (2 H, dd, J 6.1 and 3.5), 8.46 (1 H, dd, J 8.3 and 1.6), 8.63 (1 H, s), 8.85 (1 H, s), 8.87 (1 H, dd, J 4.3 and 1.6); m/z (ES⁺) 381 (M+H⁺).

### EXAMPLE 74

### 5-Methyl-6-quinolin-8-yl-N-[5-trifluoromethylpyridin-2-yl]pyrimidin-4-amine

Description 100 and 2-chloro-5-trifluoromethylpyridine gave an off white solid (180 mg, 75%). ¹H NMR (400 MHz, DMSO-d₆) 1.94 (3 H, s), 7.90 (1 H, dd, J 8.3 and 4.2), 7.72-7.75 (2 H, m), 8.10-8.14 (1 H, m), 8.17 (1 H, dd, J 8.9 and 2.2), 8.40 (1 H, d, J8.8), 8.48 (1 H, dd, J 8.3 and 1.5), 8.70 (1 H, s), 8.76 (1 H, s), 8.85 (1 H, m), 9.50 (1 H, s); m/z (ES⁺) 382 (M+H⁺).

### EXAMPLE 75

### N-[4-Trifluoromethylphenyl]-6-[4-trifluoromethylquinolin-7-yl]pyrimidin-4-amine

Description 120 and 4-trifluoromethylbromobenzene gave a white solid (80 mg, 48%). ¹H NMR (360 MHz, DMSO-d₆) 7.60 (1 H, s), 7.72 (2 H, d, J 8.7), 8.01 (2 H, d, J 8.5), 8.05 (1H, d, J 4.4), 8.29 (1 H, d, J 9.5), 8.48 (1 H, d, J9.1), 8.86 (1 H, s), 8.92 (1 H, s), 9.23 (1 H, d, J 4.1), 10.28 (1 H, s); m/z (ES⁺) 435 (M+H⁺).

### EXAMPLE 76

### 5-Methyl-N-[4-trifluoromethylphenyl]-6-[4-trifluoromethylquinolin-7-yl]pyrimidin-4-amine

Description 121 and 4-trifluoromethylbromobenzene gave a white solid (80 mg, 48%). ¹H NMR (500 MHz, DMSO-d₆) 2.35 (3 H, s), 7.72 (2 H, d, J 8.5), 8.02-8.06 (4 H, m), 8.24 (1 H, d, J 7.1), 8.39 (1 H, s), 8.68 (1 H, s), 8.99 (1 H, s), 9.21 (1 H, d, J 4.3); m/z (ES⁺) 449 (M+H⁺).

### EXAMPLE 77

### 5-Methyl-N-[5-trifluoromethylpyridin-2-yl]-6-[4-trifluoromethylquinolin-7-yl]pyrimidin-4-amine

Description 121 and 2-chloro-5-trifluoromethylpyridine gave a white solid (80 mg, 48%). ¹H NMR (500 MHz, DMSO-d₆) 2.39 (3 H, s), 8.06 (2 H, m), 8.19 (1 H, dd, J 8.8 and 2.2), 8.25 (1 H, d, J 7.1), 8.37(1 H, d, J 9.0), 8.39 (1 H, s), 8.73 (1 H, s), 8.82 (1 H. s), 9.22 (1 H, d, J 4.3); m/z (ES⁺) 450 (M+H⁺).

### EXAMPLE 78

### 6-Quinolin-7-yl-N⁴-[5-trifluoromethylpyridin-2-yl]pyrimidine-4,5-diamine

Description 114 and 5-trifluoromethylpyridin-2-amine gave a solid. ¹H NMR (500 MHz, DMSO-d₆) 5.52 (2 H, s), 7.60 (1 H, dd, J 8.2 and 4.1), 7.92 (1 H, d, J 8.4), 8.11 (1 H, d, J 8.4), 8.15 (1 H, d, J 8.7), 8.35 (2 H, s), 8.44 (1 H, d, J 7.9), 8.49 (1 H, d, J 8.8), 8.70 (1 H, s), 8.97 (1 H, d, J 2.8), 9.69 (1 H, s); m/z (ES⁺) 383 (M+H⁺).

### EXAMPLE 79

### N-[3-Fluoro-5-trifluoromethylpyridin-2-yl]-5-methyl-6-quinolin-7-ylpyrimidin-4-amine

Description 119 and Description 118 gave a solid (12 mg, 4%). ¹H NMR (500 MHz, DMSO-d₆) 2.35 (3 H, s), 7.63 (1 H, dd, J 8.2 and 4.2), 7.85 (1 H, d, J 8.4), 8.13 (1 H, d, J 8.4), 8.21(1 H, s), 8.30 (1 H, d, J 9.2), 8.47 (1 H, d, J 8.4), 8.66 (2 H, d, J 11.9), 8.99 (1 H, s), 9.97 (1 H, s); m/z (ES⁺) 400 (M+H⁺).

### EXAMPLE 80

### (5-Methyl-4-quinolin-7-yl-6-{5-trifluoromethylpyridin-2-ylamino}pyrimidin-2-yl)methanol

A mixture of Example 68 (0.5 g, 1.18 mmol) and 48% aqueous HBr (5 ml) was heated at 90° C. overnight. The cooled mixture was poured onto ice/water and carefully basified by the addition of ammonium hydroxide. The mixture was extracted with dichloromethane (×3) and the combined dichloromethane layers were dried over Na₂SO₄, filtered and evaporated. The residue was purified by column chromatography on silica (eluent: 4% [2M NH₃ in MeOH] in DCM) to give the title compound as a yellow solid (160 mg, 32%). ¹H NMR (500 MHz, DMSO-d₆) 3.31 (3 H, s), 4.58 (2 H, d, J 6.1), 5.20 (1 H, t, J 6.1), 7.62 (1 H, dd, J 7.9 and 3.9), 7.81 (1 H, d, J 8.3), 8.13 (2 H, m), 8.18 (1 H, s), 8.46 (1 H, d, J 8.1), 8.56 (1 H, d, J 8.7), 8.71 (1 H, s), 8.98 (1 H, d, J 2.4), 9.57 (1 H, s); m/z (ES⁺) 412 (M+H⁺).

### EXAMPLE 81

### 2-[(cis-2,6-Dimethylmorpholin-4-yl)methyl]-5-methyl-6-quinolin-7-yl-N-[5-trifluoromethylpyridin-2-yl]pyrimidin-4-amine

To a suspension of Example 80 (500 mg, 1.22 mmol) in anhydrous dichloromethane (15 ml) was added pyridine (0.108 ml, 1.34 mmol) followed by methane sulfonyl chloride (0.094 ml, 1.22 mmol), and the resulting mixture stirred at room temperature overnight. Further pyridine (0.108 ml, 1.34 mmol), and methane sulfonyl chloride (0.094 ml, 1.22 mmol) was added, and mixture heated at reflux overnight. DMF (10 ml) was added to the mixture followed by cis-2,6-dimethylmorpholine (0.301 ml, 2.44 mmol) and the mixture heated at 90° C. overnight. The cooled mixture was partitioned between EtOAc and water, extracted with EtOAc (×3), and the combined EtOAc layers washed with water, sat. NaCl, dried over Na₂SO₄, filtered and evaporated. The residue was purified by column chromatography on silica (elutent 3% [2M NH₃ in MeOH] in DCM), and the product triturated with diethyl ether/isohexanes, filtered and dried to give the title compound (35 mg, 5%). ¹H NMR (500 MHz, CDCl₃) 1.18 (6 H, d, J 6.3), 2.04 (2 H, t, J 10.8), 2.39 (3 H, s), 3.01 (2 H, d, J 10.8), 3.82 (4 H, m), 7.47 (1 H, dd, J 8.4 and 4.3), 7.69 (1 H, s), 7.76 (1 H, dd, J 8.4 and 1.5), 7.95 (2 H, d, J 8.5), 8.22 (2 H, m), 8.56 (1 H, s), 8.90 (1 H, d), 8.99 (1 H, d, J 4.3 and 1.5); m/z (ES⁺) 509 (M+H⁺).

### EXAMPLE 82

### 5-Methyl-6-(1,8-naphthyridin-2-yl)-N-[4-trifluoromethylphenyl]pyrimidin-4-amine

To a mixture of Description 95 (200 mg, 0.7 mmol), Description 92 (115 mg, 0.7 mmol) and Pd(PPh₃)₄ (80 mg, 0.07 mmol) in anhydrous 1,4-dioxane (4 ml) was added hexamethylditin (0.145 ml, 0.7 mmol). The mixture was heated at 190° C. for 15 min in a microwave reactor (Personal Chemistry—Smith synthesizer). The cooled reaction mixture was loaded directly onto a silica gel chromatography column and eluted with 2% MeOH+0.5% NH₄OH in DCM. The product was further purified by mass directed HPLC to give the title compound as a white solid (50 mg, 18%). ¹H NMR (500 MHz, CDCl₃) 2.70 (3 H, s), 6.94 (1 H, s), 7.58 (1 H, dd, J 8.1 and 4.2), 7.64 (2 H, d, J 8.6), 7.84 (2 H, d, J 8.6), 8.25 (1 H, d, J 8.5), 8.29 (1 H, dd, J 8.1 and 1.9), 8.38 (1 H, d, J 8.4), 8.81 (1 H, s), 9.20 (1 H, dd, J 4.2 and 1.9); m/z (ES⁺) 382 (M+H⁺).

Examples 83-89 were made from the indicated compounds according to the procedure of Example 82.

### EXAMPLE 83

### 5-Methyl-6-(1,8-naphthyridin-2-yl)-N-[5-trifluoromethylpyridin-2-yl]pyrimidin-4-amine

Description 88 and Description 92 gave a white solid (50 mg, 18%). ¹H NMR (500 MHz, CDCl₃) 2.75 (3 H, s), 7.58 (1 H, dd, J 8.1 and 4.2), 7.79 (1 H, s), 7.97 (1 H, dd, J 8.9 and 1.9), 8.24 (1 H, d, J 8.4), 8.29 (1 H, dd, J 8.1 and 1.8), 8.39 (1 H, d, J 8.4), 8.57 (1 H, s), 8.80 (1 H, d, J 8.9), 8.89 (1 H, s), 9.21 (1 H, dd, J 4.2 and 1.9); m/z (ES⁺) 383 (M+H⁺).

### EXAMPLE 84

### 5-Isopropyl-6-(1,8-naphthyridin-2-yl)-N-[4-trifluoromethylphenyl]pyrimidin-4-amine

Description 92 and Description 96 gave a white solid (70 mg, 27%). ¹H NMR (500 MHz, CDCl₃) 1.50 (6 H, d, J 7.4), 3.86 (1 H, quintet, J 7.4), 7.00 (1 H, s), 7.57 (1 H, dd, J 8.1 and 4.2), 7.64 (2 H, d, J 8.5), 7.82 (2 H, d, J 8.5), 8.00 (1 H, d, J 8.4), 8.28 (1 H, dd, J 8.1 and 1.9), 8.37 (1 H, d, J 8.4), 8.75 (1 H, s), 9.21 (1 H, dd, J 4.2 and 1.9); m/z (ES⁺) 410 (M+H⁺).

### EXAMPLE 85

### 5-tert-Butyl-6-(1,8-naphthyridin-2-yl)-N-[4-trifluoromethylphenyl]pyrimidin-4-amine

Description 92 and Description 97 gave a white solid (70 mg, 13%). ¹H NMR (500 MHz, CDCl₃) 1.41 (9 H, s), 7.01 (1 H, s), 7.55 (1 H, dd, J 8.1 and 4.3), 7.64(2 H, d, J 8.5), 7.76 (2H, d, J 8.5), 7.89 (1 H, d, J 8.3), 8.26 (1 H, dd, J 6.5 and 1.7), 8.34 (1 H, d, J 8.3), 8.60 (1 H, s), 9.17 (1 H, dd, J 4.3 and 1.7); m/z (ES⁺) 424 (M+H⁺).

### EXAMPLE 86

### 6-(5-Fluoroquinolin-7-yl)-5-methyl-N-[5-trifluoromethylpyridin-2-yl]pyrimidin-4-amine

Description 88 and Description 90 gave a white solid (27mg, 15%). ¹H NMR (400 MHz, DMSO) 2.39 (3 H, s), 7.68 (1 H, dd, J 10.6 and 1.3), 7.73 (1 H, dd, J 8.5 and 4.2), 8.06 (1 H, s), 8.18 (1 H, dd, J 9.0 and 2.3), 8.36 (1 H, d, J 8.9), 8.58 (1 H, d, J 6.8), 8.73 (1 H, s), 8.80 (1 H, s), 9.08 (1 H, dd, J 4.2 and 1.6), 9.65(1 H, s).

### EXAMPLE 87

### 5-Methyl-6-(1,5-naphthyridin-3-yl)-N-[4-trifluoromethylphenyl]pyrimidin-4-amine

3-Bromo-1,5-naphthyridine [Journal of Organic Chemistry (1968) 33(4), 1384-7] and Description 95 gave a white solid (30 mg, 36%). ¹H NMR (400 MHz, DMSO-d₆) 2.38 (3 H, s), 7.72 (2 H, d, J 8.7), 7.88 (1 H, dd, J 8.4 and 4.1), 8.03 (2 H, d, J 8.5), 8.53 (1 H, d, J 8.6), 8.62 (1 H, d, J 2.0), 8.70 (1 H, s), 9.03 (1 H, s), 9.10 (1 H, dd, J 4.1 and 1.6), 9.21 (1 H, d, J 2.2); m/z (ES⁺) 382 (M+H⁺).

### EXAMPLE 88

### 5-Methyl-6-(1,5-naphthyridin-3-yl)-N-[5-trifluoromethylpyridin-2-yl]pyrimidin-4-amine

3-Bromo-1,5-naphthyridine [Journal of Organic Chemistry (1968) 33(4), 1384-7] and Description 88 gave a white solid (4 mg). ¹H NMR (500 MHz, DMSO-d₆) 2.41 (3 H, s), 7.89 (1 H, dd, J 8.4 and 4.1), 8.20 (1 H, dd, J 9.0 and 2.5), 8.37 (1 H, d, J 8.9), 8.53 (1 H, d, J 8.3), 8.62 (1 H, d, J 1.5), 8.74 (1 H, s), 8.83 (1 H, s), 9.11 (1 H, dd, J 4.1 and 1.3), 9.22 (1 H, d, J 2.0), 9.70 (1 H, s). m/z (ES⁺) 383 (M+H⁺).

### EXAMPLE 89

### 5-Methyl-6-(1-methyl-1H-benzimidazol-5-yl)-N-[4-trifluoromethylphenyl]pyrimidin-4-amine

Description 95 and 5-bromo-1-methylbenzimidazole [J. Chem. Soc., Perkin Trans 2 (1978), 9, 865] gave a white solid (40 mg, 26%). ¹H NMR (400 MHz, DMSO-d₆) 8.85 (1 H, s), 8.61 (1 H, s), 8.27 (1 H, s), 8.01 (2 H, d, J 8.4), 7.83 (1 H, s), 7.77-7.63 (3 H, m), 7.50 (1 H, d, J 8.4), 3.90 (3 H, s), 2.31 (3 H, s); m/z (ES⁺) 384 (M+H⁺).

### EXAMPLE 90

### 6-(1 H-Benzimidazol-6-yl)-N-[4-trifluoromethylphenyl]pyrimidin-4-amine

A mixture of Description 99 (210 mg, 0.43 mmol) and tetrabutylammonium fluoride (1.0M soln in THF: 0.86 ml, 0.86 mmol) in THF was heated at 60° C. overnight. The cooled mixture was diluted with EtOAc (50 ml), and washed with water then brine, dried over Na₂SO₄, filtered, and evaporated. The residue was purified by PREP-TLC (eluent: 7.5% MeOH in DCM+0.5% NH₄OH) to give the title compound as a white solid. (110 mg, 72%). ¹H NMR (400 MHz, DMSO-d₆) 7.38 (1 H, d, J 0.9), 7.70 (3 H, m), 7.92 (1 H, br d, J 8.2), 7.99 (2 H, d, J 8.6), 8.32 (1 H, br s), 8.33 (1 H, s), 8.80 (1 H, s), 10.04 (1 H, s), 12.69 (1 H, br s); m/z (ES⁺) 356 (M+H⁺).

### EXAMPLE 91

### 5-Bromo-6-quinolin-7-yl-N-[4-trifluoromethylphenyl]pyrimidin-4-amine

A mixture of Example 7 (370 mg, 1 mmol) and N-bromosuccinimide (180 mg, 1 mmol) in chloroform (5 ml) was heated at reflux for 30 min. More N-bromosuccinimide (100 mg, 0.56 mmol) was added and heating continued for 1 hour. The mixture was cooled, diluted with dichloromethane (15 ml), then washed with water, dried over Na₂SO₄, filtered and evaporated. A 200 mg portion was purified by PREP-TLC (eluent: 5% MeOH in DCM+0.5% NH₄OH) to give the title compound as a white solid (120 mg, 27%). ¹H NMR (400 MHz, CDCl₃) 7.49 (1 H, dd, J 8.6 and 4.3), 7.67 (2 H, d, J 8.6), 7.73 (1 H, s), 7.86 (3 H, d, J 8.6), 7.96 (1 H, d, J 8.2), 8.23 (1 H, d, J 8.6), 8.51 (1 H, s), 8.76 (1 H, s), 9.00 (1 H, dd, J 4.3 and 1.6); m/z (ES⁺) 445/447 (M+H⁺).

### EXAMPLE 92

### 5-Methyl-2-methylthio-6-quinolin-7-yl-N-[4-trifluoromethylphenyl]pyrimidin-4-amine

A suspension of Description 104 (500 mg, 1.7 mmol), 4-aminobenzotrifluoride (207 μl, 1.7 mmol) and ethanol (4 ml) was heated at 160° C. for 80 min in a microwave (Personal Chemistry—Smith synthesizer). The cooled reaction mixture was filtered, and washed with a small amount of ethanol to give a white solid (300 mg, 42%). ¹H NMR (500 MHz, DMSO-d₆) 2.28 (3 H, s), 2.50 (3 H, s), 7.62 (1 H, dd, J 8.3 and 4.1), 7.72 (2 H, d, J 8.5), 7.80 (1 H, d, J 8.4), 7.99 (2 H, d, J 8.4), 8.11 (1 H, d, J 8.3), 8.16 (1 H, s), 8.46 (1 H, d, J 8.1), 8.99 (2 H, m); m/z (ES⁺) 427(M+H⁺).

### EXAMPLE 93

### 5-Methyl-4-quinolin-7-yl-6-{4-trifluoromethylphenylamino} pyrimidine-2-carbonitrile

To a solution of Example 124 (100 mg, 0.22 mmol) in DMSO (2 ml) was added sodium cyanide (13 mg, 0.27 mmol). The mixture was stirred at room temperature for 3 days, and then partitioned between water and ethyl acetate. The aqueous phase was extracted with ethyl acetate and the combined organic extracts were washed with brine, dried over Na₂SO₄, filtered and evaporated. The residue was purified by column chromatography on silica (eluant: 1:1 ethyl acetate: hexane then ethyl acetate). Further purification by mass directed HPLC gave a solid (34 mg, 38%). ¹H NMR (360 MHz, DMSO-d₆) 2.40 (3 H, s), 7.65 (1 H, dd, J 8.3 and 4.2), 7.78-7.84 (3 H, m), 7.93 (2 H, d, J 8.5), 8.15 (1 H, d, J 8.5), 8.22 (1 H, s), 8.49 (1 H, d, J 8.3), 9.01 (1 H, dd, J 4.2 and 1.6), 9.43 (1 H, s); m/z (ES⁺) 406 (M+H⁺).

### EXAMPLE 94

### 6-(3-Fluoroquinolin-7-yl)-5-methyl-N-[5-trifluoromethylpyridin-2-yl]pyrimidin-4-amine

To Description 111 (200 mg, 0.73 mmol) and Description 88 (106 mg, 0.37 mmol) in 1,4-dioxane (4 ml) was added 2M Na₂CO₃ (370 μl, 0.73 mmol) and Pd(dppf)₂C₂ (8 mg, 0.01 mmol). The mixture was heated at 170° C. for 20 min in a microwave (Personal Chemistry—Smith synthesizer). The cooled reaction mixture was loaded directly onto a silica gel chromatography column and eluted using 2% MeOH in DCM. The product was further purified by mass directed HPLC to give the title compound as a white solid (6 mg, 4%). ¹H NMR (400 MHz, DMSO-d₆) 2.37 (3 H, s), 7.88 (1 H, d, J 8.5), 8.16 (2 H, m), 8.23 (1 H, s), 8.36 (2 H, d, J 9.2), 8.72 (1 H, s), 8.80 (1 H, s), 9.03 (1 H, d, J 2.7), 9.62 (1 H, s); m/z (ES⁺) 400 (M+H⁺).

### EXAMPLE 95

### 5-Methyl-2-(2-methylpyrrolidin-1-yl)-6-quinolin-7-yl-N-[5-trifluoromethylpyridin-2-yl]pyrimidin-4-amine

To a suspension of Example 125 (200 mg, 0.44 mmol) in 1,4-dioxane (4 ml) was added 2-methylpyrrolidine (220 μl, 2.2 mmol). The mixture was heated at 180° C. for 20 min in a microwave (Personal Chemistry—Smith synthesizer). The cooled reaction mixture was loaded directly onto a silica gel chromatography column and eluted using 1:1 ethyl acetate-hexane to give a white solid (140 mg, 70%). ¹H NMR (500 MHz, DMSO-d₆) 1.25 (3 H, d, J 6.3), 1.68 (1 H, bs), 1.92 (1 H, bs), 2.05 (2 H, bs), 2.20 (3 H, s), 3.51 (1 H, bs), 3.61 (1 H, bs), 4.24 (1 H, bs), 7.60 (1 H, dd, J 8.3 and 4.2), 7.77 (1 H, dd, J 8.4 and 1.5), 8.08 (1 H, d, J 8.4), 8.11 (1 H, s), 8.16 (1 H, dd, J 8.9 and 2.2), 8.44 (1 H, d, J 7.6), 8.55 (1 H, d, J 8.8), 8.67 (1 H, s), 8.96 (1 H, dd, J 4.2 and 1.6), 9.10 (1 H, s); m/z (ES⁺) 465 (M+H⁺).

### EXAMPLE 96

### 5-Methyl-2-morpholin-4-yl-6-quinolin-7-yl-N-[5-trifluoromethylpyridin-2-yl]pyrimidin-4-amine

Prepared from Example 125 and morpholine according to the procedure of Example 95 to give a white solid (150 mg, 74%). ¹H NMR (500 MHz, DMSO-d₆) 2.22 (3 H, s), 3.69 (8 H, s), 7.60 (1 H, dd, J 8.3 and 4.2), 7.78 (1 H, dd, 8.3 and 1.5), 8.09 (1 H, d, J 8.4), 8.13 (1H, s), 8.16 (1 H, dd, J 9.1 and 2.4), 8.28 (1 H, d, J 8.8), 8.44 (1 H, d, J 8.2), 8.68 (1 H, s), 8.97 (1 H, dd, J 4.2 and 1.6), 9.31 (1 H, s); m/z (ES⁺) 467 (M+H⁺).

### EXAMPLE 97

### 5-Methyl-6-quinolin-7-yl-2-(2,2,2-trifluoroethoxy)-N-[5-trifluoromethylpyridin-2-yl]pyrimidin-4-amine

To a solution of 2,2,2-trifluoroethanol (64 μl, 0.88 mmol) in anhydrous THF (5 ml) was added sodium hydride (60% dispersion in mineral oil) (35 mg, 0.88 mmol). The resulting mixture was stirred for 10 min, and then added to a solution of Example 125 (200 mg, 0.44 mmol) in anhydrous DMF (15 ml) and this mixture was heated at 120° C. for 6 hours. Water was added to the cooled reaction mixture, and this was extracted twice with ethyl acetate. The combined organic extracts were washed with water then brine, dried over MgSO₄, filtered and evaporated. The residue was purified by column chromatography on silica (eluant: 2:1 ethyl acetate: hexane) to give a white solid (45 mg, 22%). ¹H NMR (400 MHz, DMSO-d₆) 2.33 (3 H, s), 5.04 (2 H, q, J 9.0), 7.63 (1 H, dd, J 8.3 and 4.2), 7.84 (1 H, dd, J 8.4 and 1.7), 8.13 (1 H, d, J 8.4), 8.23 (2 H, m), 8.33 (1 H, d, J 8.8), 8.47 (1 H, d, J 7.4), 8.75 (1 H, s), 8.99 (1 H, dd, J 4.2 and 1.7), 9.72 (1 H, s); m/z (ES⁺) 480 (M+H⁺).

### EXAMPLES 98 and 99

### 7-(5-Methyl-6-{5-trifluoromethylpyridin-2-ylaminol}pyrimidin-4-yl)quinolin-4-ol (Example 98) and 5-5-methyl-6-{5-trifluoromethylpyridin-2-ylamino}pyrimidin-4-ylquinolin-4-ol (Example 99)

Prepared from Description 113 according to the procedure of Description 35 to give the two title compounds which were separated by preparative HPLC to give 2 white solids, Example 98 (73 mg, 8%). ¹H NMR (400 MHz, DMSO-d₆) 2.33 (3 H, s), 6.10 (1 H, d, J 7.4), 7.50 (1 H, d, J 8.4), 7.75 (1 H, s), 7.98 (1 H, d, J 7.4), 8.18 (2 H, m), 8.35 (1 H, d, J 8.8), 8.72 (1 H, s), 8.77 (1 H, s) and Example 99 (56 mg, 6%). ¹H NMR (400 MHz, DMSO-d₆) 1.91 (3 H, s), 5.90 (1 H, dd, J 7.4 and 1.1), 7.00 (1 H, dd, J 7.0 and 1.2), 7.63 (1 H, dd, J 8.4 and 1.2), 7.70 (1 H, dd, J 8.4 and 7.1), 7.89 (1 H, dd, J 7.3 and 6.0), 8.15 (1 H, dd, J 9.0 and 2.5), 8.37 (1 H, d, J 9.0), 8.61 (1 H, s), 8.67 (1 H, s), 9.34 (1 H, s), 11.84 (1 H, d, J 5.3).

### EXAMPLE 100

### 2-Methyl-6-quinolin-7-yl-N⁴-[4-trifluoromethylphenyl]pyrimidine-4,5-diamine

Prepared from Description 125 and 4-aminobenzotrifluoride in 1,4-dioxane according to the procedure of Example 92 to give a white solid (40 mg, 42%). ¹H NMR (360 MHz, DMSO-d₆) 2.46 (3 H, s), 5.05 (2 H, s), 7.59 (1 H, dd, J 8.3 and 4.2), 7.68 (2 H, d, J 8.7), 7.90 (1 H, dd, J 8.4 and 1.6), 8.05 (2 H, d, J 8.6), 8.10 (1 H, d, J 8.5), 8.32 (1 H, s), 8.43 (1 H, d, J 8.2), 8.85 (1 H, s), 8.96 (1 H, dd, J 4.2 and 1.7); m/z (ES⁺) 396 (M+H⁺).

### EXAMPLE 101

### 4-Quinolin-7-yl-6-{4-trifluoromethylphenylamino}pyrimidin-2-ol

A suspension of Example 14 (2.4 g, 6.1 mmol) in 2N HCl (100 ml) was heated at reflux for 48 hours. The reaction mixture was cooled to room temperature and neutralised with sat. NaHCO₃. The solid formed was filtered and dried overnight in a vacuum oven to give an off white solid (1.8 g, 80%). ¹H NMR (360 MHz, DMSO-d₆) 6.46 (1 H, s), 7.67 (1 H, dd, J 8.3 and 4.2), 7.72 (2H, d, J 8.5), 7.92 (1 H, dd, J 8.5 and 1.8), 8.08 (2 H, d, J 8.0), 8.18 (1 H, d, J 8.6), 8.46 (1 H, s), 8.50 (1H, d, J 7.6), 9.03 (1 H, dd, J 4.2 and 1.7); m/z (ES⁺) 383 (M+H⁺).

### EXAMPLE 102

### 2-Chloro-6-quinolin-7-yl-N-[4-trifluoromethylphenyl]pyrimidin-4-amine

Prepared from Example 101 according to the procedure of Description 36 to give a light brown solid. ¹H NMR (500 MHz, DMSO-d₆) 7.54 (1 H, s), 7.62 (1 H, dd, J 8.2 and 4.1), 7.73 (2 H, d, J 8.5), 7.90 (2 H, d, J 8.3), 8.14 (1 H, d, J 8.6), 8.20 (1 H, dd, J 8.5 and 1.6), 8.45 (1 H, d, J 8.1), 8.64(1 H, s), 9.00 (1 H, dd, J 4.1 and 1.6); m/z (ES⁺) 401 (M+H⁺).

### EXAMPLE 103

### 2-Morpholin-4-yl-6-quinolin-7-yl-N-[4-trifluoromethylphenyl]pyrimidin-4-amine

A suspension of Example 102 (50 mg, 0.12 mmol), morpholine (55 μl, 0.6mmol) and 1,4-dioxane (2 ml) were heated at 160° C. for 20 mins in a microwave (Personal Chemistry—Smith synthesizer). The cooled reaction was loaded directly onto a preparative TLC plate and eluted using 4% MeOH in DCM to give a white solid (10 mg, 18%). ¹H NMR (500 MHz, DMSO-d₆) 3.75 (4 H, bs), 3.84 (4 H, bs), 6.86 (1 H, s), 7.59 (1 H, dd, J 8.2 and 4.2), 7.69(2 H, d, J 8.6), 7.93 (2 H, d, J 8.4), 8.11 (1 H, d, J 8.5), 8.24 (1 H, d, J 8.5), 8.43 (1 H, d, J 8.0), 8.63 (1 H, s), 8.98 (1 H, d, J 2.5), 9.89 (1 H, s); m/z (ES⁺) 452 (M+H⁺).

### EXAMPLE 104

### 2-(4-Phenylpiperazin-1-yl)-6-quinolin-7-yl-N-[4-trifluoromethylphenyl]pyrimidin-4-amine

Prepared from Example 102 and 1-phenylpiperazine according to the procedure of Example 103 to give a white solid. ¹H NMR (500 MHz, DMSO-d₆) 4.03 (4 H, bs), 6.82 (1 H, t, J 7.2), 6.85 (1 H, s), 7.03 (2 H, d, J 8.0), 7.26 (2 H, t, J 7.9), 7.60 (1 H, dd, J 8.2 and 4.2), 7.71 (2 H, d, J 8.4), 7.96 (2 H, d, J 8.5), 8.13 (1 H, d, J 8.4), 8.25 (1 H, dd, J 8.3 and 1.7), 8.44 (1 H, d, J 7.9), 8.65 (1 H, s), 8.98 (1 H, dd, J 4.2 and 1.7), 9.88 (1 H, s); nd/z (ES⁺) 527 (M+H⁺).

### EXAMPLE 105

### 6-quinolin-7-yl-N²-(2,2,2-trifluoroethyl)-N-⁴-[4-trifluoromethylphenyl]pyrimidine-2,4-diamine

Prepared from Example 102 and 2,2,2-trifluoroethylamine according to the procedure of Example 103 to give a white solid. ¹H NMR (500 MHz, DMSO-d₆) 6.93 (1 H, s), 7.61 (1 H, dd, J 8.2 and 4.1), 7.66 (2 H, d, J 8.6), 7.72 (1 H, bs), 8.04 (2 H, bs), 8.13 (1 H, d, J 8.5), 8.22 (1 H, d, J 8.2), 8.44 (1 H, d, J 7.2), 8.65 (1 H, s), 8.99 (1 H, dd, J 4.1 and 1.6), 10.00 (1 H, s); m/z (ES⁺) 464 (M+H⁺).

### EXAMPLE 106

### 4-Methyl-6-quinolin-7-yl-N-[4-trifluoromethylphenyl]-1,3,5-triazin-2-amine

Prepared from Description 126 and Description 11 according to the procedure of Description 1 to give a white solid (80 mg, 18%). ¹H NMR (400 MHz, CDCl₃) 2.67 (3 H, s), 7.45 (1 H, br s), 7.48 (1 H, dd, J 8.3 and 4.2), 7.69 (2 H, d, J 8.6), 7.89-7.95 (3 H, m), 8.22 (1 H, d, J 7.9), 8.60 (1 H, dd, J 8.6 and 1.6), 9.03 (1 H, s), 9.26-9.27 (1 H, m); m/z (ES⁺) 382 (M+H⁺).

### EXAMPLE 107

### 2-(1,1-Dimethylethyl)-5-methyl-6-quinolin-7-yl-N-[4-trifluoromethylphenyl]pyrimidin-4-amine

Description 127 (0.12 g, 0.38 mmol) and 4-trifluoromethylaniline (0.12 g, 0.74 mmol) in dioxane (4 ml) was treated with 2N HCl in ether (0.5 ml) and the resulting solution heated at 170° C. for 20 mins in a microwave apparatus. The precipitate was collected by filtration and the desired product isolated by ion exchange chromatography as a white solid (45 mg, 27%). ¹H NMR (360 MHz, CDCl₃) 1.46 (9 H, s), 2.37 (3 H, s), 6.73 (1 H, s), 7.47 (1 H, dd, J 8.4 and 4.2), 7.64 (2 H, d, J 8.6), 7.86 (1 H, dd, J 8.4 and 1.6), 7.95 (2 H, d, J 8.4), 7.95 (1 H, d, J 8.4), 8.23 (1 H, d, J 8.4), 8.34 (1 H, s), 9.0 (1 H, d, J 1.6); m/z (ES⁺) 436 (M+H⁺).

### EXAMPLE 108

### 5-Methyl-6-quinolin-5-yl-N-[5-trifluoromethylpyridin-2-yl]pyrimidin-4-amine

Prepared from Example 99 according to the procedures of Descriptions 36 and 26 respectively. ¹H NMR (360 MHz, DMSO-d₆) 2.05 (3 H, s). 7.52 (1H, dd, J 4.1, 8.5) 7.61 (1H, dd, J 7.1 and 1.1), 7.88 (1H, dd, J 8.5 and 7.1) 7.97 (1H, d, J 8.5) 8.22-8.14 (2H, m), 8.43 (1H, d, J 8.8), 8.73 (1H, br. s), 8.80 (1H, s), 8.96 (1H, dd, J 4.1 and 1.6), 9.59 (1H, s); m/z (ES⁺) 382 (M+H⁺).

### EXAMPLE 109

### 2-(Morpholin-4-ylmethyl)-6-quinolin-7-yl-N-[4-trifluoromethylphenyl]pyrimidin-4-amine

A mixture of Description 130 (122 mg, 0.327 mmol), Description 11 (167 mg, 0.654 mmol), Pd(dppf)Cl₂ (12 mg, 0.016 mmol) and 2M sodium carbonate (654 □l, 1.31 mmol) in dioxane (3 ml) was degassed and heated at 140° C. for 30 mins. The cooled mixture was diluted with ethyl acetate, washed with water and brine, dried over sodium sulfate, filtered and concentrated. The residue was purified by flash chromatography on silica gel eluting with 5% MeOH in 1:1 isohexane-ethyl acetate increasing polarity to 10% MeOH in 1:1 isohexane-ethyl acetate. The product was then recrystallised from toluene to give a beige coloured solid (40 mg, 26%). ¹H NMR (500 MHz, DMSO-d₆) 2.66 (4 H, m), 3.66 (4 H, m), 3.76 (2 H, s), 7.41 (1 H, s), 7.61 (1 H, dd, J 8.2 and 4.1), 7.71 (2 H, d, J 8.6), 8.06 (2 H, d, J 8.5), 8.15 (1 H, d, J 8.6), 8.26 (1 H, dd, J 8.5 and 1.4), 8.45 (1 H, d, J 8.0), 8.69 (1 H, s), 9.00 (1 H, dd, J 4.1 and 1.5), 10.15 (1 H, s); m/z (ES⁺) 466 (M+H⁺).

### EXAMPLE 110

### (4-Quinolin-7-yl-6-{4-trifluoromethylphenylamino}pyrimidin-2-yl)methanol

Prepared from Description 132 and Description 11 according to the procedure of Example 109 to give a beige coloured solid. ¹H NMR (500 MHz, DMSO-d₆) 4.63 (2 H, d, J 6.2), 5.22 (1 H, t, J 6.3), 7.42 (1 H, s), 7.62 (1 H, dd, J 8.2 and 4.1), 7.70 (2 H, d, J 8.6), 8.07 (2 H, d, J 8.5), 8.15 (1 H, d, J 8.6), 8.32 (1 H, dd, J 8.5 and 1.5), 8.45 (1 H, d, J 8.0), 8.74 (1 H, s), 9.00 (1 H, dd, J 4.1 and 1.6), 10.14 (1 H, s); m/z (ES⁺) 397 (M+H⁺).

### EXAMPLE 111

### 2-(1H-Imidazol-1-ylmethyl)-6-quinolin-7-yl-N-4-trifluoromethylphenyl]pyrimidin-4-amine

A solution of methanesulphonic anhydride (28.6 mg, 0.164 mmol) in DCM (1 ml) was added to an ice-cooled suspension of Example 110 (50 mg, 0.126 mmol) and triethylamine (61 □l, 0.442 mmol) in DCM (2 ml). The mixture was warmed to room temperature and stirred for 18 hours. More triethylamine (61 □l, 0.442 mmol)) and methanesulphonic anhydride (28.6 mg, 0.164 mmol) were added with ice-cooling, then the reaction stirred for a further 3 hours at room temperature. The mixture was diluted with ethyl acetate, washed with sodium hydrogen carbonate solution (aq) and brine, dried over sodium sulphate, filtered and concentrated to dryness to give a pale brown solid (42 mg, 70%). ¹H (360 MHz, DMSO-d₆) 3.36 (3 H, s), 5.41 (2 H, s), 7.50 (1 H, s), 7.64 (1 H, dd, J 8.3 and 4.1), 7.71 (2 H, d, J 8.7), 8.06 (2 H, d, J 8.5), 8.17 (1 H, d, J 8.6), 8.28 (1 H, d, J 8.7), 8.46 (1 H, d, J 8.4), 8.72 (1 H, s), 9.01 (1 H, d, J 4.2), 10.35 (1 H, s). To this solid (40 mg, 0.084 mmol) in ethanol (2 ml), imidazole (29 mg, 0.422 mmol) was added and the reaction stirred and heated at 80° C. for 18 hours. The cooled mixture was diluted with ethyl acetate and washed with sodium carbonate solution (aq). The organic phase was dried over sodium sulphate, filtered and concentrated. The residue was purified by flash chromatography on silica gel eluting with 20:1 DCM-MeOH to give an off-white solid (14 mg, 37%). ¹H (360 MHz, DMSO-d₆) 5.48 (2 H, s), 7.05 (1 H, s), 7.34 (1 H, s), 7.41 (1 H, s), 7.65-7.57 (3 H, m), 7.77 (2 H, d, J 8.5), 7.84 (1 H, s), 8.16 (1 H, d, J 8.6), 8.24 (1 H, d, J 8.6), 8.46 (1 H, d, J 8.0), 8.67 (1 H, s), 9.01(1 H, t, J 2.0), 10.23 (1 H, s); m/z (ES⁺) 447 (M+H⁺).

### EXAMPLE 112

### 2-Isopropyl-5-methyl-6-quinolin-7-yl-N-[4-trifluoromethyl phenyl]pyrimidin-4-amine

A mixture of Description 133 (248 mg, 0.832 mmol), 4-trifluoromethylaniline (209 □l, 1.66 mmol) and 5N HCl (5 drops) in dioxane were heated at 180° C. for 30 mins in a microwave apparatus. A precipitate was observed and the mixture was filtered. The solid was washed with ethanol and partitioned between ethyl acetate and sodium carbonate solution (aq). The organic phase was washed with brine, dried over sodium sulphate, filtered and concentrated to give a pale beige solid (230 mg, 65%). ¹H NMR (400 MHz, DMSO-d₆) 1.30 (6 H, d, J 6.9) 2.31 (3 H, s), 3.07-3.01 (1 H, m), 7.61 (1 H, dd, J 8.3 and 4.1), 7.70 (2 H, d, J 8.7), 7.81(1 H, dd, J 8.3 and 1.6), 8.11(3 H, d, J 8.4), 8.17 (1 H, s), 8.46 (1 H, d, J 7.5), 8.86 (1 H, s), 8.98 (1 H, dd, J 4.2 and 1.7); m/z (ES⁺) 423 (M+H⁺).

### EXAMPLE 113

### 2-Methylthio-6-quinolin-7-yl-N-[4-trifluoromethylphenyl]pyrimidin-4-amine

Prepared from Description 134 according to the procedure of Example 92. ¹H NMR (400 MHz, CDCl₃) 2.69 (3 H, s), 7.02 (2 H, d, J 4.1), 7.47 (1 H, dd, J 8.2 and 4.1), 7.64 (4 H, q, J 7.8), 7.92 (1 H, d, J 8.6), 8.21 (1 H, d, J 8.2), 8.29 (1 H, dd, J 8.6 and 1.8), 8.69 (1 H, s), 8.97 (1 H, dd, J 4.2 and 1.7); m/z (ES⁺) 413 (M+H⁺).

### EXAMPLE 114

### 4-Quinolin-7-yl-6-{4-trifluoromethylphenylamino}pyrimidine-2-carbonitrile

Prepared from Example 126 according to the procedure of Example 93. ¹H NMR (360 MHz, DMSO-d₆) 7.65 (1 H, dd, J 8.2 and 4.2), 7.71(1 H, s), 7.80(2 H, d, J 8.6), 7.92 (2 H, d, J 8.6), 8.18-8.26 (2 H, m), 8.48 (1 H, d, J 7.4), 8.68 (1 H, s), 9.03 (1 H, dd, J 4.2 and 1.7), 10.65 (1 H, s); m/z (ES⁺) 392 (M+H⁺).

### EXAMPLE 115

### 2-Cyclopropylmethoxy-6-quinolin-7-yl-N-[4-trifluoromethylphenyl]-pyrimidin-4-amine

Prepared from Example 126 and cyclopropylmethanol according to the procedure of Example 97. ¹H NMR (500 MHz, CDCl₃) 0.41-0.44 (2 H, m), 0.64-0.68 (2 H, m), 1.39-1.43 (1 H, m), 4.34 (2 H, d, J 7.2), 7.02 (2 H, d, J 3.8), 7.46 (1 H, dd, J 8.2 and 4.2), 7.62 (2 H, d, J 8.6), 7.66 (2 H, d, J 8.6), 7.92 (1 H, d, J 8.5), 8.20 (1 H, d, J 7.9), 8.28 (1 H, dd, J 8.5 and 1.7), 8.70(1 H, s), 8.97 (1 H, dd, J 4.1 and 1.6); m/z (ES⁺) 437 (M+H⁺).

### EXAMPLE 116

### 2-(Pyridin-3-ylmethoxy)-6-quinolin-7-yl-N-[4-trifluoromethylphenyl]-pyrimidin-4-amine

Prepared from Example 126 and pyridin-3-ylmethanol according to the procedure of Example 97. ¹H NMR (500 MHz, DMSO-d₆) 5.58 (2 H, s), 7.23 (1 H, s), 7.45 (1 H, dd, J 7.8 and 4.9), 7.62 (1 H, dd, J 8.2 and 4.1), 7.71 (2 H, d, J 8.6), 7.93-7.98 (3 H, m), 8.15 (1 H, d, J 8.6), 8.25 (1 H, dd, J 8.5 and 1.6), 8.45 (1 H, d, J 8.3), 8.56 (1 H, dd, J 4.8 and 1.6), 8.69 (1 H, s), 8.78 (1 H, d, J 1.8), 9.00 (1 H, dd, J 4.2 and 1.6), 10.23 (1 H, s); m/z (ES⁺) 474 (M+H⁺).

### EXAMPLE 117

### 2-(2-Morpholin-4-ylethoxy)-6-quinolin-7-yl-N-[4-trifluoromethylphenyl]-pyrimidin-4-amine

Prepared from Example 126 and 2-morpholin-4-ylethanol according to the procedure of Example 97. ¹H NMR (360 MHz, CDCl₃) 2.63 (4 H, m), 2.90 (2 H, t, J 5.9), 3.75 (4 H, t, J 4.6), 4.66 (2 H, t, J 5.9), 6.98 (1 H, s), 7.04 (1 H, s), 7.46 (1 H, dd, J 8.4 and 4.2), 7.60 (2 H, d, J 8.4), 7.67 (2 H, d, J 8.7), 7.92 (1 H, d, J 8.5), 8.21 (1 H, d, J 7.7), 8.27 (1 H, dd, J 8.5 and 1.7), 8.70 (1 H, s), 8.98 (1 H, m); m/z (ES⁺) 496 (M+H⁺).

### EXAMPLE 118

### 6-Quinolin-7-yl-2-(1H-tetrazol-5-yl)-N-[4-trifluoromethylphenyl]-pyrimidin-4-amine trifluoroacetic acid salt

Example 114 (36 mg, 0.092 mmol), sodium azide (60 mg, 0.92 mmol) and ammonium chloride (49 mg, 0.92 mmol) were suspended in DMF (2 ml) and heated to 120° C. for 2 hours. The mixture was poured onto water (25 ml) and filtered, washing the residue with water. The residue was dissolved in DMSO and purified using mass-directed HPLC to give the title compound (5 mg, 9%). ¹H NMR (500 MHz, DMSO-d₆) 7.69 (2 H, m), 7.78 (2 H, d, J 8.5), 8.15 (2 H, d, J 8.5), 8.25 (1 H, d, J 8.5), 8.47 (1 H, d, J 8.5), 8.54 (1 H, d, J 7.9), 8.99 (1 H, s), 9.07 (1 H, m), 10.52 (1 H, s).

### EXAMPLE 119

### 6-Quinolin-7-yl-2-trifluoromethyl-N-[4-trifluoromethylphenyl]-pyrimidin-4-amine

Description 136 (0.5 g, 1.46 mmol), Description 11 (0.41 g, 1.61 mmol), [1,1′-Bis(diphenylphosphino)ferrocene]palladium(II) chloride (53 mg, 0.073 mmol) and 2M Na₂CO₃(aq) (1.6 ml) were suspended in dioxane (5 ml) and the mixture was heated at 160° C. for 15 mins in a microwave reactor (Personal Chemistry—Emrys Optimizer®). The mixture was filtered through celite, washing the residue with EtOAc and water. The layers were separated, and the organic phase was dried (sodium sulfate) and concentrated to give a brown oily residue, which was triturated with DCM followed by diethyl ether to give a white crystalline solid (250 mg, 39%). ¹H NMR (500 MHz, CDCl₃) 7.26 (1 H, s), 7.43 (1 H, s), 7.49 (1 H, dd, J 8.6 and 4.2), 7.65 (2 H, d, J 8.6), 7.72 (2 H, d, J 8.6), 7.96 (1 H, d, J 8.6), 8.22 (1 H, d, J 7.8), 8.36 (1 H, dd, J 8.6 and 1.7), 8.70 (1 H, s), 8.99 (1 H, dd, J 4.1 and 1.6); m/z (ES⁺) 435 (M+H⁺).

### EXAMPLE 120

### 6-Quinoxalin-6-yl-N-[4-trifluoromethylphenyl]pyrimidin-4-amine

Prepared from Description 137 and 4-trifluoromethylbromobenzene according to the procedure of Example 2 to give a white solid (40 mg, 14%). ¹H NMR (400 MHz, DMSO-d₆) 7.58 (1 H, d, J 1.1), 7.73 (2 H, d, J 8.6), 8.01 (2 H, d, J 8.6), 8.28 (1 H, d, J 8.8), 8.52 (1 H, dd, J 8.8 and 2.0), 8.75 (1 H, d, J 1.9), 8.91 (1 H, d, J 0.9), 9.03 (1 H, d, J 1.8), 9.05 (1 H, d, J 1.8), 10.22 (1 H, s); m/z (ES⁺) 368 (M+H⁺).

### EXAMPLE 121

### 5-Methyl-6-quinoxalin-6-yl-N-[4-trifluoromethylphenyl]pyrimidin-4-amine

Description 138 (100 mg, 0.39 mmol) and 4-trifluoromethylaniline (126 mg, 0.78 mmol) were dissolved in dioxane (3 ml). Hydrochloric acid (1M in diethyl ether, 1 ml, 1 mmol) was added and the mixture was heated at 170° C. for 40 mins in a microwave reactor (Personal Chemistry—Emrys Optimizer®). The mixture was diluted with EtOAc, and poured onto aqueous sodium bicarbonate solution. The aqueous phase was extracted with EtOAc, the combined organic phases were washed with brine, dried over sodium sulfate and concentrated. Purification by flash chromatography using a Biotage-Horizon® HPFC system (12S cartridge, gradient elution from 20-100% ethyl acetate/isohexane) followed by mass-directed HPLC gave a cream solid (10 mg). ¹H NMR (400 MHz, DMSO-d₆) 2.36 (3 H, s), 7.75 (2 H, d, J 8.7), 8.02 (2 H, d, J 8.6), 8.08 (1 H, dd, J 8.6 and 1.9), 8.27 (1 H, d, J 8.7), 8.31 (1 H, d, J 1.9), 8.74 (1 H, s), 9.06 (2 H, m), 9.22 (1 H, s).

### EXAMPLE 122

### 5-Methyl-6-quinolin-7-yl-2-trifluoromethyl-N-[4-trifluoromethylphenyl]pyrimidin-4-amine

Prepared from Description 142 and 4-trifluoromethylbromobenzene according to the procedure of Example 2 to give a white solid (150 mg, 41%). ¹H NMR (500 MHz, DMSO-d₆) 2.41 (3 H, s), 7.64 (1 H, dd, J 8.3 and 4.2), 7.77 (2 H, d, J 8.6), 7.83 (1 H, dd, J 8.4 and 1.2), 8.02 (2 H, d, J 8.6), 8.15 (1 H, d, J 8.5), 8.23 (1 H, s), 8.48 (1 H, d, J 8.1), 9.00(1 H, dd, J 4.2 and 1.0), 9.38 (1 H, br s); m/z (ES⁺) 449 (M+H⁺).

### EXAMPLE 123

### 5-Methyl-2-methylthio-6-quinolin-7-yl-N-[5-trifluoromethylpyridin-2-yl]pyrimidin-4-amine

Prepared from Description 91 and 2-bromo-5-trifluoromethylpyridine according to the procedure of Description 7 to give a brown solid (2.24 g, 61%). ¹H NMR (400 MHz, CDCl₃) 2.37 (3 H, s), 2.63 (3 H, s), 7.45-7.53 (1 H, m), 7.62 (1 H, s), 7.80 (1 H, dd, J 8.4 and 1.6), 7.94 (1H, d, J 8.4), 7.99 (1H, dd, J 9.0 and 2.3), 8.23 (1 H, d, J 7.2), 8.26 (1H, s), 8.55 (1 H, s), 8.73 (1 H, d, J 8.8), 8.99 (1 H, dd, J 1.7 and 4.2).

### EXAMPLE 124

### 5-Methyl-2-methylsulfonyl-6-quinolin-7-yl-N-[4-trifluoromethylphenyl]pyrimidin-4-amine

To a solution of Example 92 (230 mg, 0.54 mmol) in methanol (20 ml) was added oxone® (660 mg, 1.1 mmol). The reaction mixture was stirred at room temperature overnight, then at reflux for 2 hours. The cooled reaction mixture was poured onto saturated aqueous NaHCO₃ solution and then extracted three times with ethyl acetate. The combined organic extracts were washed with brine, dried over Na₂SO₄, filtered, and evaporated to give a white solid (250 mg, 100%). ¹H NMR (400 MHz, DMSO-d₆) 2.43 (3 H, s), 3.35 (3 H, s), 7.65 (1 H, dd, J 8.2 and 4.1), 7.78 (2 H, d, J 8.4), 7.86 (1 H, d, J 8.4), 8.03 (2 H, d, J 8.5), 8.17 (1 H, d, J 8.3), 8.26 (1 H, s), 8.49 (1 H, d, J 8.2), 9.01 (1 H, s), 9.51 (1 H, s).

### EXAMPLE 125

### 5-Methyl-2-methylsulfonyl-6-quinolin-7-yl-N-[5-trifluoromethylpyridin-2-yl]pyrimidin-4-amine

Prepared from Description 122 according to the procedure of Description 123 to give a yellow solid (2.26 g, 94%). ¹H NMR (400 MHz, CDCl₃) 2.53 (3 H, s), 3.39 (3 H, s), 7.53 (1 H, dd, J 8.2 and 4.2), 7.80 (1 H, dd, J 8.4 and 1.7), 7.93 (1 H, s), 7.99 (1 H, d, J 4.2), 8.08 (1 H, dd, J 8.8 and 2.2), 8.26 (1 H, d, J 8.3), 8.29 (1 H, s), 8.57 (1 H, s), 8.85 (1 H, d, J 8.8), 9.02 (1 H, dd, J 4.2 and 1.7).

### EXAMPLE 126

### 2-Methylsulfonyl-6-quinolin-7-yl-N-[4-trifluoromethylphenyl]pyrimidin-4-amine

Prepared from Example 113 according to the procedure of Description 123. ¹H NMR (400 MHz, CDCl₃) 3.46 (3 H, m), 7.43 (1 H, s), 7.51-7.47 (1 H, m), 7.61 (2 H, d, J 8.4), 7.69 (3 H, m), 7.92 (1 H, d, J 8.6), 8.24-8.20 (2 H, m), 8.68 (1 H, s), 9.01-8.97 (1 H, m); m/z (ES⁺) 445 (M+H⁺).

1. A compound of formula I:
Y-J-L-Z (I)
wherein:
L is NR¹, O, S or CH₂;
J is a six-membered heterocycle containing one, two or three nitrogen atoms which is unsubstituted or substituted with up to three substituents, depending on the number of nitrogen atoms present, chosen independently from: halogen; hydroxy; nitro; cyano; isonitrile; C₃₋₇cycloalkyl; C₁₋₆alkyl; C₂₋₆alkenyl; C₂₋₆alkynyl; C₁₋₆alkoxy; C₃₋₇cycloalkoxy; hydroxyC₁₋₆alkyl; aminoC₁₋₆alkyl; C₁₋₆alkoxycarbonyl; haloC₁₋₆alkyl; haloC₁₋₆alkoxy; —NR²R³; —CONR²R³; —S(O)ₙC₁₋₆alkyl; —S(O)ₙNR²R³; —NHCOR¹; —NHS(O)ₙC₁₋₆alkyl; —COH, carboxy; —(CH₂)ₚNR²R³; —O(CH₂)_{q}NR²R³; —(CH₂)ₚQ; —O(CH₂)ₚQ; and phenyl, a five-membered heterocyclic ring containing one, two, three or four heteroatoms chosen from O, N and S, at most one heteroatom being O or S, or a six-membered heterocyclic ring containing one, two or three nitrogen atoms, wherein this substituent is unsubstituted or substituted by one, two or three groups chosen from halogen, hydroxy, nitro, cyano, isonitrile, C₁₋₆alkyl, C₁₋₆alkoxy, haloC₁₋₆alkyl, haloC₁₋₂alkoxy, —NR²R³, —CONR²R³, —S(O)ₙNR²R³, —NHCOR¹, NHS(O)ₙR¹, —COH, CO₂H and —S(O)ₙC₁₋₆alkyl;
when J is substituted by hydroxy, tautomerism may occur, in which case any nitrogen atom ortho or para to the resulting carbonyl group may be substituted as defined above;
Q is phenyl, a five-membered heterocyclic ring containing one, two, three or four heteroatoms chosen from O, N and S, at most one heteroatom being O or S, or a six-membered heterocyclic ring containing one, two or three nitrogen atoms, optionally substituted by halogen, C₁₋₄alkyl or haloC₁₋₄alkyl;
wherein J is substituted at positions meta to each other by L and Y;
Y is naphthalene or a fused 9- or 10-membered heteroaromatic system containing a six-membered heterocyclic ring, as defined above, or a phenyl ring, or a six-membered nitrogen-containing partially saturated ring, fused either to a six-membered heterocyclic ring as defined above or to a five-membered heterocyclic ring as defined above, Y being unsubstituted or substituted with one, two or three groups independently chosen from halogen, hydroxy, cyano, nitro, isonitrile, C₁₋₆alkyl, C₂₋₆alkenyl, C₂₋₆alkynyl, haloC₁₋₆alkyl, hydroxyC₁₋₆alkyl, aminoC₁₋₆alkyl, C₁₋₆alkoxy, C₁₋₆alkylthio, haloC₁₋₆alkoxy, —NR²R³, —CONR²R³, —S(O)ₙNR²R³, —(CH₂)ₚNR²R³, —NHCOR¹, NHS(O)ₙR¹, —COH, —CO₂H and C₁₋₆alkoxycarbonyl;
Z is phenyl, naphthyl, a six-membered heterocyclic ring containing one, two, or three nitrogen atoms or a five-membered heterocyclic ring containing one, two, three or four heteroatoms chosen from O, N and S, at most one heteroatom being O or S, Z being unsubstituted or substituted with one, two or three substituents independently chosen from halogen, hydroxy, cyano, nitro, isonitrile, C₁₋₆alkyl, C₂₋₆alkenyl, C₂₋₆alkynyl, haloC₁₋₆alkyl, hydroxyC₁₋₆alkyl, aminoC₁₋₆alkyl, C₁₋₆alkoxy, C₁₋₆alkylthio, haloC₁₋₆alkoxy, —NR²R³, —CONR²R³, —S(O)ₙNR²R³, —NHCOR¹, —NHS(O)ₙR¹, —COH, —CO₂H and C₁₋₆alkoxycarbonyl;
each R¹ is H or C₁₋₆alkyl;
each R² and R³ is chosen from H and C₁₋₆alkyl, or R² and R³, together with the nitrogen atom to which they are attached, may form a 4-6 membered ring optionally containing an oxygen atom or a further nitrogen atom, which ring is optionally substituted by C₁₋₆alkyl or Q;
each n is 0, 1 or 2;
each p is 1, 2, 3or 4;
q is 2, 3 or 4;
or a pharmaceutically acceptable salt thereof.

2. A compound according to claim 1 wherein J is an unsubstituted or substituted pyrimidine, pyrazine, pyridazine or triazine.

3. A compound according to claim 2 wherein the optional substituents on J are independently chosen from halogen, hydroxy, nitro, cyano, C₁₋₄alkyl, C₂₋₄alkenyl, C₂₋₄alkynyl, C₁₋₄alkoxy, C₁₋₄cycloalkoxy, hydroxyC₁₋₄alkyl, aminoC₁₋₄alkyl, haloC₁₋₄alkyl, haloC₁₋₄alkoxy, C₁₋₄alkoxycarbonyl, —NR²R³, C₁₋₄alkylthio, Q, CH₂Q, OCH₂Q, —(CH₂)ₚNR²R³, —CONR²R³ and —CO₂H.

4. A compound according to claim 1 wherein p is one or two.

5. A compound according to claim 1 wherein Y is an unsubstituted or substituted quinoline or isoquinoline.

6. A compound according to claim 5 wherein the substituents on Y are independently chosen from halogen, hydroxy, cyano, nitro, amino, C₁₋₄alkyl, C₂₋₄alkenyl, C₂₋₄alkynyl, haloC₁₋₄alkyl, hydroxyC₁₋₄alkyl, aminoC₁₋₄alkyl, C₁₋₄alkoxy and haloC₁₋₄alkoxy.

7. A compound according to claim 1 wherein Z is an optionally substituted pyridazinyl, phenyl or pyridyl ring.

8. A compound according to claim 7 wherein the substituents on Z are independently chosen from halogen, amino, C₁₋₄alkyl, haloC₁₋₄alkyl, hydroxyC₁₋₄alkyl, aminoC₁₋₄alkyl, C₁₋₄alkoxy and haloC₁₋₄alkoxy.

9. A compound according to claim 1 or formula Ia:
Y-J-NH-Z (Ia)
wherein:
Y is a quinoline or isoquinoline optionally substituted with one or two substituents independently chosen from hydroxy, halogen, haloC₁₋₄alkyl, C₁₋₄alkyl, C₁₋₄alkoxy, haloC₁₋₄alkoxy, nitro and amino;
J is pyridine, pyridazine, pyrazine, pyrimidine or triazine optionally substituted with one or two substituents independently chosen from hydroxy, halogen, haloC₁₋₄alkyl, C₁₋₄alkyl, C₃₋₅cycloalkyl, C₁₋₄alkoxy, hydroxyC₁₋₄alkyl, cyano, hydroxy, C₁₋₄cycloalkoxy, C₁₋₄alkylthio, haloC₁₋₄alkoxy, nitro, Q, (CH₂)ₚQ, —NR²R³, —(CH₂)ₚNR²R³ and —O(CH₂)ₚNR²R³;
wherein J is substituted at positions meta to each other by NH and Y; and
Z is phenyl or pyridyl optionally substituted with one or two substituents independently selected from halogen, haloC₁₋₄alkyl, C₁₋₄alkyl, C₁₋₄alkoxy, haloC₁₋₄alkoxy, nitro and amino;
Q is phenyl, a five-membered heterocyclic ring containing one, two, three or four heteroatoms chosen from O, N and S, at most one heteroatom being O or S, or a six-membered heterocyclic ring containing one, two or three nitrogen atoms, optionally substituted by C₁₋₄alkyl;
each R² and R³ is chosen from H and C₁₋₄alkyl, or R² and R³, together with the nitrogen atom to which they are attached, may form a six-membered ring optionally containing an oxygen atom or a further nitrogen atom, which ring is optionally substituted by C₁₋₄alkyl or Q;
p is 1, 2 or 3;
or a pharmaceutically acceptable salt thereof.

10. A compound according to claim 1 which is:
4-quinolin-8-yl-N-[4-trifluoromethylphenyl]pyrimidin-2-amine;
6-quinolin-8-yl-N-[4-trifluoromethylphenyl]pyrazin-2-amine;
5-quinolin-8-yl-N-[4-trifluoromethylphenyl]pyridazin-3-amine;
6-quinolin-8-yl-N-[4-trifluoromethylphenyl]pyrimidin-4-amine;
6-quinolin-7-yl-N-[4-trifluoromethylphenyl]pyrazin-2-amine;
4-quinolin-7-yl-N-[4-trifluoromethylphenyl]pyrimidin-2-amine;
6-quinolin-7-yl-N-[4-trifluoromethylphenyl]pyrimidin-4-amine;
5-quinolin-7-yl-N-[4-trifluoromethylphenyl]pyridazin-3-amine;
6-quinolin-7-yl-N-[5-trifluoromethylpyridin-2-yl]pyrimidin-4-amine;
6-quinolin-7-yl-N-[6-trifluoromethylpyridin-3-yl]pyrimidin-4-amine;
5-methoxy-6-quinolin-7-yl-N-[4-trifluoromethylphenyl]pyrimidin-4-amine;
5-methyl-6-quinolin-7-yl-N-[4-trifluoromethylphenyl]pyrimidin-4-amine;
5-fluoro-6-quinolin-7-yl-N-[4-trifluoromethylphenyl]pyrimidin-4-amine;
2-methoxy-6-quinolin-7-yl-N-[4-trifluoromethylphenyl]pyrimidin-4-amine;
2-methyl-6-quinolin-7-yl-N-[4-trifluoromethylphenyl]pyrimidin-4-amine;
6-(3-methylquinolin-7-yl)-N-[4-trifluoromethylphenyl]pyrimidin-4-amine;
6-quinolin-5-yl-N-[4-trifluoromethylphenyl]pyrimidin-4-amine;
6-quinolin-6-yl-N-[4-trifluoromethylphenyl]pyrimidin-4-amine;
6-(2-methylquinolin-7-yl)-N-[4-trifluoromethylphenyl]pyrimidin-4-amine;
6-(6-fluoroquinolin-7-yl)-N-[4-trifluoromethylphenyl]pyrimidin-4-amine;
6-(8-fluoroquinolin-7-yl)-N-[4-trifluoromethylphenyl]pyrimidin-4-amine;
N-[4-(trifluoromethyl)phenyl]-6-[6-trifluoromethylquinolin-7-yl]pyrimidin-4-amine;
6-(8-methylquinolin-7-yl)-N-[4-trifluoromethylphenyl]pyrimidin-4-amine;
5-fluoro-6-(8-methylquinolin-7-yl)-N-[4-trifluoromethylphenyl]pyrimidin-4-amine;
6-isoquinolin-7-yl-N-[4-trifluoromethylphenyl]pyrimidin-4-amine;
6-quinolin-8-yl-N-[4-trifluoromethylphenyl]pyridazin-4-amine;
4-quinolin-8-yl-N-[4-trifluoromethylphenyl]-1,3,5-triazin-2-amine;
5-nitro-6-quinolin-7-yl-N-[4-trifluoromethylphenyl]pyrimidin-4-amine;
6-quinolin-7-yl-N⁴-[4-trifluoromethylphenyl]pyrimidine-4,5-diamine;
5-tert-butyl-6-quinolin-7-yl-N-[4-trifluoromethylphenyl]pyrimidin-4-amine;
5-tert-butyl-6-quinolin-7-yl-N-[5-trifluoromethylpyridin-2-yl]pyrimidin-4-amine;
6-(8-ethylquinolin-7-yl)-N-[4-trifluoromethylphenyl]pyrimidin-4-amine;
6-(8-ethylquinolin-7-yl)-N-[5-trifluoromethylpyridin-2-yl]pyrimidin-4-amine;
6-(8-ethylquinolin-7-yl)-5-methyl-N-[4-trifluoromethylphenyl]pyrimidin-4-amine;
6-(8-ethylquinolin-7-yl)-5-methyl-N-[5-trifluoromethylpyridin-2-yl]pyrimidin-4-amine;
N-[2-fluoro-4-trifluoromethylphenyl]-5-methyl-6-quinolin-7-ylpyrimidin-4-amine;
6-(8-methylquinolin-7-yl)-2-trifluoromethyl-N-[4-trifluoromethylphenyl]pyrimidin-4-amine;
6-(8-methylquinolin-7-y)-2-trifluoromethyl-N-[5-trifluoromethylpyridin2-yl]pyrimidin-4-amine;
2-methoxymethyl-5-methyl-6-quinolin-7-yl-N-[5-trifluoromethylpyridin-2-yl]pyrimidin-4-amine;
5-fluoro-6-(8-fluoroquinolin-7-yl)-N-[4-trifluoromethylphenyl]pyrimidin-4-amine;
5-fluoro-6-(8-fluoroquinolin-7-yl)-N-[5-trifluoromethylpyridin-2-yl]pyrimidin-4-amine;
N-(5-methyl-6-quinolin-7-ylpyrimidin-4-yl)-6-trifluoromethylpyridazin-3-amine;
6-(1,8-naphthyridin-2-yl)-N-[4-trifluoromethylphenyl]pyrimidin-4-amine;
5-methyl-6-quinolin-8-yl-N-[4-trifluoromethylphenyl]pyrimidin-4-amine;
5-methyl-6-quinolin-8-yl-N-[5-trifluoromethylpyridin-2-yl]pyrimidin-4-amine;
N-[4-trifluoromethylphenyl]-6-[4-trifluoromethylquinolin-7-yl]pyrimidin-4-amine;
5-methyl-N-[4-trifluoromethylphenyl]-6-[4-trifluoromethylquinolin-7-yl]pyrimidin-4-amine;
5-methyl-N-[5-trifluoromethylpyridin-2-yl]-6-[4-trifluoromethylquinolin-7-yl]pyrimidin-4-amine;
6-quinolin-7-yl-N⁴-[5-trifluoromethylpyridin-2-yl]pyrimidine-4,5-diamine;
N-[3-fluoro-5-trifluoromethylpyridin-2-yl]-5-methyl-6-quinolin-7-ylpyrimidin-4-amine;
(5-methyl-4-quinolin-7-yl-6-{5-trifluoromethylpyridin-2-ylamino}pyrimidin-2-yl)methanol;
2-[(cis-2,6-dimethylmorpholin-4-yl)methyl]-5-methyl-6-quinolin-7-yl-N-[5-trifluoromethylpyridin-2-yl]pyrimidin-4-amine;
5-methyl-6-(1,8-naphthyridin-2-yl)-N-[4-trifluoromethylphenyl]pyrimidin-4-amine;
5-methyl-6-(1,8-naphthyridin-2-yl)-N-[5-trifluoromethylpyridin-2-yl]pyrimidin-4-amine;
5-isopropyl-6-(1,8-naphthyridin-2-yl)-N-[4-trifluoromethylphenyl]pyrimidin-4-amine;
5-tert-butyl-6-(1,8-naphthyridin-2-yl)-N-[4-trifluoromethylphenyl]pyrimidin-4-amine;
6-(5-fluoroquinolin-7-yl)-5-methyl-N-[5-trifluoromethylpyridin-2-yl]pyrimidin-4-amine;
5-methyl-6-(1,5-naphthyridin-3-yl)-N-[4-trifluoromethylphenyl]pyrimidin-4-amine;
5-methyl-6-(1,5-naphthyridin-3-yl)-N-[5-trifluoromethylpyridin-2-yl]pyrimidin-4-amine;
5-methyl-6-(1-methyl-1H-benzimidazol-5-yl)-N-[4-trifluoromethylphenyl]pyrimidin-4-amine;
6-(1H-benzimidazol-6-yl)-N-[4-trifluoromethylphenyl]pyrimidin-4-amine;
5-bromo-6-quinolin-7-yl-N-[4-trifluoromethylphenyl]pyrimidin-4-amine;
5-methyl-2-methylthio-6-quinolin-7-yl-N-[4-trifluoromethylphenyl]pyrimidin-4-amine;
5-methyl-4-quinolin-7-yl-6-{4-trifluoromethylphenylamino}pyrimidine-2-carbonitrile;
6-(3-fluoroquinolin-7-yl)-5-methyl-N-[5-trifluoromethylpyridin-2-yl]pyrimidin-4-amine;
5-methyl-2-(2-methylpyrrolidin-1-yl)-6-quinolin-7-yl-N-[5-trifluoromethylpyridin-2-yl]pyrimidin-4-amine;
5-methyl-2-morpholin-4-yl-6-quinolin-7-yl-N-[5-trifluoromethylpyridin-2-yl]pyrimidin-4-amine;
5-methyl-6-quinolin-7-yl-2-(2,2,2-trifluoroethoxy)-N-[5-trifluoromethylpyridin-2-yl]pyrimidin-4-amine;
7-(5-methyl-6-{5-trifluoromethylpyridin-2-ylamino}pyrimidin-4-yl)quinolin-4-ol;
5-5-methyl-6-{5-trifluoromethylpyridin-2-ylamnino}pyrimidin-4-ylquinolin-4-ol;
2-methyl-6-quinolin-7-yl-N⁴-[4-trifluoromethylphenyl]pyrimidine-4,5-diamine;
4-quinolin-7-yl-6-{4-trifluoromethylphenylamino}pyrimidin-2-ol;
2-chloro-6-quinolin-7-yl-N-[4-trifluoromethylphenyl]pyrimidin-4-amine;
2-morpholin-4-yl-6-quinolin-7-yl-N-[4-trifluoromethylphenyl]pyrimidin-4-amine;
2-(4-phenylpiperazin-1-yl)-6-quinolin-7-yl-N-[4-trifluoromethylphenyl]pyrimidin-4-amine;
6-quinolin-7-yl-N²-(2,2,2-trifluoroethyl)-N⁴-[4-trifluoromethylphenyl]pyrimidine-2,4-diamine;
4-methyl-6-quinolin-7-yl-N-[4-trifluoromethylphenyl]-1,3,5-triazin-2-amine;
2-(1,1-dimethylethyl)-5-methyl-6-quinolin-7-yl-N-[4-trifluoromethylphenyl]pyrimidin-4-amine;
5-methyl-6-quinolin-5-yl-N-[5-trifluoromethylpyridin-2-yl]pyrimidin-4-amine;
2-(morpholin-4-ylmethyl)-6-quinolin-7-yl-N-[4-trifluoromethylphenyl]pyrimidin-4-amine;
(4-quinolin-7-yl-6-{4-trifluoromethylphenylamino}pyrimidin-2-yl)methanol;
2-(1H-imidazol-1-ylmethyl)-6-quinolin-7-yl-N-[4-trifluoromethylphenyl]pyrimidin-4-amine;
2-isopropyl-5-methyl-6-quinolin-7-yl-N-[4-trifluoromethylphenyl]pyrimidin-4-amine;
2-methylthio-6-quinolin-7-yl-N-[4-trifluoromethylphenyl]pyrimidin-4-amine;
4-quinolin-7-yl-6-{4-trifluoromethylphenylamino}pyrimidine-2-carbonitrile;
2-cyclopropylmethoxy-6-quinolin-7-yl-N-[4-trifluoromethylphenyl]-pyrimidin-4-amine;
2-(pyridin-3-ylmethoxy)-6-quinolin-7-yl-N-[4-trifluoromethylphenyl]-pyrimidin-4-amine;
2-(2-morpholin-4-ylethoxy)-6-quinolin-7-yl-N-[4-trifluoromethylphenyl]-pyrimidin-4-amine;
6-quinolin-7-yl-2-(1H-tetrazol-5-yl)-N-[4-trifluoromethylphenyl]-pyrimidin-4-amine trifluoroacetic acid salt;
6-quinolin-7-yl-2-trifluoromethyl-N-[4-trifluoromethylphenyl]-pyrimidin-4-amine;
6-quinoxalin-6-yl-N-[4-trifluoromethylphenyl]pyrimidin-4-amine;
5-methyl-6-quinoxalin-6-yl-N-[4-trifluoromethylphenyl]pyrimidin-4-amine;
5-methyl-6-quinolin-7-yl-2-trifluoromethyl-N-[4-trifluoromethylphenyl]pyrimidin-4-amine;
5-methyl-2-methylthio-6-quinolin-7-yl-N-[5-trifluoromethylpyridin-2-yl]pyrimidin-4-amine;
5-methyl-2-methylsulfonyl-6-quinolin-7-yl-N-[4-trifluoromethylphenyl]pyrimidin-4-amine;
5-methyl-2-methylsulfonyl-6-quinolin-7-yl-N-[5-trifluoromethylpyridin-2-yl]pyrimidin-4-amine;
2-methylsulfonyl-6-quinolin-7-N-[4-trifluoromethylphenyl]pyrimidin-4-amine;
or a pharmaceutically acceptable salt thereof.

11. A pharmaceutical composition comprising a compound of claim 1 or a pharmaceutically acceptable salt thereof and a pharmaceutically acceptable excipient.

12. A compound of claim 1 or a pharmaceutically acceptable salt thereof for use in a method of treatment of the human or animal body by therapy.

13. A method of treating a patient suffering from a disease requiring the administration of a VR1 antagonist which comprises administering to that patient a therapeutically effective amount of a compound of claim 1 or a pharmaceutically acceptable salt thereof.

14. A method according to claim 13 wherein the disease is cough, GERD, depression or pain.
